(19) United States
(12) Patent Application Publication
(10) Pub. No.: US 2016/0025752 A1
(43) Pub. Date: Jan. 28, 2016

(54) DEVICES AND METHODS FOR DETECTING AND/OR QUANTIFYING ANALYTES IN FLUIDS

(71) Applicant: Seed Research and Development, LLC, Minneapolis, MN (US)

(72) Inventors: Frank Santiago, Minneapolis, MN (US); Leanna M. Levine, Rancho Dominguez, CA (US); Rafael de la Torre Fornell, Barcelona (ES); Magin Farre Albaladejo, Barcelona (ES)

(73) Assignee: Seed Research and Development, LLC, Minneapolis, MN (US)

(21) Appl. No.: 14/747,659

(22) Filed: Jun. 23, 2015

Related U.S. Application Data
(60) Provisional application No. 62/016,236, filed on Jun. 24, 2014.

(51) Int. Cl. G01N 33/94

(52) U.S. Cl. CPC G01N 33/948; G01N 33/946

(57) ABSTRACT

The present disclosure relates to devices and methods for determining a target analyte in a sample (e.g., a bodily fluid sample such as sweat, blood, urine, lavage, plasma, saliva, tears) using an analysis device (e.g., a bodily fluid analysis device). The device may include a lateral flow assay in some embodiments. Certain embodiments involve determining a concentration of a target analyte in a sample by generating a volumetric calculation of the sample and/or by use of a non-target analyte. The devices and methods can be used for obtaining and processing a sample to determine the presence and/or concentration of such analytes. In some cases, the methods and/or devices can be used to determine impairment (e.g., drug impairment) in a subject.

### RELATED APPLICATION

This application claims the benefit of U.S. Provisional Patent Application No. 62/016,236 filed Jun. 24, 2014, the disclosure of which is hereby incorporated by reference in its entirety for all purposes.

### FIELD

The present disclosure relates to devices for detecting and/or quantifying analytes in fluids including bodily fluids such as sweat, and methods of using such devices.

### BACKGROUND

Identification of analytes in fluids is often time-consuming and costly. For example, identification of analytes in mammals such as the human body typically requires obtaining a physiological sample, such as blood or urine, and using various laboratory methods to pretreat and analyze the sample for the target analytes. Common analytical methods include chromatography (e.g., liquid chromatography, gas chromatography, or combinations thereof), spectrometry (e.g., mass spectrometry), and immunological methods (e.g., enzyme-linked immunosorbent assay, radioimmunoassay). However, such sampling and analytical methods, while reliable, can also be invasive, time-consuming, and costly. It would therefore be beneficial to provide an analysis device that addresses these and/or other drawbacks.

### SUMMARY

In general, this disclosure is directed to devices and methods for determining the concentration of one or more analytes in a sample of fluid (e.g., bodily fluid). The subject matter disclosed herein involves, in some cases, interrelated products, alternative solutions to a particular problem, and/or a plurality of different uses of one or more systems and/or articles.

In one set of embodiments, a series of methods are provided. In one embodiment, a method of determining a concentration of a target analyte in a bodily fluid sample is provided. The method comprises introducing the bodily fluid sample comprising the target analyte into a bodily fluid analysis device, wherein the bodily fluid analysis device comprises a lateral flow assay. The method involves passing the bodily fluid sample across a test strip comprising a first band and a second band, wherein the first band is adapted and arranged to determine a quantity of the target analyte in the bodily fluid sample, and wherein the second band is adapted and arranged to determine a volume of the bodily fluid sample. The method also involves determining the concentration of the target analyte in the bodily fluid sample based on the steps of determining the quantity of the target analyte and the volume of the bodily fluid sample.

In another embodiment, a method of determining a concentration of a target analyte in a bodily fluid sample is provided. The method comprises introducing the bodily fluid sample comprising the target analyte into a bodily fluid analysis device, wherein the bodily fluid analysis device comprises a lateral flow assay. The method involves passing the bodily fluid sample across a test strip comprising a first band and a second band, wherein the first band is adapted and arranged to capture the target analyte in the bodily fluid sample, and wherein the second band is adapted and arranged to capture a non-target analyte. The method also involves determining the concentration of the target analyte in the bodily fluid sample based on the steps of capturing the target analyte and non-target analyte.

In another embodiment, a method of determining a target analyte in a bodily fluid sample is provided. The method comprises introducing the bodily fluid sample comprising the target analyte into a region of a bodily fluid analysis device, wherein the bodily fluid analysis device comprises a lateral flow assay and a sealed liquid reservoir containing a liquid, and releasing the liquid from the liquid reservoir. The method involves flowing the liquid across the region containing the bodily fluid sample comprising the target analyte, allowing the target analyte from the bodily fluid sample to bind with a first conjugate to form a target analyte-conjugate complex, and flowing the target analyte-conjugate complex across a test strip. The method also involves capturing the target analyte-conjugate complex at a first band of the test strip, and determining the target analyte.

In another set of embodiments, a series of devices are provided. In one embodiment, a bodily fluid analysis device for determining a concentration of a target analyte in a bodily fluid sample is provided. The device comprises a lateral flow assay, the lateral flow assay comprising a test strip, wherein the test strip comprises a first band and a second band, the first band adapted and arranged to determine a quantity of the target analyte in the bodily fluid sample, and the second band adapted and arranged to determine a volume of the bodily fluid sample.

In another embodiment, a bodily fluid analysis device for determining a concentration of a target analyte in a bodily fluid sample is provided. The device comprises a lateral flow assay, the lateral flow assay comprising a non-target analyte and a test strip, wherein the test strip comprises a first band and a second band, the first band adapted and arranged to determine a quantity of the target analyte in the bodily fluid sample, and the second band adapted and arranged to determine a quantity of the non-target analyte.

In another embodiment, a bodily fluid analysis device for determining a target analyte in a bodily fluid sample is provided. The device comprises a liquid reservoir containing a liquid, wherein the liquid reservoir is sealed prior to use and is adapted and arranged to release the liquid during use, and a region adapted and arranged for housing a test sample element containing a sample, wherein the region is positioned downstream of the liquid reservoir. The device also comprises a conjugate region comprising at least a first conjugate adapted and arranged to bind to the target analyte, wherein the conjugate region is positioned downstream of the liquid reservoir, and a lateral flow assay, the lateral flow assay comprising a test strip comprising a first band adapted and arranged to capture the target analyte.

In one embodiment, a method of determining a concentration of a target analyte in a fluid sample is provided. The method comprises introducing the sample comprising the target analyte into an analysis device, wherein the analysis device comprises a lateral flow assay. The method involves passing the sample across a test strip comprising a first band and a second band, wherein the first band is adapted and arranged to determine a quantity of the target analyte in the sample, and wherein the second band is adapted and arranged to determine a volume of the sample. The method also involves determining the concentration of the target analyte in the sample based on the steps of determining the quantity of the target analyte and the volume of the sample.

In another embodiment, a method of determining a concentration of a target analyte in a sample is provided. The method comprises introducing the sample comprising the target analyte into an analysis device, wherein the analysis device comprises a lateral flow assay. The method involves passing the sample across a test strip comprising a first band and a second band, wherein the first band is adapted and arranged to capture the target analyte in the sample, and wherein the second band is adapted and arranged to capture a non-target analyte. The method also involves determining the concentration of the target analyte in the sample based on the steps of capturing the target analyte and non-target analyte.

In another embodiment, a method of determining a target analyte in a sample is provided. The method comprises introducing the sample comprising the target analyte into a region of an analysis device, wherein the analysis device comprises a lateral flow assay and a sealed liquid reservoir containing a liquid, and releasing the liquid from the liquid reservoir. The method involves flowing the liquid across the region containing the sample comprising the target analyte, allowing the target analyte from the sample to bind with a first conjugate to form a target analyte-conjugate complex, and flowing the target analyte-conjugate complex across a test strip. The method also involves capturing the target analyte-conjugate complex at a first band of the test strip, and determining the target analyte.

In another set of embodiments, a series of devices are provided. In one embodiment, an analysis device for determining a concentration of a target analyte in a sample is provided. The device comprises a lateral flow assay, the lateral flow assay comprising a test strip, wherein the test strip comprises a first band and a second band, the first band adapted and arranged to determine a quantity of the target analyte in the sample, and the second band adapted and arranged to determine a volume of the sample.

In another embodiment, an analysis device for determining a concentration of a target analyte in a sample is provided. The device comprises a lateral flow assay, the lateral flow assay comprising a non-target analyte and a test strip, wherein the test strip comprises a first band and a second band, the first band adapted and arranged to determine a quantity of the target analyte in the sample, and the second band adapted and arranged to determine a quantity of the non-target analyte.

In another embodiment, an analysis device for determining a target analyte in a sample is provided. The device comprises a liquid reservoir containing a liquid, wherein the liquid reservoir is sealed prior to use and is adapted and arranged to release the liquid during use, and a region adapted and arranged for housing a test sample element containing a sample, wherein the region is positioned downstream of the liquid reservoir. The device also comprises a conjugate region comprising at least a first conjugate adapted and arranged to bind to the target analyte, wherein the conjugate region is positioned downstream of the liquid reservoir, and a lateral flow assay, the lateral flow assay comprising a test strip comprising a first band adapted and arranged to capture the target analyte.

In another aspect, a sweat analysis device for detecting an analyte in sweat is provided. The sweat analysis device comprises: a base comprising a non-permeable layer defining a top and a bottom and an opening circumferentially surrounded by at least a first, second, and third side; a top adhesive deposited on the top of the base and a bottom adhesive deposited on the bottom of the base; a sweat pad comprising an absorbent material positioned over the opening and supported by at least a portion of the first, second, and third sides of the base. The device also includes a conjugate pad comprising one or more conjugates, wherein the conjugate pad is positioned on the base in fluid communication/fluid communication with the sweat pad; a test strip comprising a test band, wherein the test strip is positioned on the base in fluid communication/fluid communication with the conjugate pad; a wicking element comprising an absorbent material, wherein the wicking element is positioned on the base in fluid communication/fluid communication with the test strip; and a liquid reservoir comprising a buffer, the liquid reservoir being capable of containing the buffer until the buffer is released by an operator, wherein the liquid reservoir is positioned on the base in fluid communication/fluid communication with the sweat pad opposite the conjugate pad, test strip, and wicking element, wherein the conjugate and the test band form an immunological assay selected for detection of the analyte in sweat.

In another aspect, a method for detecting whether an analyte is present in sweat is provided. The method comprises:

(a) obtaining a sample of sweat by placing a sweat analysis device on skin of a subject, wherein the sweat analysis device comprises a base, a sweat pad comprising an absorbent material, a conjugate pad comprising one or more conjugates, a test strip comprising a test band, a wicking element comprising an absorbent material, and a liquid reservoir comprising a buffer, and wherein the sample of sweat is absorbed into the absorbent pad;

(b) removing the sweat analysis device from the skin of the subject;

(c) releasing a liquid buffer component from the liquid reservoir;

(d) eluting the liquid buffer component toward the conjugate pad and, if the analyte is present, eluting the analyte toward the conjugate pad simultaneously with the liquid buffer component;

(e) if the analyte is present, binding the analyte to the one or more conjugates to form an analyte-conjugate complex;

(f) contacting the test band with the conjugate and, if the analyte is present, contacting the test band with the analyte-conjugate complex; and

(g) observing the test band for a detectable indication of the presence or absence of the analyte.

In another aspect, a method for detecting drug impairment of a subject is provided. The method comprises:
(a) obtaining a sample of sweat by placing a sweat analysis device on skin of a subject, wherein the sweat analysis device comprises a base, a sweat pad comprising an absorbent material, a conjugate pad comprising one or more conjugates, a test strip comprising a test band, a wicking element comprising an absorbent material, and a liquid reservoir comprising a buffer, and wherein the sample of sweat is absorbed into the absorbent pad;
(b) removing the sweat analysis device from the skin of the subject;
(c) releasing a liquid buffer component from the liquid reservoir;
(d) eluting the liquid buffer component and the analyte toward the conjugate pad;
(e) binding the analyte to the one or more conjugates to form an analyte-conjugate complex;
(f) contacting the test band with the analyte-conjugate complex;
(g) observing the test band for a detectable indication of the analyte; and
(h) observing the test band for a detectable indication of drug impairment.

In some embodiments, the analysis device described above and/or herein (and/or a device associated with a method described above and/or herein) comprises a sealed liquid reservoir containing a liquid. The liquid may be, for example, a buffered solution. In some cases, the sealed liquid reservoir comprises a puncturable seal. In some embodiments, the sealed liquid reservoir is a blister pack. In certain embodiments, the analysis device (e.g., bodily fluid analysis device) comprises a liquid releasing member adapted and arranged to release a liquid from the liquid reservoir. In some cases, the liquid releasing member comprises a puncture component.

In some embodiments, the analysis device described above and/or herein (and/or a device associated with a method described above and/or herein) comprises a region adapted and arranged for housing a test sample element which may contain a sample. In some cases, the region comprises a lid. In certain embodiments, the lid or the device body comprises a liquid releasing member. For example, the lid may comprises a compression element for applying a pressure to the test sample element upon closure of the lid. In certain embodiments, the lid comprises a locking mechanism that prevents opening of the lid after the lid has been closed.

In some embodiments, the analysis device described above and/or herein (and/or a device associated with a method described above and/or herein) comprises a test sample element. In some cases, closure of the lid releases the liquid from the liquid reservoir and into the test sample element.

In some embodiments, the analysis device described above and/or herein (and/or a device associated with a method described above and/or herein) comprises a conjugate region comprising a first conjugate adapted and arranged to bind the target analyte. In some embodiments, at least a portion of the conjugate region is positioned downstream of the liquid reservoir. In some embodiments, at least a portion of the conjugate region is positioned downstream of the region adapted and arranged for housing the test sample element. In some embodiments, at least a portion of the conjugate region is positioned above or below the region adapted and arranged for housing the test sample element.

In some embodiments, the analysis device described above and/or herein (and/or a device associated with a method described above and/or herein) includes a conjugate region comprising a second conjugate adapted and arranged to bind a non-target analyte. In some embodiments, the first and/or second conjugate comprises a detectable label. The detectable label may comprise, for example, a nanoparticle. In some cases, the nanoparticle is selected from colloidal gold nanoparticles and fluorescent nanoparticles. In some embodiments, the first and/or second conjugate comprises an antibody.

In some embodiments, the analysis device described above and/or herein (and/or a device associated with a method described above and/or herein) comprises a first band adapted and arranged to determine a quantity of the target analyte in the bodily fluid sample. In some cases, the analysis device (e.g., bodily fluid analysis device) comprises a known amount or concentration of a non-target analyte. In some embodiments, the analysis device (e.g., bodily fluid analysis device) comprises a second band adapted and arranged to determine a volume of the bodily fluid sample. In some cases, the device comprises a second band adapted and arranged to determine a quantity of the non-target analyte. In some embodiments, the test strip displays the first band and the second band, the first band comprising a complex of the target analyte and the first conjugate, the second band comprising a complex of the non-target analyte and the second conjugate. In certain embodiments, an intensity of the first band correlates with a quantity of the target analyte in the bodily fluid sample. In some embodiments, an intensity of the second band correlates with a volume of the bodily fluid sample.

In some embodiments, in the analysis device described above and/or herein (and/or a device associated with a method described above and/or herein) the liquid reservoir, absorbent material, conjugate region, and test strip are in fluid communication with one another during use. In some embodiments, the test strip comprises a material selected from nitrocellulose and hydrophilic glass fiber-based membrane treated with plastic binder. In some embodiments, the device comprises a wicking element positioned downstream of the test strip.

In some embodiments, the analysis device described above and/or herein (and/or a device associated with a method described above and/or herein) comprises a device identifying component that comprises a script for running a particular analysis; e.g., the device identifying component may comprise an RFID, a barcode, a QR code, a bluetooth-enabled component, or a Wi-Fi-enabled component. In some embodiments, the device identifying component holds information identifying a particular bodily fluid and a particular target analyte.

In some embodiments, in the analysis device described above and/or herein (and/or a device associated with a method described above and/or herein), the test strip further comprises a third band that indicates whether or not the lateral flow assay operated correctly. In some embodiments, the device comprises a results window, wherein the first band and the second band are readable through the results window.

In some embodiments, in the analysis device described above and/or herein (and/or a device associated with a method described above and/or herein), the device comprises a device body comprising a non-permeable layer defining a top and a bottom and an opening. In some cases, the device comprises a top adhesive deposited on the top of the device body and a bottom adhesive deposited on the bottom of the device body.

In some embodiments, in the analysis device described above and/or herein (and/or a device associated with a method described above and/or herein), the device comprises an absorbent material positioned over the opening and supported by at least a portion of the first, second, and third sides of the device body.

In some embodiments, in a method described above and/or herein, introducing the bodily fluid sample comprising the target analyte into the analysis device (e.g., bodily fluid analysis device) comprises placing a test sample element comprising the bodily fluid sample into a region adapted and arranged for housing the test sample element and sample. In some cases, introducing the bodily fluid sample comprising the target analyte into the analysis device (e.g., bodily fluid analysis device) comprises absorbing the bodily fluid sample with a test sample element positioned in a region adapted and arranged for housing the test sample element and sample.

In some embodiments, in a method described above and/or herein, the method comprising releasing the liquid from the liquid reservoir. In some cases, releasing the liquid from the liquid reservoir comprises puncturing a seal of the liquid reservoir. In certain embodiments, the method comprising allowing the target analyte from the bodily fluid sample to bind with a first conjugate to form an analyte-conjugate complex. The method may comprise flowing the analyte-conjugate complex across the test strip. In some cases, the method comprising capturing the analyte-conjugate complex at a first band of the test strip. In some embodiments, the method comprising passing the bodily fluid sample across a first band and a second band, wherein the first band is adapted and arranged to capture the target analyte in the bodily fluid sample, and wherein the second band is adapted and arranged to capture a non-target analyte.

In some embodiments, in a method described above and/or herein, the method comprising determining the difference between a known amount or concentration of the non-target analyte present in the analysis device (e.g., bodily fluid analysis device) prior to use, and the amount or concentration of the non-target analyte present at the second band. In some embodiments, the method comprises determining the concentration of the target analyte in the bodily fluid sample based on the steps of capturing the target analyte and non-target analyte.

In some embodiments, in a method described above and/or herein, the method comprises collecting a bodily fluid sample; introducing a liquid comprising a defined amount of a non-target analyte to the bodily fluid sample; introducing a mixture of the liquid and the bodily fluid sample to a lateral flow assay; binding the non-target analyte to a first conjugate on the lateral flow assay; binding the target analyte to a second conjugate on the lateral flow assay; measuring an intensity of the first band on a test strip on the lateral flow assay, the first band comprising a complex of the non-target analyte and the first conjugate; measuring an intensity of the second band on the test strip on the lateral flow assay, the second band comprising a complex of the target analyte and the second conjugate; and determining the concentration of the target analyte in the bodily fluid sample based on the measured intensity of the first band and the measured intensity of the second band. In some embodiments, the method comprises reading a third band on the test strip on the lateral flow assay to determine whether the lateral flow assay worked properly.

In some embodiments, in a method described above and/or herein, the method comprises determining an indication of drug impairment in a subject. In some embodiments, the indication of drug impairment is due to intake by the subject of a composition comprising one or more of benzoylecgonine, morphine, THC, methylenedioxymethamphetamine, EPO, ethyl glucuronide and/or a derivative thereof.

In some embodiments, in the analysis device described above and/or herein (and/or a device associated with a method described above and/or herein), the bodily fluid sample comprises sweat. In other embodiments, the bodily fluid sample comprises blood, saliva, or urine.

In some embodiments, in the analysis device described above and/or herein (and/or a device associated with a method described above and/or herein), the bodily fluid sample has a volume of at least 1 microliter and/or less than or equal to 500 microliters.

In some embodiments, in the analysis device described above and/or herein (and/or a device associated with a method described above and/or herein), the bodily fluid sample is sweat, wherein the sweat is collected from a person's skin with an absorbent pad, and wherein a mixture of a buffer and at least some of the sweat in the absorbent pad introduces the target analyte to the test strip.

In some embodiments, in the analysis device described above and/or herein (and/or a device associated with a method described above and/or herein), the liquid comprises a non-target analyte. In some cases, the liquid comprises a buffered solution. In some embodiments, the liquid has a volume of at least 1 microliter and/or less than or equal to 500 microliters, less than or equal to 400 microliters, less than or equal to 300 microliters, less than or equal to 200 microliters, or less than or equal to 100 microliters. In some embodiments, the buffer comprises one or more salts selected from the group consisting of NaCl, KCl, Na₂HPO₄, KH₂PO₄, CaCl₂, and MgCl₂. In some embodiments, the buffer is phosphate buffered saline.

In some embodiments, in the analysis device described above and/or herein (and/or a device associated with a method described above and/or herein), the absorbent material has a volume of at least 0.01 cm3 and/or less than or equal to 10 cm³. In some cases, the absorbent material comprises an adhesive component for adhering to a surface of skin. In some embodiments, the absorbent material comprises a sweat absorbent component, a release component, and an adhesive component, and wherein the release component facilitates separation of the absorbent material from the adhesive component. In some cases, the adhesive component comprises at least one feature that indicates when the absorbent material has been tampered with.

In some embodiments, in the analysis device described above and/or herein (and/or a device associated with a method described above and/or herein), determining the target analyte comprises determining a concentration of the target analyte. In some cases, determining the target analyte comprises determining the presence of the target analyte.

In some embodiments, in the analysis device described above and/or herein (and/or a device associated with a method described above and/or herein), the analyte is selected from the group consisting of benzoylecgonine, morphine, THC, methylenedioxymethamphetamine, EPO, and ethyl glucuronide. In some cases, the analyte provides an indication of drug impairment in the subject.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic diagram showing an analysis device according to at least one embodiment of the present disclosure.

FIG. 1B is a schematic cross-sectional side view representation of an analysis device according to at least one embodiment of the present disclosure.

FIG. 1C is a schematic cross-sectional side view representation of another bodily fluid analysis device according to at least one embodiment of the present disclosure.

FIG. 2 is a bottom view of an embodiment of a test sample element for use with an analysis device.

FIG. 3 is an expanded, schematic, side, cross-sectional view of the test sample element of

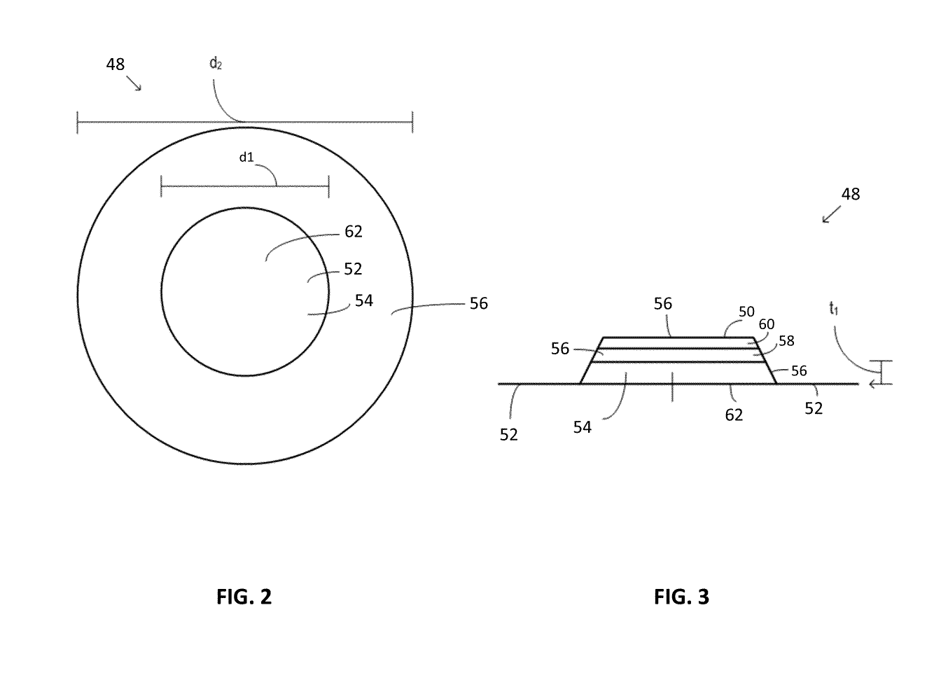

FIG. 2.

FIG. 4 is a top schematic view of an embodiment of an analysis device which may be used for analyzing a sweat sample.

FIG. 5 is a flow chart illustrating an embodiment of a method for determining the concentration of a target analyte in a bodily fluid.

FIG. 6 is a flow chart illustrating an embodiment of a method analysis for determining the concentration of a target analyte in sweat.

FIG. 7 is a top schematic view of a further embodiment of an analysis device in accordance with the present disclosure.

FIG. 8 is side schematic cross-sectional view of the analysis device of FIG. 7.

FIG. 9 is a top schematic view of another further embodiment of an analysis device in accordance with the present disclosure.

FIG. 10 is side schematic cross-sectional view of the analysis device of FIG. 9.

FIGS. 11A-11E show chronologically successive schematic top views of the analysis device of FIG. 9 in use.

FIG. 12 shows a top view of a sweat analysis device according to at least one embodiment of the present disclosure.

FIG. 13 shows an exploded view of the components of the analysis device of FIG. 12.

FIG. 14 shows a base/device body component of the analysis device of FIG. 12.

FIG. 15A shows a sweat pad component of the analysis device of FIG. 12.

FIG. 15B shows a side view of the sweat pad component of FIG. 15A.

FIG. 16 shows a conjugate pad component of the analysis device of FIG. 12.

FIG. 17 shows a test strip component of the analysis device of FIG. 12.

FIG. 18A shows a liquid reservoir component of the analysis device of FIG. 12.

FIG. 18B shows a cross sectional view of the liquid reservoir component of FIG. 18A.

FIG. 19 shows a wicking element of the analysis device of FIG. 12.

FIG. 20 shows a cover layer of the analysis device of FIG. 12.

FIG. 21 shows a bottom view of the analysis device of FIG. 12.

FIG. 22 shows the analysis device of FIG. 12 in use on a subject.

FIG. 23 is a depiction of the flow of liquids in the analysis device of FIG. 12.

### DETAILED DESCRIPTION

The present disclosure relates to devices and methods for determining a target analyte in a sample using an analysis device comprising a lateral flow assay. In some embodiments, the sample is a bodily fluid sample (e.g., sweat, blood, urine, lavage, plasma, saliva, tears). Certain embodiments involve determining a concentration of a target analyte in a sample by generating a volumetric calculation of the sample and/or by use of a non-target analyte. The methods and devices can be used for obtaining and processing a sample to determine the presence and/or concentration of such analytes. In some cases, the methods and/or devices can be used to determine impairment (e.g., drug impairment) in a subject.

In some embodiments, methods for determining the concentration of one or more target analytes present in a collected sample (e.g., bodily fluid sample) using the results of a lateral flow assay are provided. Certain methods may comprise two general aspects: 1) measuring the quantity of the analyte(s) in the sample; and 2) measuring the volume of fluid in the sample. Taking the quotient of the quantity of the analyte and the sample volume can indicate the concentration of that analyte in the sample (and in some cases, the test subject's body). Devices that can be used for such determination are also provided.

In certain embodiments, methods for determining the concentration of one or more target analytes may involve the determination of a target analyte and a non-target analyte (e.g., a volumetric analyte). For instance, the determination of the quantity of the target analyte may provide an indication of the amount of target analyte in the sample, and determination of the quantity of the non-target analyte may provide an indication of the sample volume. For purposes of this disclosure, a target analyte is an analyte being tested for in a person's bodily fluid. For purposes of this disclosure, a volumetric analyte is an example of a non-target analyte that, for example, may be added to a sample (e.g., bodily fluid sample) for purposes of measuring the sample's volume.

In some embodiments, a device and method includes a lateral flow assay comprising a test strip configured to detect a single target analyte and a volumetric analyte. Volumetric analytes will be discussed in greater detail below. In alternative embodiments, the lateral flow assay includes a test strip configured to detect multiple target analytes in the sample (e.g., bodily fluid sample) and at least one volumetric analyte. Other configurations are also possible, as described in more detail below. Additionally, although several of the examples described herein relate to non-target analytes that are used as volumetric analytes, it should be appreciated that other non-target analytes may be used in the methods and devices described herein.

As described herein, several embodiments involve determining a target analyte in a bodily fluid. Bodily fluids include any fluid produced by the body that may contain a target analyte. Non-limiting examples of bodily fluids include sweat, blood, urine, lavage, plasma, saliva, tears, and so forth. The disclosures herein are not intended to be limited to detecting and quantitating analytes in any specific bodily fluid or subset of bodily fluids.

The present method and device can be customized for the detection of a variety of specific target analytes, including but not limited to: biomarkers to measure wellness (e.g. cortisol, glucose, lactate), indicators of certain bodily conditions, illnesses or diseases (e.g., enzymes, neurotransmitters), drugs of abuse, hormones and hormone-like compounds (e.g., estrogen and estrogen-like compounds, progesterone, pregnancy hormones, testosterone, growth hormones, adrenaline, steroids, cortisol, erythropoietin, angiotensin, etc.), calcium, cholesterol, triglycerides, blood sugars, caffeine, vitamins (e.g., A vitamins, B vitamins, vitamin C, D vitamins, and E vitamins), nutrients, supplements, alcohol, poisons, and toxins (e.g., snake, scorpion, etc.), and metabolites thereof (e.g., ethyl gluronide, a metabolite of alcohol). Drugs of abuse may include both prescription drugs (e.g., morphine, oxycodone, hydrocodone, amphetamines, barbiturates, benzodiazepines, gamma-hydroxybutyric acid (GHB), ketamine, etc.) and street drugs, including narcotics (e.g., heroin, dilaudid/hydromorphone, methadone, opioids, opiates, codeine, etc.), stimulants (e.g., methamphetamine, cocaine, methcathinone, etc.), hallucinogens (e.g., LSD, active ingredients in certain mushrooms such as psilocybin and psilocin), ecstasy, synthetic cannabis (K2), peyote, mescaline, cannabis, cannabinoids, cathinones, synthetic stimulants (e.g., “bath salts”), ambien/zolpidem, seroquel/quetiapine, desoxyn, adderall/mixed amphetamine salts, laudanum/tincture of opium, oxymorphone/opana, and others. Accordingly, it should be appreciated that any suitable target analyte may be analyzed using an analysis device described herein. In some embodiments, the target analyte is one that can be found in a bodily fluid sample (e.g., sweat, blood, urine, lavage, plasma, saliva, tears), although other fluid samples may be possible.

Certain examples of the disclosed method and device can be used to measure the level of drug impairment of the test subject. In such embodiments, the method(s) and device(s) can be used by law enforcement, health care personnel, treatment facilities, or others wishing to detect or monitor impairment and/or drug use.

As used herein, a “user”, “subject” or “test subject” refers to any mammal (e.g., a human), for example, a mammal for which a target analyte is to be determined. Examples of users or subjects include a human, a non-human primate, a cow, a horse, a pig, a sheep, a goat, a dog, a cat or a rodent such as a mouse, a rat, a hamster, or a guinea pig. Generally, the embodiments described herein are directed toward use with humans. A user or subject may be one this is suspected of having, known to have (e.g., diagnosed with), and/or at risk of developing a bodily condition. Determination of a target analyte may give an indication of the present/absence of the bodily condition, the progression of the bodily condition, and/or security of the bodily condition.

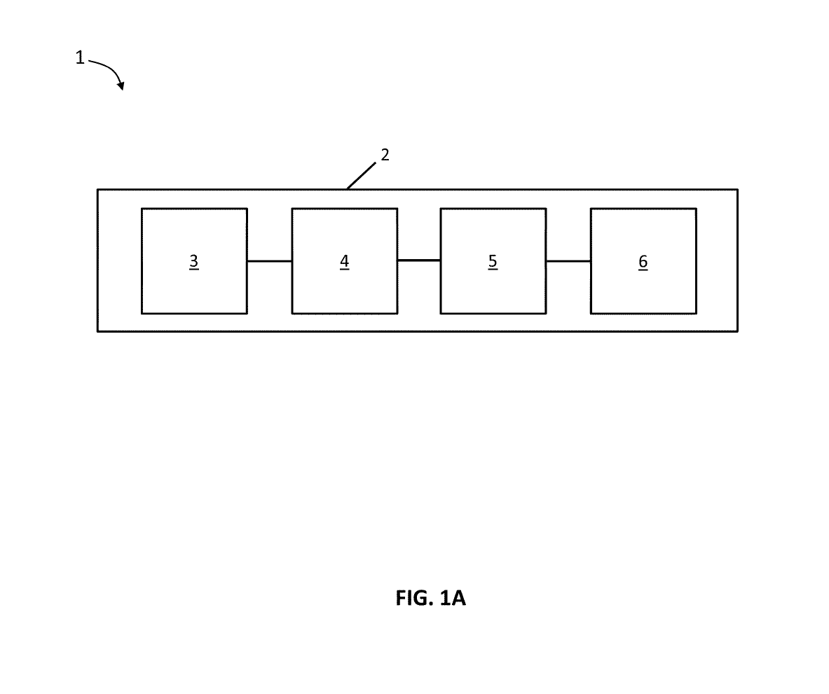

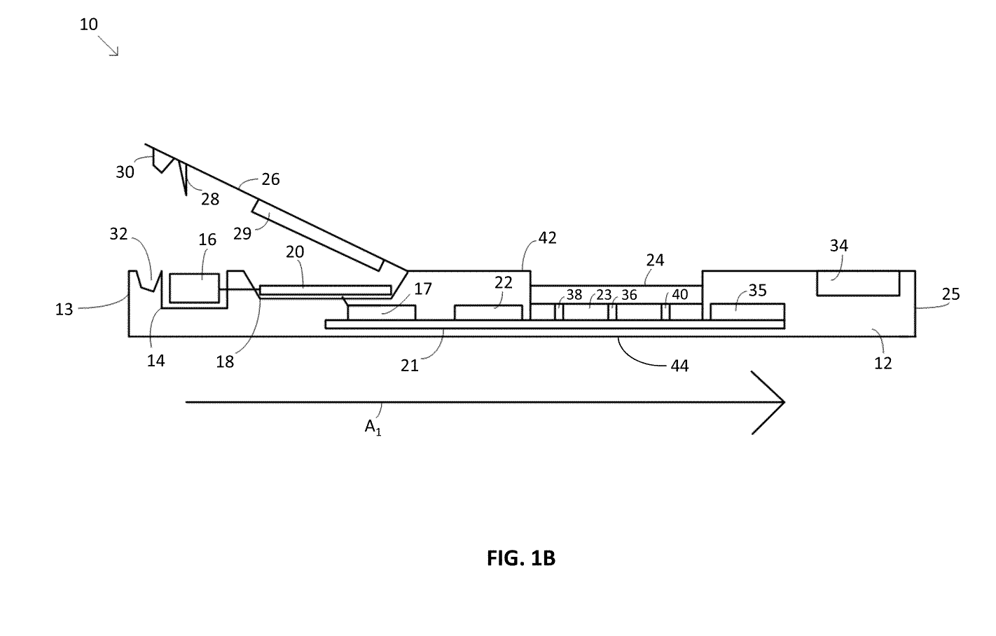

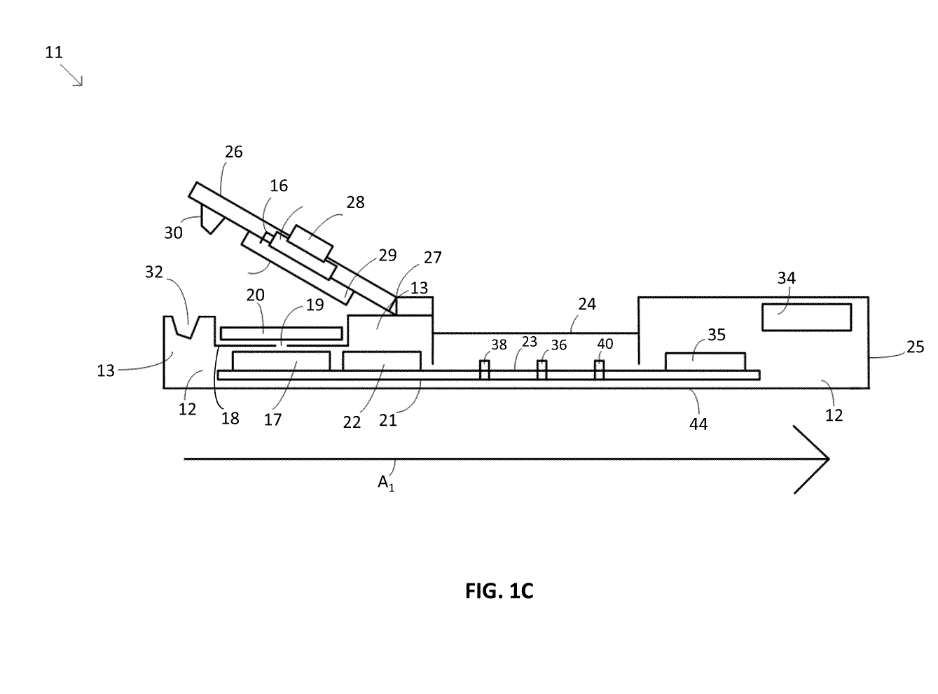

FIG. 1A is a schematic diagram representing an analysis device according to certain embodiments described herein. As shown illustratively in FIG. 1A, an analysis device (e.g., a bodily fluid analysis device) may include a device body 2 including a liquid reservoir 3 that may contain a liquid such as a buffered solution or any other suitable fluid, e.g., for eluting a sample (e.g., bodily fluid sample). The bodily fluid analysis device may also include a region 4 adapted and arranged for housing a test sample element (e.g., an absorbent material) and/or a sample. For instance, region 3 may be a nesting region for holding a test sample element in the form of an absorbent pad comprising an absorbent material used to collect a sample (e.g., bodily fluid sample). In some cases, the test sample element (e.g., absorbent material) may be integrally (e.g., irreversibly) connected to the device, such as in embodiments in which the bodily fluid analysis device is designed to be worn by a user. In other cases, the test sample element (e.g., absorbent material) may be reversibly connected to the device, such as in embodiments in which the test sample element (e.g., absorbent material) is used to collect a sample (e.g., bodily fluid sample) and then inserted into the device for analysis of the sample.

When the bodily fluid analysis device contains a sample, the liquid may be released from the liquid reservoir into the absorbent material. The liquid may mix with the sample (e.g., bodily fluid sample) and flow into a conjugate region 5. The conjugate region may comprise one or more conjugates that may bind with one or more target analytes and/or one or more non-target analytes. Binding of a target analyte and/or non-target analyte with one or more conjugates may produce one or more target analyte-conjugate complexes for one or more non-target analyte-conjugate complexes. The complexes may flow along a test strip 6 and may be captured at one or more test bands within the test strip. Determination of the amount of complexes captured at the test bands can be used to determine the presence, an amount, and/or a concentration of the target analyte in the sample (e.g., bodily fluid sample).

As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, or separating components fastened together via adhesives or tools. Devices including features forming an irreversible connection may be useful, for example, for one-time-use (e.g., disposable) devices. Such devices may form an irreversible connection so that the user cannot interfere with a chemical and/or biological binding interaction being performed in the device after connection.

It should be appreciated that while FIG. 1A and other figures provided herein show certain components of a device, not all components need be present in all embodiments described herein. Moreover, the configuration of components shown in the figures may vary. For example, while a conjugate region may be positioned downstream of the region adapted and arranged for housing a test sample element and/or sample, in some embodiments the conjugate region may be positioned upstream, or above or below, such a region. Additionally, other components not shown in the figures may be present in certain embodiments. For example, in some cases a wicking element is positioned downstream of the test strip to aid fluid flow in the device. In another example, an absorption element may be positioned adjacent and/or downstream of the test sample element (e.g., between the test sample element and a conjugate region) to aid the flowing of sample and/or buffer towards a downstream end of the device. Other configurations are also possible.

Accordingly, in some embodiments an analysis device (e.g., a bodily fluid analysis device) for determining a target analyte in a sample (e.g., bodily fluid sample) comprises a liquid reservoir containing a liquid, wherein the liquid reservoir is sealed prior to use and is adapted and arranged to release the liquid during use. The device may also include a region adapted and arranged for housing a test sample element containing a sample, wherein the region is positioned downstream of the liquid reservoir. The device may also include a conjugate region comprising at least a first conjugate adapted and arranged to bind to the target analyte, wherein the conjugate region is positioned downstream of the liquid reservoir. The device may also include a lateral flow assay, the lateral flow assay comprising of a test strip that comprises a first band adapted and arranged to capture the target analyte. A method of determining a target analyte in a sample (e.g., bodily fluid sample) may comprise introducing the sample comprising the target analyte into a region of an analysis device (e.g., a bodily fluid analysis device), wherein the bodily fluid analysis device comprises a lateral flow assay and a sealed liquid reservoir containing a liquid. The method may involve releasing the liquid from the liquid reservoir, flowing the liquid across the region containing the sample (e.g., bodily fluid sample) comprising the target analyte and allowing the target analyte from the sample to bind with a first conjugate to form a target analyte-conjugate complex. The method may involve flowing the target analyte-conjugate complex across a test strip, capturing the target analyte-conjugate complex at a first band of the test strip, and determining the target analyte.

As used herein, “prior to use” of a device or a component of a device means a time or times before the device or component is first used by an intended user after commercial sale. Use may include any step(s) requiring manipulation of the device by a user. For example, use may involve one or more steps such as removing a seal or cover from the device, collecting a sample using a test sample component, introducing a sample into the device, performing a reaction/analysis with a sample, detection of a sample, etc. Use, in this context, does not include manufacture or other preparatory or quality control steps taken by the manufacturer of the device. Those of ordinary skill in the art are well aware of the meaning of use in this context, and will be able easily to determine whether a device described herein has or has not experienced use. In one set of embodiments, a device described herein is disposable after use (e.g., after completion of an assay), and it is particularly evident when such devices are used, because it is typically impractical to use the device at all (e.g., for performing a second assay) after its use.

Although any suitable fluid such as a bodily fluid may be analyzed using the methods and devices described herein, in some embodiments, the methods and devices of the present disclosure are configured to detect and/or quantitate one or more target analytes in sweat sample (e.g., a human sweat sample). Sweat can contain numerous and wide-ranging analytes that are present in the body, including but not limited to drugs and their metabolites, physiological indicators, vitamins, nutrients, alcohol, and other substances. The present disclosure provides for methods and devices that can be used for obtaining and processing a sample of sweat for the purpose of determining the presence and/or quantitation of such analytes in the body. In some embodiments, the methods and devices may involve obtaining and processing a sample of sweat for the purpose of determining impairment (e.g., drug impairment) as defined by the concentration of analyte present in the sweat sample.

Many compounds in the body, including those present in blood or urine, are also excreted in or diffused into sweat. Sweat is produced by eccrine and apocrine glands in the skin to help maintain a constant core body temperature. The amount of sweat secreted varies based on an individual's physiological differences, activity, emotional state, environment, and other factors. During vigorous activity, adults can sweat up to 2-4 liters per hour under certain conditions. However, during low activity in a cool environment, sweat production can be minimal. Sweat is not produced evenly throughout the body, but rather sweat glands are unevenly distributed, with the highest concentration of eccrine sweat glands located in the palms of the hands.

Sweat is mainly composed of water and electrolytes, such as sodium, chloride, potassium, magnesium, urea, lactate, amino acids, bicarbonate, and calcium. A small fraction of sweat, about 1%, consists of other components, such as proteins, glycoproteins, lipids, and other compounds that are also found in blood, including drugs and their metabolites. Various physiological molecules and indicators, such as blood sugar and cholesterol, as well as drugs and their metabolites may also be present in sweat.

Immunological test methods (e.g., immunoassays) are generally used to test for the presence or absence of an analyte/antigen by taking advantage of the reaction between an analyte and an analyte/antigen-specific antibody. Various arrangements of test devices, antibodies, antigens, and detection tags can be used. For example, a target antibody or antigen can be bound to a visually detectable tag, such as a color developing molecule or fluorescent marker. Alternatively, an enzymatic tag can be used that binds to the analyte. The antibody or antigen can further be bound to a surface of a test device; such as a test strip or test well. Additionally, the immunoassay can be competitive or direct (i.e., noncompetitive, also known as a sandwich assay), and can measure either the target analyte or its antibody in the subject sample.

Lateral flow tests are often used in immunoassays to test for the presence or absence of a target analyte, and are typically arranged on a test strip that can be used without additional analytical equipment. For example, home pregnancy tests commonly use a lateral flow strip that tests for the presence or absence of certain hormone levels indicative of pregnancy. In some lateral flow tests, a fluid sample is applied to an absorbent portion of a test strip. The fluid then migrates across the strip toward a region containing a conjugate that binds to the analyte, forming an analyte-conjugate complex. This analyte-conjugate complex then migrates further along the strip toward a band of immobilized molecules that bind the complex. The conjugate may be labeled or tagged so that the band exhibits a detectable indicator (e.g., a color change) when a threshold concentration of complexes binds to the immobilized molecules. In a competitive assay, the target analyte and conjugated analyte molecules compete for binding locations on the test strip. The test strip may also include a control band to document that the test reaction has gone to completion.

In at least one embodiment, a method involves obtaining a sweat sample from the surface of a subject's skin and analyzing the sample using an immunological or chemical method in order to determine the presence of a target analyte. The sample may be obtained from the surface of a subject's skin using an absorbent region/material of a sweat collection device. Any suitable amount of sweat may be collected for analysis. For instance, in certain embodiments, sweat in the range of from 50 μL to 500 μL is collected for analysis. Other suitable volumes are described in more detail below. Once the sweat has been absorbed onto the absorbent region/material, a liquid reservoir component of the device may be used to release a buffer that elutes the analyte from the absorbent region. In some embodiments, the buffer includes a volumetric analyte, i.e. a detectable analyte of known concentration in the buffer. In some embodiments, an optional wicking element on the device draws the eluted analyte by wicking action toward a conjugate region on the device, and then toward an analysis region on the device. In some cases, the method can also include a step of inducing sweating in the subject in order to generate an adequate amount of sweat for analysis. For example, sweat can be induced by thermal pretreatment, electrical pretreatment, chemical pretreatment, and combinations thereof. Sweat may also be induced by exercise, as performed by the subject. In other embodiments, other embodiments, other bodily fluid samples besides sweat can be used.

In certain embodiments, a device described herein includes an immunological assay that utilizes an antibody specific to a target analyte. In certain embodiments, a device may be configured as a chemical assay. Chemical assays are suitable for analytes that do not readily lend themselves to immunological methods because, for example, the analyte is non-immunogenic and therefore lacks available antibodies. Examples of such analytes include alcohol, calcium, and blood sugars. Other types of analyses are also possible.

FIG. 1B is a schematic cross-sectional side view representation of an analysis device (e.g., a bodily fluid analysis device) according to at least one embodiment of the present disclosure. As shown illustratively in FIG. As illustratively 1B, an analysis device 10 includes a device body 12, a first end 13, a region 14 for holding a liquid reservoir 16 containing a buffered solution (or other suitable liquid) initially contained within the liquid reservoir (e.g., prior to use), and an absorption element 17.

As shown illustratively in FIG. 1B, the device also includes a region 18 (e.g., holding region) which may be used to house a test sample element (e.g., an absorbent material) and/or sample. A test sample element 20 such as an absorbent material may be used to introduce the sample into the device. Absorption element 17 (if present) may absorb at least a portion of the sample to draw the sample towards a downstream end of the device. The device may also include a lateral flow assay 21 including a conjugate region 22, a test strip 23, an optional results window 24, and a second end 25.

As shown illustratively in FIG. 1C, the absorption element may be positioned adjacent (e.g., beside) the test sample element and the conjugate region. Other configurations of the absorption element are also possible, as described herein.

In some embodiments, the device also includes a lid 26. In some embodiments, the lid includes a liquid releasing member 28 (e.g., a puncture component). In certain embodiments, the lid includes a compression element 29 that may be used to compress all or portions of the test sample element. The lid may optionally include a first locking member 30 and the device body may optionally include a second locking member 32 (e.g., which may be complementary to the first locking member), e.g., for securing the lid to the body portion of the device.

In some embodiments the device includes a device identifying component 34 (e.g., a RFID tag, a barcode, a QR code, a Bluetooth-enabled component, or a Wi-Fi-enabled component). The device may also include a test strip 23, an optional wicking element 35, a first band 36 (e.g., a test band), and/or a second band 40 (e.g., control band). In some embodiments, the test strip also includes a third band 38 (e.g., a volume band). The analysis device may also include a top 42 and a bottom 44.

The terms “first”, “second”, “third”, etc., as used herein generally refer to separate embodiments, portions, entities or components of a device. For instance, a “first band” is separate from a “second band”, though they may have the same or different configuration or composition. For instance, in some cases a first band is a test band, and a second band is another test band. In other embodiments, a first band is a test band, and a second band is a non-target analyte band. The terms “first” and/or “second” do not necessarily denote a particular order of the embodiments, portions, entities or components on the device. For example, in some embodiments a first band (e.g., a test band) may be positioned upstream of a second band (e.g., control band) and/or a third band (e.g., a volume band). In other embodiments, a third band may be positioned upstream of a first band and/or a second band. Other configurations are also possible.

Another configuration of an analysis device (e.g., a bodily fluid analysis device) is shown in FIG. 1C. As shown illustratively in FIG. 1C, an analysis device 11 includes a device body 12, a first end 13, a liquid reservoir 16 containing a buffered solution (or other suitable liquid) initially contained within the liquid reservoir (e.g., prior to use), and an absorption element 17. As shown illustratively in FIG. 1C, the device also includes a region 18 (e.g., holding region) which may be used to house a test sample element (e.g., an absorbent material) and/or sample. A test sample element 20 such as an absorbent material may be used to introduce the sample into the device. The illustrative device may also include a lateral flow assay 21 including a conjugate region 22, a test strip 23, a results window 24, and a second end 25.

In embodiments in which the device includes absorption element 17, the absorption element may be positioned adjacent and/or downstream of the test sample element (e.g., between the test sample element and a conjugate region) to aid the flowing of sample and/or buffer towards a downstream end of the device. As shown illustratively in FIG. 1C, the absorption element may be positioned underneath the test sample element and beside the conjugate region. However, it should be appreciated that other configurations are also possible. For instance, in some cases the absorption element is combined with the conjugate region.

In some embodiments, the device also includes a lid 26. In some instances, the lid includes a liquid releasing member 28, which may optionally include a locking mechanism. The liquid releasing member may include any suitable component for releasing the liquid contained in the liquid reservoir during use. As described in more detail below, in some embodiments the releasing member includes a button that the user can compress to initiate release of the liquid from the liquid reservoir. The button may include a locking mechanism so that once the button is compressed, the button is maintained in a compressed state. Once a liquid (e.g., buffered solution, water) is released from the liquid reservoir, it may flow into test sample element 20 and combine with the sample fluid contained in the test sample element. A mixture of the liquid (e.g., buffered solution, water) and the sample fluid may flow into conjugate region 22 (e.g., via a conduit 19). Although FIG. 1C shows separation (e.g., a separator) between region 18 and conjugate region 22, as well as conduit 19 allowing fluid communication between the two regions, in other embodiments, no such separation may be present.

In some instances, the lid may include a hinge 27 to attach the lid to the body portion of the device. In certain embodiments, the lid includes a compression element 29 that may be used to compress all or portions of the test sample element. The lid may optionally include a first locking member 30 and the device body may optionally include a second locking member 32 (e.g., which may be complementary to the first locking member), e.g., for securing the lid to the body portion of the device.

In some embodiments the device includes a device identifying component 34 (e.g., a RFID tag, a barcode, a QR code, a Bluetooth-enabled component, or a Wi-Fi-enabled component). The device may also include a test strip 23, an optional wicking element 35, a first band 36 (e.g., a test band), and/or a second band 40 (e.g., control band). In some embodiments, the test strip also includes a third band 38 (e.g., a volume band). The analysis device may also include a top 42 and a bottom 44.

Accordingly, in some embodiments an analysis device (e.g., a bodily fluid analysis device) is used for determining a concentration of a target analyte in a sample (e.g., bodily fluid sample). The bodily fluid analysis device may comprise a lateral flow assay, the lateral flow assay comprising a test strip. The test strip comprises a first band and a second band, the first band adapted and arranged to determine a quantity of the target analyte in the sample (e.g., bodily fluid sample), and the second band adapted and arranged to determine a volume of the sample (e.g., bodily fluid sample). A method of determining a concentration of a target analyte in a sample (e.g., bodily fluid sample) may comprising, for instance, introducing the sample comprising the target analyte into an analysis device (e.g., a bodily fluid analysis device), wherein the bodily fluid analysis device comprises a lateral flow assay, and passing the sample across a test strip comprising a first band and a second band. The first band may be adapted and arranged to determine a quantity of the target analyte in the sample, and the second band may be adapted and arranged to determine a volume of the sample. The method also involves determining the concentration of the target analyte in the sample based on the steps of determining the quantity of the target analyte and the volume of the sample.

In certain embodiments, an analysis device (e.g., a bodily fluid analysis device) for determining a concentration of a target analyte in a sample (e.g., bodily fluid sample) is provided. The device comprises a lateral flow assay, the lateral flow assay comprising a non-target analyte and a test strip. The test strip comprises a first band and a second band, the first band adapted and arranged to determine a quantity of the target analyte in the sample, and the second band adapted and arranged to determine a quantity of the non-target analyte. A method of determining a concentration of a target analyte in a sample may comprise introducing the sample comprising the target analyte into an analysis device (e.g., a bodily fluid analysis device), wherein the bodily fluid analysis device comprises a lateral flow assay. The method may also involve passing the sample across a test strip comprising a first band and a second band, wherein the first band is adapted and arranged to capture the target analyte in the sample, and wherein the second band is adapted and arranged to capture a non-target analyte. The method involves determining the concentration of the target analyte in the sample based on the steps of capturing the target analyte and non-target analyte.

Additional configurations of bodily fluid analysis devices are described in more detail below.

As described herein, an analysis device (e.g., a bodily fluid analysis device) may include a device body. The device body may comprise or be formed of any suitable material. For example, in some embodiments the device body may comprise a polymeric material or a combination of polymeric materials, such as polyester, polystyrene, polyvinyl chloride (PVC), polyethylene, polypropylene, and combinations thereof. The choice of material for the device body may depend in part, in some cases, on the target analyte because different analytes have different hydrophobicities/hydrophilicies. For example, hydrophobic analytes such as delta9-tetrahydrocannabinol (THC), the active ingredient in cannabis, tend to cling to highly charged surfaces, in which case it may be more suitable to use a device body comprising a polymeric material with a neutral surface charge. It should be appreciated, however, that portions of the device body may be coated with a suitable coating in order to impart a desirable surface chemistry.

In some embodiments, the thickness of the device body may be in a range from about 0.05 mm-50 mm, although thicknesses outside of this range would also be suitable. For instance, the device body may have a thickness of at least 0.05 mm, at least 0.075 mm, at least 0.1 mm, at least 0.125 mm, at least 1.0 mm, at least 2.0 mm, at least 5.0 mm, at least 10.0 mm, at least 20.0 mm, at least 30.0 mm, at least 40.0 mm, or at least 50.0 mm. In some instances, the device body has a thickness of less than or equal to 50.0 mm, less than or equal to 40.0 mm, less than or equal to 30.0 mm, less than or equal to 20.0 mm, less than or equal to 10.0 mm, less than or equal to 5.0 mm, less than or equal to 2.0 mm, less than or equal to 1.0 mm, or less than or equal to 0.5 mm. Combinations of the above-referenced ranges are also possible (e.g., at least 1.0 mm and less than or equal to 5.0 mm). Other ranges are also possible.

In some embodiments, the device body includes a multi-layered structure. For example, one or more layers may provide the device body with structural integrity, and/or one or more layers may provide the device body with a particular surface chemistry (e.g., neutral surface charge, positive surface charge, or negative surface charge). In certain embodiments, the device body includes a single-layered structure.

In some embodiments, all or portions of the device body comprise a material that is substantially non-permeable to water, aqueous liquids and/or vapors of aqueous-based liquids. For instance, all or portions of the device body may comprise a material having, for example, a water vapor permeability of less than or equal to 10.0 g·mm/m²·d, less than or equal to 8.0 g·mm/m²·d, less than or equal to 6.0 g·mm/m²·d, less than or equal to 5.0 g·mm/m²·d, less than or equal to 4.0 g·mm/m²·d, less than or equal to 3.0 g·mm/m²·d, less than or equal to 2.0 g·mm/m²·d, less than or equal to 1.0 g·mm/m²·d, less than or equal to 0.5 g·mm/m²·d, less than or equal to 0.3 g·mm/m²·d, less than or equal to 0.1 g·mm/m²·d, or less than or equal to 0.05 g·mm/m²·d. In some embodiments, the water vapor permeability may be, for example, at least 0.01 g·mm/m²·d, at least 0.05 g·mm/m²·d, at least 0.1 g·mm/m²·d, at least 1.0 g·mm/m²·d, at least 2.0 g·mm/m²·d, at least 3.0 g·mm/m²·d, at least 4.0 g·mm/m²·d, at least 5.0 g·mm/m²·d, or at least 8.0 g·mm/m²·d. Combinations of the above referenced ranges are also possible (e.g., at least between 0.01 g·mm/m²·d and less than or equal to 2.0 g·mm/m²·d). The water vapor permeability may be measured at, for example, 40° C. at 90% relative humidity (RH).

In some embodiments, a device described herein may include a liquid reservoir for containing a liquid (e.g., buffered solution, water) to be used in an analysis carried out by the device. The liquid reservoir may include, in some embodiments, a cavity or depression disposed in the device body. The liquid reservoir may be sealed prior to use of the device. For instance, in some embodiments the liquid (e.g., buffered solution, water) contained within the reservoir is initially hermetically sealed off from the rest of the analysis device until the analysis device is ready to be used to analyze a sample of bodily fluid.

Any suitable seal may be used (e.g., a foil, a polymeric seal, pliable plastic such as polyethylene or polypropylene, etc.) to form all or portions of the liquid reservoir. In some embodiments, the liquid (e.g., buffered solution, water) is contained in the liquid reservoir in the form of an initially sealed blister pack (e.g., sealed prior to use of the device). In some instances, the blister pack may be removably positioned in a region of the device. In some instances, the liquid reservoir (e.g., blister pack) may include a portion (e.g., a cover layer, a breakable portion, a frangible portion) that can be broken, pierced, or punctured to release a liquid from the reservoir. In some cases a first portion of the liquid reservoir has a smaller thickness than a second portion of the liquid reservoir such that application of pressure to the liquid reservoir causes breakage of the liquid reservoir at the first portion. It should be appreciated, however, that the liquid (e.g., buffered solution, water) contained in the liquid reservoir may be released from the liquid reservoir in any suitable manner.

The choice of liquid or buffered solution may depend in part on, for example, the hydrophobicity/hydrophilicity of the target analyte. Suitable buffers may include, for example, aqueous solutions of one or more salts, sugars, proteins, surfactants, polymers, and combinations thereof. For example, the buffer may comprise phosphate-buffered saline (PBS) or a combination of NaCl, KCl, Na₂HPO₄, KH₂PO₄, and optionally CaCl₂ or MgCl₂. In some embodiments, the buffered solution comprises a phosphate (e.g., 10-200 mM phosphate), a protein for non-specific binding (e.g., 0.1-3% (wt:vol) bovine serum albumin (BSA)), a detergent (e.g., 0.01-1% (vol:vol) Tween 20), and/or a polymer (e.g., 0.1-2% (vol:vol) PVA). In particular embodiments, the buffered solution comprises about 50 mM phosphate, about 1% (wt:vol) BSA, about 1% (vol:vol) Tween 20, and about 0.5% (vol:vol) PVA. In some embodiments the buffered solution includes tris(hydroxymethyl)aminomethane (“tris”) and/or urea. For example, a tris buffer may be selected for detecting a target analyte that is THC, which is a highly hydrophobic molecule. In one example, the tris buffer comprises 25-100 mM tris, 1-6 M urea, 0-50 mM tricine, 0-10 mM EDTA, and 0-0.1% (vol:vol) Tween 20. The concentrations and ranges of concentrations provided in the above examples should not be deemed limiting, as suitable concentrations of solutes outside of these ranges may also be used for the buffered solution. In selecting the buffered solution, its compatibility with the immunological test may also be considered, as well as user safety when the buffered solution comes in contact with human skin or in the event that the liquid reservoir is compromised during use.

In some embodiments, the liquid reservoir contains a liquid such as water, aqueous solutes such as dilute acids and bases, and combinations thereof. In some cases the liquid may be present in the liquid reservoir prior to use of the device and/or prior to introduction of the sample into the device. In some cases, the liquid may be combined with one or more buffer components after the liquid has been released from the liquid reservoir. Suitable buffer components include, for example, salts, such as phosphates, carbonates, sugars (e.g., glucose, sucrose, fructose), surfactants (e.g., Tween 20), proteins (e.g., proteins contained in BSA), non-target analytes, polymers (e.g., PVA), and combinations thereof. The one or more buffer components may be in solid, particulate, and/or dried form and may be stored in a region of the device. For instance, the buffer components may be present on or in a component such as a buffer pad, test sample element, conjugate pad, or any other suitable component that includes the one or more buffer components. Accordingly, a buffered solution may be formed during use of the device by the mixing of the liquid initially contained in the liquid reservoir and the one or more buffer components.

In some embodiments, the buffer can be selected to improve solubility, elution, preservation, and/or analysis of the target analyte. In certain embodiments, the buffered solution is selected to adjust pH to facilitate detection of the target analyte by the analysis device. For example, the pH of sweat can be as low as 5, but the optimal pH for antibody/analyte reactions may be in a range of about 7-8. Thus, when the analysis device is used to detect and quantitate one or more analytes in sweat, an alkaline buffered solution may be selected to compensate for this pH difference. In some embodiments, the buffered solution may additionally or alternatively be used to disrupt hydrophobic interactions between analytes and components of the analysis device. In some embodiments, a buffered solution is selected that is capable of selectively eluting the target analyte.

In general, the liquid (e.g., buffered solution, water) contained in the liquid reservoir may any suitable pH. For example, the liquid (e.g., buffered solution, water) may have a pH of at least 6.5, at least 6.7, at least 6.8, at least 7.0, at least 7.1, at least 7.2, at least 7.3, at least 7.4, at least 7.5, at least 7.6, at least 7.8, at least 8.0, at least 8.2, or at least 8.5. The liquid may have a pH, in some instances, of less than or equal to 8.5, less than or equal to 8.3, less than or equal to 8.0, less than or equal to 7.8, less than or equal to 7.6, less than or equal to 7.5, less than or equal to 7.4, less than or equal to 7.3, less than or equal to 7.2, less than or equal to 7.0, less than or equal to 6.8. Combinations of the above-referenced ranges are also possible (e.g., at least 7.2 and less than or equal to 7.4). Other ranges are also possible.

The volume of the liquid reservoir and/or the volume of liquid (e.g., buffered solution, water) contained in the liquid reservoir can vary as desired. In some embodiments, the volume of the liquid reservoir and/or the volume of liquid (e.g., buffered solution, water) contained in the liquid reservoir may be at least 10 μL, at least 50 μL, at least 100 μL, at least 150 μL, at least 175 μL, at least 200 μL, at least 300 μL, at least 400 μL, at least 500 μL, at least 800 μL, or at least 1000 μL. In certain embodiments, the volume of the liquid reservoir and/or the volume of liquid (e.g., buffered solution, water) contained in the liquid reservoir may be less than or equal to 2000 μL, less than or equal to 1000 μL, less than or equal to 500 μL, less than or equal to 400 μL, less than or equal to 300 μL, less than or equal to 200 μL, less than or equal to 100 μL, less than or equal to 50 μL, or less than or equal to 25 μL. Combinations of the above-referenced ranges are also possible (e.g., at least 10 μL and less than or equal to 1000 μL, at least 200 μL and less than or equal to 500 μL). Other values and ranges are also possible.

In embodiments in which the liquid (e.g., buffered solution, water) contained in the liquid reservoir includes a detergent, the detergent may be present in any suitable amount. For instance, a detergent may be present in the liquid in an amount (% vol:vol) of at least 0.001%, at least 0.005%, at least 0.01%, at least 0.05%, at least 0.1%, at least 0.5%, at least 1%, at least 1.5%, at least 1%, at least 2.5%, at least 3%, at least 3.5%, at least 4%, or at least 4.5%, In some cases, a detergent may be present in the liquid in an amount (% vol:vol) of less than or equal to 5%, less than or equal to 4.5%, less than or equal to 4%, less than or equal to 3.5%, less than or equal to 3%, less than or equal to 2.5%, less than or equal to 2%, less than or equal to 1.5%, less than or equal to 1%, less than or equal to 0.5%, less than or equal to 0.1%, less than or equal to 0.005%. Combinations of the above-referenced ranges are also possible (e.g., 0.01% and 1%). Other ranges are also possible. If multiple detergents are present, each detergent may independently be present in one or more of the above-referenced ranges.

In embodiments in which the liquid (e.g., buffered solution, water) contained in the liquid reservoir includes a component for reducing non-specific binding (e.g., a protein, a polymer), the component may be present in any suitable amount. For instance, the component may be present in the liquid in an amount (% vol:vol) of at least 0.001%, at least 0.005%, at least 0.01%, at least 0.05%, at least 0.1%, at least 0.5%, at least 1%, at least 1.5%, at least 1%, at least 2.5%, at least 3%, at least 3.5%, at least 4%, or at least 4.5%, In some cases, the component may be present in the liquid in an amount (% vol:vol) of less than or equal to 5%, less than or equal to 4.5%, less than or equal to 4%, less than or equal to 3.5%, less than or equal to 3%, less than or equal to 2.5%, less than or equal to 2%, less than or equal to 1.5%, less than or equal to 1%, less than or equal to 0.5%, less than or equal to 0.1%, less than or equal to 0.005%. Combinations of the above-referenced ranges are also possible (e.g., 0.01% and 1%). Other ranges are also possible. If multiple components are present, each component may independently be present in one or more of the above-referenced ranges.

In some embodiments, a liquid (e.g., a buffered solution), which may be stored in the device prior to use (e.g., in the liquid reservoir), contains a known concentration of a non-target analyte. The non-target analyte may be selected such that it does not react with the bodily fluid being analyzed. In some embodiments, the non-target analyte is known to be absent from the bodily fluid being tested and/or is known to be immobilized by a lateral flow assay proportional to its concentration. The non-target analyte may be used, for example, as a volumetric analyte in the lateral flow assay, as described herein.

In some embodiments, the non-target analyte has at least one binding partner, i.e., a molecule that can undergo binding with the non-target analyte. Biological binding partners are examples; for instance, Protein A is a binding partner of the biological molecule IgG, and vice versa. Likewise, an antibody is a binding partner to its antigen, and vice versa.

In some embodiments, the concentration of the non-target analyte (e.g., volumetric analyte) in the buffered solution is known, and therefore functions as a de facto measurement of sample (e.g., bodily fluid sample) volume after the buffered solution mixes with the sample being tested. For example, the concentration of the non-target analyte (e.g., volumetric analyte) will decrease when the buffered solution mixes with sample that does not contain the non-target analyte. The volume of the sample can be deduced from the decrease in concentration of the non-target analyte in the mixture as compared with the concentration in the buffer alone.

In some embodiments the non-target analyte is a protein. For example, the non-target analyte may be immunoglobulin from a variety of species, including but not limited to albumin, biotin, avidin, casein, PHA, KLH, or another protein antigen that is easily detected using available reagents in a lateral flow assay. Other non-target analytes, including non-protein based analytes, can also be used such as aptamers, magnetic colored particles or molecular beacons.

As described herein, an analysis device (e.g., a bodily fluid analysis device) may include a region (e.g., region 18) configured to hold a test sample element (e.g., an absorbent material) and/or sample of bodily fluid being tested by the analysis device. In some embodiments, the region is a cavity or depression in the device body of the analysis device. In certain embodiments, to initiate a bodily fluid analysis using the analysis device, the sample (e.g., bodily fluid sample) and/or the test sample element containing the sample, is placed in the holding region. In particular embodiments, the test sample element is integrally connected to the body of the device.

In some embodiments, the sample (e.g., bodily fluid sample) is absorbed in the test sample element. For instance, the test sample element may be an absorbent pad/material that has absorbed (or adsorbed) a person's bodily fluid (e.g., sweat, blood, urine, lavage, plasma, saliva, tears) for testing. In some embodiments the sample (e.g., bodily fluid sample) is still wet when the test sample element is placed in the holding region. In other examples, the bodily fluid is allowed to partially or completely dry within the test sample element before the test sample element is introduced to the region, the target analyte being adsorbed by, or otherwise contained in or on, the test sample element. The region and the liquid reservoir may be configured and arranged to be in fluid communication with each other such that when the buffered solution is released from the liquid reservoir, the buffered solution migrates in the direction A₁ (FIGS. 1B and 1C) and makes fluid communication with the sample (e.g., bodily fluid sample) that has been placed in the holding region. In some embodiments, the buffered solution elutes the absorbed target analyte from the test sample element and continues to migrate (e.g., via capillary action) in the direction A₁ carrying the target analyte to the lateral flow assay.

In embodiments in which a device includes a non-target analyte (e.g., a volumetric analyte, a protein, or any other suitable component), the non-target analyte may be present in the device in any suitable amount. In some cases, a non-target analyte may present in an amount with respect to volume of the liquid contained in the liquid containment region (e.g., buffer). For instance, a non-target analyte may be present in the device in an amount of at least 0.1 μg per mL liquid, at least 0.5 μg, at least 1 μg, at least 5 μg, at least 10 μg, at least 20 μg, at least 50 μg, at least 100 μg, at least 200 μg, at least 500 μg, at least 1000 μg, at least 1500 μg, at least 2000 μg, at least 2500 μg, at least 3000 μg, at least 3500 μg, at least 4000 μg, or at least 4500 μg per mL liquid (μg/mL). In some instances, the non-target analyte may be present in the device in an amount of less than or equal to 5000 μg per mL liquid, less than or equal to 4000 μg, less than or equal to 3000 μg, less than or equal to 2000 μg, less than or equal to 1000 μg, less than or equal to 500 μg, less than or equal to 100 μg, less than or equal to 50 μg, less than or equal to 10 μg, or less than or equal to 1 μg per mL liquid (μg/mL). Combinations of the above-referenced ranges are also possible (e.g., at least 1 μg per mL liquid and less than or equal to 1000 μg per mL liquid (μg/mL)). Other ranges are also possible. If multiple conjugates are present (e.g., a target analyte conjugate and a non-target analyte conjugate), each conjugate may independently be present in one or more of the above-referenced ranges.

As described herein, in some embodiments a device includes an absorption element. The absorption element (e.g., absorption element 17) may be positioned adjacent and/or downstream of the test sample element (e.g., between the test sample element and a conjugate region), and/or upstream of the test strip, to aid the flowing of sample and/or buffer towards a downstream end of the device (e.g., towards the test strip). As shown illustratively in FIGS. 1B and 1C, the absorption element may be positioned underneath the test sample element and beside the conjugate region. However, it should be appreciated that other configurations are also possible. For instance, in some cases the absorption element is combined with the conjugate region. In other embodiments, at least a portion of the absorption element may be positioned on the same plane as the test sample element, a region containing the test sample element, the conjugate region, and/or the test strip.

Any suitable material can be used to form an absorption element, such as the materials described herein for the test sample element, wicking element, or conjugate region.

In some embodiments, the sample (e.g., bodily fluid sample) (and/or buffered solution) may flow across or through a conjugate region. In some embodiments, the conjugate region includes a pad or any suitable component/substrate containing one or more analyte conjugates. Such a conjugate pad may be any suitable shape, for example a rectangular shape, square shape, circular shape, etc. The conjugate region may include a conjugate selected to specifically bind to the target analyte. The conjugate may also be selected based on the analysis method used (e.g., an immunological method or a chemical assay). In some immunological examples, the conjugate is an antibody bound to a label (i.e., labeled). The conjugate may be labeled with any suitable type of label, including but not limited to an enzyme, a fluorescent label (e.g., a fluorescent nanoparticle), a chemiluminescent label, a colorimetric label, a bead, and/or a particle (e.g., nanoparticle) such as a gold particle. In some embodiments, the conjugate includes one or more nanoparticles, e.g., colloidal gold nanoparticles that are bound to an antibody.

In some embodiments, the conjugate region includes a conjugate pad on or in which a conjugate has been deposited on a pre-treated base layer of the pad made of any suitable material, such as glass fiber or a polymeric material (e.g., polyester), or rayon. In some cases, the conjugate region/pad may be composed of or comprise one or more materials described herein for the absorbent material and/or test strip, and/or may have one or more characteristics as described herein for the absorbent materials and/or test strip (e.g., average pore size). The base layer can be pre-treated before application of the conjugate by applying a solution onto the base layer and drying. The solution may comprise, by way of nonlimiting example, an aqueous solution of one or more salts, sugars, proteins, surfactants, and polymers. In an exemplary embodiment, the solution comprises about 10-200 mM phosphate, 0.2-3% bovine serum albumin (BSA), about 0.2-3% Tween 20, and about 0.1-2% polyvinyl alcohol (PVA). For example, the solution may comprise about 50 mM phosphate, about 1% BSA, about 1% Tween 20, and about 0.5% PVA.

The one or more conjugates may be associated with or attached to the conjugate region/conjugate pad in any suitable manner. For instance, in some cases the one or more conjugates may be absorbed or adsorbed to the conjugate region/conjugate pad. In certain embodiments, the one or more conjugates may be weakly bound to the conjugate region/conjugate pad (e.g., via hydrophobic interactions, hydrophilic interactions, van der Waals interactions, and/or electrostatic interactions). In some instances, the one or more conjugates may be positioned within a matrix (e.g., porous matrix, fibrous matrix, gel matrix) in the conjugate region. In some embodiments, the matrix is in the form of a membrane. In certain instances, the one or more conjugates may be absorbed to a surface of the conjugate region. Other configurations are also possible.

In some embodiments, the conjugate region includes one or more additional conjugates. For example, the conjugate region may include a non-target conjugate, such as a volumetric conjugate selected to bind to a volumetric analyte in the buffered solution. In some of these examples, the volumetric analyte includes an immunoglobulin that can be derived from a variety of species, albumin, biotin, avidin, casein, PHA, KLH, or another protein antigen. The volumetric analyte may have a corresponding binding partner used as the volumetric conjugate. If the volumetric analyte selected for the buffered solution is biotin, for example, a volumetric conjugate comprising streptavidin may be added to the conjugate region for the volumetric analyte to bind with.

In an alternative embodiment, the analysis device is configured as a chemical assay (rather than an immunological assay), wherein the conjugate pad and conjugate of the conjugate region of the lateral flow assay are replaced by a reagent region (e.g., reagent pad) and suitable reagents deposited on the reagent region/pad, respectively.

In embodiments in which a device includes a conjugate (e.g., a target analyte conjugate, a non-target analyte conjugate), the conjugate may be present in the device in any suitable amount. In some cases, a conjugate may present in an amount with respect to volume of the liquid contained in the liquid containment region (e.g., buffer). For instance, a conjugate may be present in the device in an amount of at least 0.1 μg per mL liquid, at least 0.5 μg, at least 1 μg, at least 5 μg, at least 10 μg, at least 20 μg, at least 50 μg, at least 100 μg, at least 200 μg, at least 500 μg, at least 1000 μg, at least 1500 μg, at least 2000 μg, at least 2500 μg, at least 3000 μg, at least 3500 μg, at least 4000 μg, or at least 4500 μg per mL liquid (μg/mL). In some instances, the conjugate may be present in the device in an amount of less than or equal to 5000 μg per mL liquid, less than or equal to 4000 μg, less than or equal to 3000 μg, less than or equal to 2000 μg, less than or equal to 1000 μg, less than or equal to 500 μg, less than or equal to 100 μg, less than or equal to 50 μg, less than or equal to 10 μg, or less than or equal to 1 μg per mL liquid (μg/mL). Combinations of the above-referenced ranges are also possible (e.g., at least 1 μg per mL liquid and less than or equal to 1000 μg per mL liquid (μg/mL)). Other ranges are also possible. If multiple conjugates are present (e.g., a target analyte conjugate and a non-target analyte conjugate), each conjugate may independently be present in one or more of the above-referenced ranges.

Several embodiments of bodily fluid analysis devices described herein may include a lateral flow assay. The lateral flow assay may be disposed within the device body. The lateral flow assay may be positioned downstream (i.e., further along the direction A₁) from the region (e.g., holding region) used to house a test sample element (e.g., an absorbent material) and/or sample (e.g., region 18 of FIGS. 1A and 1B). As described herein, in some embodiments, a mixture of the sample (e.g., bodily fluid sample) and buffered solution migrates into the lateral flow assay. In some embodiments, by the time the sample and buffered solution mixture reaches the lateral flow assay, the mixture includes a non-target analyte (e.g., volumetric analyte from the buffered solution) and/or a target analyte from the sample. In embodiments in which the buffered solution is not present in the device, the sample can migrate into the lateral flow assay, the sample optionally carrying with it one or more non-target analytes (e.g., volumetric analytes).

The lateral flow assay may include a test strip including one or more bands that may capture one or more analytes (e.g., target analytes, non-target analytes). The test strip may be positioned downstream (i.e., further along the direction A₁ in FIGS. 1B and 1C) from the conjugate region, reagent region, region for holding the absorbent material/sample, and/or the liquid reservoir. In some embodiments, the fluid sample containing the one or more analyte conjugate complexes may migrate or flow (e.g., via capillary action) to the test strip. In some embodiments, the test strip is an immunologic assay selected for a particular target analyte, such as a particular drug or other component. In some embodiments, the test strip is configured to provide a positive/negative (true/false) result (e.g., a qualitative result). In other embodiments, the test strip is configured to provide a quantitative result, as described in more detail herein. In some embodiments, the test strip includes a test band and a control band (e.g., test band 36 and control band 40 shown in FIGS. 1B and 1C). In some embodiments, the test strip includes a test band, a control band, and a volume band (e.g., volume band 38). In other examples, the test strip may comprise additional bands (e.g., additional test bands, control bands, and/or volume bands), or may comprise only a single test band.

The test strip may be composed of or comprise any suitable material(s), such as nitrocellulose or a membrane, such as CytoSep® available from Ahlstrom. CytoSep® is a single layer of highly pure natural and synthetic fibers that contains no chemical interfering substances and no significant binding of plasma components. In some embodiments, the material or materials used for the test strip may be porous, having an average pore size in a range from, e.g., 4 μm to about 50 μm. For example, the average pore size may be at least 4 μm, at least 6 μm, at least 8 μm, at least 10 μm, at least 15 μm, at least 20 μm, at least 25 μm, at least 30 μm, or at least 40 μm. In some cases, the average pore size may be less than or equal to 50 μm, less than or equal to 40 μm, less than or equal to 30 μm, less than or equal to 25 μm, less than or equal to 20 μm, less than or equal to 15 μm, less than or equal to 10 μm, or less than or equal to 8 μm. Combinations of the above-referenced ranges are also possible (e.g., at least 6 μm and less than or equal to 20 μm, at least 8 μm and less than or equal to 15 μm). Other ranges are also possible.

In some embodiments, the test strip comprises material(s) that facilitate movement of the fluid sample toward the test area. Such materials may be treated (e.g., with a coating) in order to improve wetting of the test strip and/or to minimize background noise.

Each analyte-conjugate complex flows in the direction A₁ along the test strip (FIGS. 1B and 1C). An example test strip including a volume band 38, a test band 36, and a control band 40, disposed in this order from the first end 13 and the second end 25 of the analysis devices shown in FIGS. 1B and 1C will now be described.

In certain embodiments in which the test strip includes a non-analyte target band such as a volumetric band (e.g., volumetric band 38), the fluid and/or particles flow, reaching the non-target (e.g., volumetric) band. The non-target (e.g., volumetric) band may include embedded or otherwise immobilized molecules known to bind the non-target (e.g., volumetric) analyte-conjugate complex. If the non-target (e.g., volumetric) analyte is present, which would be the case if the non-target (e.g., volumetric) analyte had been present in the buffered solution for example, the non-target (e.g., volumetric) band captures a portion of the non-target (e.g., volumetric) analyte-conjugate complex, preventing further migration thereof along the test strip. The non-target (e.g., volumetric) band may reveal an observable stripe or other observable marker left by the observable tag of the non-target (e.g., volumetric) analyte's conjugate. The amount of non-target (e.g., volumetric) analyte-conjugate complex captured by the non-target (e.g., volumetric) band 38 may be proportional to the concentration of the non-target (e.g., volumetric) analyte. In some embodiments, the non-target (e.g., volumetric) analyte is known not to be present in the sample (e.g., bodily fluid sample). A known quantity of the non-target (e.g., volumetric) may be present in the device (e.g., in the buffered solution) prior to use. The intensity of the non-target (e.g., volumetric) band may correlate (e.g., inversely or directly) with the volume of the sample. Accordingly, in some embodiments the amount of non-target (e.g., volumetric) analyte captured at this band can give an indication of the volume of the sample (e.g., bodily fluid sample) introduced into the device.

As an example, if the non-target (e.g., volumetric) analyte conjugate's detectable tag is fluorescent, the intensity of the non-target (e.g., volumetric) band can be measured, e.g., by measuring the amount of light emitted from the band when excited by a fluorimeter, the amount of emitted light correlating proportionally with a concentration of non-target (e.g., volumetric) analyte that has been captured by the non-target (e.g., volumetric) band. By extension, the amount of light emitted from the non-target (e.g., volumetric) band when excited by a fluorimeter may correlate (e.g., inversely or directly) with the volume of the sample (e.g., bodily fluid sample) tested in the lateral flow assay. If the non-target (e.g., volumetric) analyte conjugate's visually detectable tag is a color developing tag, such as a gold particle, the intensity of the non-target (e.g., volumetric) band can be measured using an optical reader sensitive to the darkness of the non-target (e.g., volumetric) band, the darkness of the non-target (e.g., volumetric) band correlating (e.g., inversely or directly) with the concentration of the non-target (e.g., volumetric) analyte.

The test band (e.g., band 36) may include embedded or otherwise immobilized molecules known to bind the target analyte-conjugate complex. If the target analyte is present in the bodily sample, the test band can capture the target analyte-conjugate complex, preventing further migration or flow thereof along the test strip. The test band may reveal an observable stripe or other visual marker left by the observably detectable tag of the target analyte's conjugate. Thus, in some embodiments, the intensity of the test band correlates proportionally with the amount of target analyte present in the sample. If the target analyte conjugate's detectable tag is fluorescent, the intensity of the test band can be measured, e.g., by measuring the amount of light emitted from the band when excited by a fluorimeter, the amount of emitted light correlating with the quantity of target analyte that has been captured by the test band. If the target analyte conjugate's observable tag is a color developing tag, such as a gold particle, the intensity of the test band can be measured using an optical reader sensitive to the darkness of the test band, the darkness of the test band correlating with quantity of target analyte that has been captured by the test band.

In some embodiments, the fluid and particles not captured by either test band or the non-target (e.g., volumetric) band continue to flow in the direction A₁ along the test strip until they encounter a control band. The control band may include embedded or otherwise immobilized molecules known to bind a free conjugate (e.g., one or both of a free target analyte conjugate and/or a free non-target (e.g., volumetric) analyte conjugate). Thus, in some such embodiments, the control band can capture one or more free conjugates eluted from the conjugate region that did not bind an analyte (target or non-target) in the conjugate region. In some cases, the control band may prevent further migration of such particles along the test strip. The control band may be adapted and arranged to reveal an observable stripe or other observable marker left by the detectable tag of the one or more conjugates. In certain embodiments, the amount of conjugate positioned in the conjugate region may generally be more than enough to bind all of the corresponding analyte in the bodily fluid/buffer. Therefore, in some scenarios, there may be free conjugate available for capture by the control band, which may be situated toward the second end (e.g., downstream end) of the analysis device. Revelation of the control band (via the detectable tags on the free conjugate captured by the control band) can indicate that the lateral flow assay functioned and worked to completion, i.e., it confirms the sample (e.g., bodily fluid sample) migrated across the conjugate region, test band, and/or non-target analyte (e.g., volumetric) band. By contrast, a control band that does not appear or have a change in property (e.g., color, fluorescence, intensity) may indicate a fault in the lateral flow assay, such as a problem with fluid migration along the assay, a problem of insufficient conjugate(s) at the conjugate region to bind the relevant analyte(s) in the fluid sample and so forth. Such evidence of a fault in the analysis may indicate that another analysis would have to be performed.

As described herein, in some embodiments a lateral flow assay of a device described herein includes a competitive assay. In a competitive assay, the target analyte in the sample and the molecules immobilized at the test band compete for the test analyte conjugate (initially positioned in the conjugate region). Thus, the greater amount of target analyte in the sample, the greater amount of binding between the target analyte and its conjugate, leaving less of the test analyte conjugate available for binding/capture at the test band. Accordingly, a lower intensity of the test band may indicate a higher amount/concentration of target analyte in the sample (i.e., the intensity of the test band is inversely correlated with the amount or concentration of target analyte in the sample).

Similarly, a volumetric test may also be a competitive assay, and can be used as part of a volumetric test to determine the volume of the sample collected using the test sample element. In this test, a known volumetric analyte in the buffer solution and the immobilized molecules at the volume band compete for the same volumetric analyte conjugate (initially positioned in the conjugate region). Thus, the greater the sample volume, the more dilute the sample/buffer mixture. There will be relatively less of the volumetric analyte present for binding with its conjugate, thereby decreasing the amount of volumetric analyte-conjugate available for binding/capture at the volume band (compared to if a smaller sample volume was present). Accordingly, a lower intensity of the volume band may indicate a higher volume of sample (i.e., the intensity of the volume band is inversely correlated with the sample volume).

In some embodiments, a lateral flow assay of a device described herein includes a sandwich assay. For example, an analyte (e.g., target analyte, non-target (e.g., volumetric) analyte) binds to a corresponding conjugate (e.g., conjugated antibody) disposed in the conjugate region. If the complex is a test analyte, the test analyte-conjugate complex flows towards the test band and can bind to a secondary antibody immobilized at the test band. The secondary antibody can be either an antibody that recognizes the target analyte-conjugated-antibody complex, or an antibody that recognizes the target analyte only. In either case, there will be no signal if the target analyte is not present.

Similarly, a non-target (e.g., volume) analyte can bind to a corresponding conjugate (e.g., conjugated antibody) disposed in the conjugate region to form a non-target (volumetric) analyte-conjugate complex. This complex may flow towards the non-target analyte (volumetric) band and can bind to a secondary antibody immobilized at the non-target analyte (volumetric) band. The secondary antibody can be either an antibody that recognizes the non-target (volumetric) analyte-conjugated-antibody complex, or an antibody that recognizes the non-target (volumetric) analyte only. In either case, there will be no signal if the non-target (volumetric) analyte is not present.

In alternative embodiments to the ones described above, the number, type, position and/or disposition of the bands can be varied. By way of non-limiting examples, there can be multiple test bands for capturing multiple target analytes, multiple control bands for different analytes (target and non-target), the non-target (e.g., volume) analyte band and/or control band can be eliminated, and/or the order/position of the bands can be varied. For instance, in one particular embodiment a non-target analyte (e.g., volume) band may be positioned downstream of a target analyte (test) band. Other configurations are also possible.

In some embodiments, an analysis device described herein includes a lid (e.g., lid 26 shown illustratively in FIGS. 1B and 1C). The lid may be attached to a portion of the device body in any suitable fashion (e.g., hingedly attached), for example, but may be attached to a top portion 42 of the analysis device (FIG. 1B). When the lid is in an open position (an example open position for the lid 26 is shown in FIGS. 1B and 1C) the test sample element 20 (e.g., an absorbent material) which may contain a sample to be analyzed, may be placed in the region for holding the test sample element (e.g., region 18).

In some embodiments the lid is sufficiently long (as measured along the direction of the arrow A₁) to cover both the liquid reservoir and the region 18 when the lid is in a closed position atop the analysis device (e.g., FIG. 1B). In certain embodiments, a liquid reservoir (e.g., liquid reservoir 16) is attached to a portion of the lid (e.g., as shown illustratively in FIG. 1C). The lid may include a movable portion (such as a button) that the user can compress to initiate release of the liquid from the liquid reservoir. The movable portion may include a locking mechanism in some embodiments. For example, a liquid releasing member (e.g., a puncture component) may be connected to the movable portion, and initiation of the movable portion may puncture a part of the liquid reservoir (e.g., one side, or two opposing sides of the reservoir). If a locking mechanism is present, the liquid releasing member may be maintained in a particular position or state that may help elute the fluid from the liquid reservoir completely. In some cases, the movable portion is adapted and arranged such that it cannot release liquid from the liquid reservoir until the lid is closed and/or locked into position. Other examples of liquid releasing members are provided below.

In some embodiments, the lid includes a first locking member (e.g., locking member 30 shown in FIGS. 1B and 1C) that mates with, or otherwise couples to, a second locking member disposed on the analysis device (e.g., second locking member 32). In some embodiments, the locking mechanism provided by the coupling of the first locking member and the second locking member is irreversible, i.e., the locking mechanism cannot be unlocked without destroying or damaging the analysis device. In this manner, the locking mechanism provided by the first locking member and/or the second locking member may prevent tampering with the analysis device or the bodily fluid test sample, e.g., once the test sample element containing the sample (e.g., bodily fluid sample) has been placed in the analysis device.

In some embodiments, the lid includes a compression element (e.g., compression element 29 shown in FIGS. 1B and 1C) configured to apply pressure, squeeze, and/or keep the test sample element in place when the lid is in the closed position. For example, the compression element can be one or more protrusions extending from the bottom of the lid. In some cases, the compression elements may protrude sufficiently from a suitable location on the lid to apply a desirable amount of pressure onto the test sample element when the lid is in a closed position, but not protruding too much to prevent closure of the lid. Alternatively, compression of the test sample element by the compression element can be controlled by the user after the lid has been closed. For example, the lid can include a movable portion (such as a button) that, when pressed towards the region containing the test sample element and/or sample (e.g., region 18), causes the movable portion of the lid to extend down into the region and apply pressure to the test sample element via the force applied to the movable portion of the lid. The compression element may be locked into a particular position in some embodiments. In some instances, controlled operation of the compression element may allow the user to wait until the buffer has been released and absorbed by the test sample element before compressing the test sample element. Compression of the test sample element containing the sample (e.g., bodily fluid sample) and the buffer mixture may, in some embodiments, enhance the migration of the mixture onto the lateral flow assay by squeezing the mixture out of the test sample element. Such compression may therefore minimize the amount of the mixture that is left behind in the test sample element. In addition, in some instances compression by the compression element may effect a mixing of the sample (e.g., bodily fluid sample) with the buffered solution to prepare a fluid sample of greater homogeneity. Alternatively, the analysis device may include a mixing element for enhanced mixing of the buffered solution with the sample (e.g., bodily fluid sample) prior to the mixture's flow onto the lateral flow assay.

In some embodiments, the device body or lid includes a liquid releasing member (e.g., liquid releasing member 28 shown in FIG. 1B). In certain configurations, the liquid releasing member may be attached to or otherwise positioned on the lid such that it engages the liquid reservoir when the lid is in a closed position. In some embodiments the liquid releasing member is a protrusion extending from the lid that enters or applies pressure to the liquid reservoir when the lid is in a closed position, thereby causing the release of the buffered solution. Alternatively, engagement of the liquid releasing member with the liquid reservoir can be controlled by the user after the lid has been closed. For example, the lid can include a movable portion (such as a button) that, when pressed towards the liquid reservoir causes the movable portion of the lid to extend down into the liquid reservoir, thereby engaging the moveable portion with the liquid reservoir, the engagement effecting the release of the buffer. Controlled operation of the buffer releasing element may allow the user to release the buffer at a desired moment following closure of the lid.

In some embodiments, the liquid releasing member doubles as a locking member that both releases the buffered solution and locks the lid in a closed position. This can be accomplished automatically upon closure of the lid, or alternatively in a controlled manner as just described.

In certain embodiments, a liquid releasing member is connected to a body portion of the device. The liquid releasing member may apply pressure to and/or puncture a portion of the liquid reservoir as described herein.

In some embodiments, a device described herein includes a wicking element. The wicking element (e.g., wicking element 35 shown in FIGS. 1B and 1C) may be positioned downstream of the test strip and/or may be in fluid communication with the test strip. The wicking element may comprise a wicking (e.g., absorbent) material that draws the buffer/bodily fluid mixture through the lateral flow assay. Suitable wicking materials for the wicking element may include, for example, non-synthetic/natural polymers (e.g., cellulose, high density cellulose, regenerated cellulose, cellulose acetate, cotton, wood pulp, hemp), synthetic polymers, and other natural or artificial absorbent materials. The wicking element may expedite the flow of the buffered solution and eluted target analyte and/or non-target (e.g., volumetric) analyte, and/or may prevent backflow toward the liquid reservoir. The wicking element may be of any suitable size and shape, such as rectangular, square, circular, etc.

It should be appreciated that in certain embodiments, a device described herein does not include a wicking element.

In some embodiments, a device described herein may include a device identifying component (e.g., device identifying component 34 shown in FIGS. 1B and 1C). The device identifying component may be active (e.g., requires a power source such as a battery to operate) or passive (i.e., it may contain information that can be read by a user or another device). In some embodiments, the device identifying component is attached (e.g., reversibly or irreversibly) to the outer surface of the analysis device. In some cases, the device identifying component may be integrated with the construction of the analysis device (e.g., integrally connected to a body portion of the device). The device identifying component may be adapted and arranged to identify the analysis device. For example, in some embodiments, the device identifying component identifies the particular analysis to be performed by the analysis device (e.g., THC quantitation in a sweat sample) or the particular type of sample to be used (e.g., sweat, blood, urine, lavage, plasma, saliva, tears). In some embodiments the device identifying component is configured to communicate with a lateral flow assay reader (discussed in more detail below) and/or is configured to instruct the reader regarding the analysis to be performed on the lateral flow assay. The device identifying components may be in electronic communication with a control system as described in more detail below. In some embodiments, the device identifying component is a RFID, a barcode, a QR code, a Bluetooth-enabled components, or a Wi-Fi-enabled component.

In one non-limiting embodiment, the device identifying component is a RFID, a barcode, a QR code, a Bluetooth-enabled components, a Wi-Fi-enabled component, or other suitable component, that holds device-related data in TAB format. In some embodiments, the data held by the device identifying component may include, for example, test name information, lot number information, calibration information, and/or distributor/manufacturer information. For example, test name information can include information about the nature of the test (e.g., cocaine, cannabis). In some embodiments, the lot number includes a unique product serial number. In some embodiments, the product serial number includes information about the nature of the test the analysis device is to be used for, the manufacturing date of the analysis device, the place of manufacture, and/or a random serialized number. In some embodiments, the random serialized number resets for each manufacturing date. The distributor/manufacturer information, if present, identifies the distributor and/or manufacturer of the analysis device. In some embodiments, one or more of the test name information, lot number information, and distributor/manufacturer information is/are a sequence or sequences of alphanumeric digits. In one example, the lot number is a twenty-two digit alpha numeric sequence including three digits to identify the type of test (e.g. COC for cocaine; THC for cannabis); six digits for identifying the month, day and year of manufacture (e.g. 091615 for Sep. 16, 2015); three digits for identifying the country of manufacture (e.g. USA, CAN for Canada; EUR for Europe); nine digits constituting a random serialized number, and a single digit verifier (e.g., a modulus 10 check digit). In some embodiments, the identifying component is a SMARTRAC CIRCUS NFC™ (Smartrac N.V. Amsterdam, The Netherlands) RFID component that is adhered to the outer surface of, or otherwise integrated with, the analysis device. In certain embodiments, the device identifying component includes a script for running a particular analysis relating to the assay. For instance, the analysis may be a particular protocol to be run by a detector (e.g., reader). Other configurations of a device identifying component are also possible.

In some embodiments, an analysis device may include a detector or reader. The detector or reader may, for example, allow determination of one or more analytes in the sample (e.g., bodily fluid sample). A variety of determination (e.g., measuring, quantifying, detecting, and/or qualifying) techniques may be used. Determination techniques may include optically-based techniques such as light transmission, light absorbance, colorimetric, light scattering, light reflection and visual techniques. Optical detection may occur in the infrared range (e.g., 0.01-7×10⁻⁵ cm), visible range (e.g., 7×10⁻⁵-4×10⁻⁵ cm), ultraviolet range (e.g., 4×10⁻⁵-10⁻⁷ cm), or combinations thereof. Determination techniques may also include luminescence techniques such as photoluminescence (e.g., fluorescence), chemiluminescence, bioluminescence, and/or electrochemiluminescence. In other embodiments, determination techniques may measure conductivity or resistance. The detector or reader may be configured to include these or other suitable detection systems. Specific examples of optical detectors are described in more detail herein. For instance, in some embodiments, an OPTRICON CUBE-READER™ (opTricon GmbH, Berlin, Germany) optical reader can be used.

The detector/reader may make a determination (e.g., determine an analyte; take a reading or measurement) after any suitable time after an assay begins. In some cases, a determination is made a certain time after a sample is introduced into the device, after a lid is closed and/or locked into position, after a liquid reservoir is pierced/broken, after a liquid from the liquid reservoir combines with a sample, after a fluid containing the sample begins to flow onto the test strip, and/or after the observation of a control band. For instance, the detector may make a determination at least 1 min, at least 2 mins, at least 3 mins, at least 5 mins, at least 7 mins, at least 8 mins, at least 10 mins, at least 12 mins, at least 15 mins, at least 20 mins, at least 30 mins, at least 45 mins, at least 1 hour, at least 2 hours, at least 6 hours, or at least 12 hours after one or more such events. In some cases, the detector makes a determination less than or equal to 1 day, less than or equal to 12 hours, less than or equal to 6 hours, less than or equal to 2 hours, less than or equal to 1 hour, less than or equal to 45 mins, less than or equal to 30 mins, less than or equal to 20 mins, less than or equal to 15 mins, less than or equal to 10 mins, less than or equal to 8 mins, less than or equal to 5 mins, less than or equal to 3 mins, less than or equal to 1 mins after one or more such events. Combinations of the above-referenced ranges are also possible (e.g., at least 10 mins and less than or equal to 30 mins after one or more such events). Other ranges are also possible. In certain embodiments, the detector/reader may make multiple determinations, each determination being made in one or more of the above-referenced ranges.

In some embodiments, the detector/reader may be adapted and arranged to make a single determination for each band present (e.g., test band, volume band and/or control band).

In some particular embodiments, a device or device component shown in the figures, or described with respect to the figures (e.g., any one of FIGS. 1-23), optional including a detector/reader described herein, may be designed to be worn by a user. For instance, the device or device component may include an adhesive or any other suitable component for attaching the device or component to the user. For example, a medium-tack, pressure-sensitive adhesive, such as GL187 available from G&L Adhesives Research, Glen Rock, Pa., may be used. In some embodiments, acrylic pressure-sensitive adhesives, elastomers, and combinations thereof can be used. Other adhesives are known in the art can be used in certain embodiments described herein. Depending on the intended use of the device, the device or device component may comprise an adhesive that adheres to the skin only once, i.e., the device cannot be removed and re-attached, or may be designed to attach to the skin multiple times. In some cases, the device or device component may include a band (e.g., an arm band, a wrist band, a leg band, a chest band, a finger band) that can be secured to a portion of the body of the user.

Attachment of the device or device component can be achieved at any suitable location on the subject, such as an arm (e.g., upper arm, lower arm, or wrist), hand, finger, back, chest, forehead, leg, foot, etc. In embodiments in which the device is designed to collect a sweat sample, the chosen attachment location may be one that provides adequate sweat production. For example, in one embodiment, the device component is attached to the lower side of the wrist of a subject.

The device or device component may remain on the skin for as long as needed to collect an adequate sample. For example, the device or device component may be adapted and arranged to remain on the skin for at least 10 seconds, at least 30 seconds, at least 1 minute, at least 3 minutes, at least 5 minutes, at least 10 minutes, at least 15 minutes, at least 30 minutes, or at least 60 minutes, at least 1 day, at least 1 week, or at least 1 month. In some cases, the device or device component may be adapted and arranged to remain on the skin for up to about 2 hours, up to about 6 hours, up to about 12 hours, up to about 18 hours, or up to about 24 hours, up to 1 week, or up to 1 month. Combinations of the above-referenced ranges are also possible (e.g., at least 1 minute and up to 24 hours). The duration may depend on the subject and the intended use (e.g., immediate testing, monitoring over time, etc.).

In other embodiments, the one or more devices is/are designed to not be worn by the user. For instance, the device may be configured in a hand-held format. In some such embodiments, a sample may be applied directly to a test sample element (e.g., an absorbent pad), which may be integrally connected to a body portion of the device. For instance, a user may hold the device including the test sample element adjacent his/her body to collect a sample (e.g., bodily fluid sample). In other embodiments, the test sample element may be inserted into, or removably connected to, the device. For instance, the test sample element may be used to collect a sample (e.g., bodily fluid sample) from the user, and then may be positioned in a region or reservoir of the device during use. Other configurations are also possible.

In certain embodiments, certain portions of an analysis device described herein may be adapted and arranged to be worn by a user (e.g., a body portion, a test sample element), and other portions of the device (e.g., a detector/reader) are adapted and arranged to not be worn by a user.

As described herein, a device may include a test sample element which may be used to collect a sample (e.g., bodily fluid sample). In some cases, the test sample element may be integrally (e.g., irreversibly) connected to a portion of the device body, such as in certain embodiments in which the bodily fluid analysis device is designed to be worn by a user. In other cases, the test sample element may be reversibly attached to the device, such as in certain embodiments in which the test sample element is used to collect a sample (e.g., bodily fluid sample) and then inserted into the device for analysis of the sample. Examples of test sample elements are now provided.

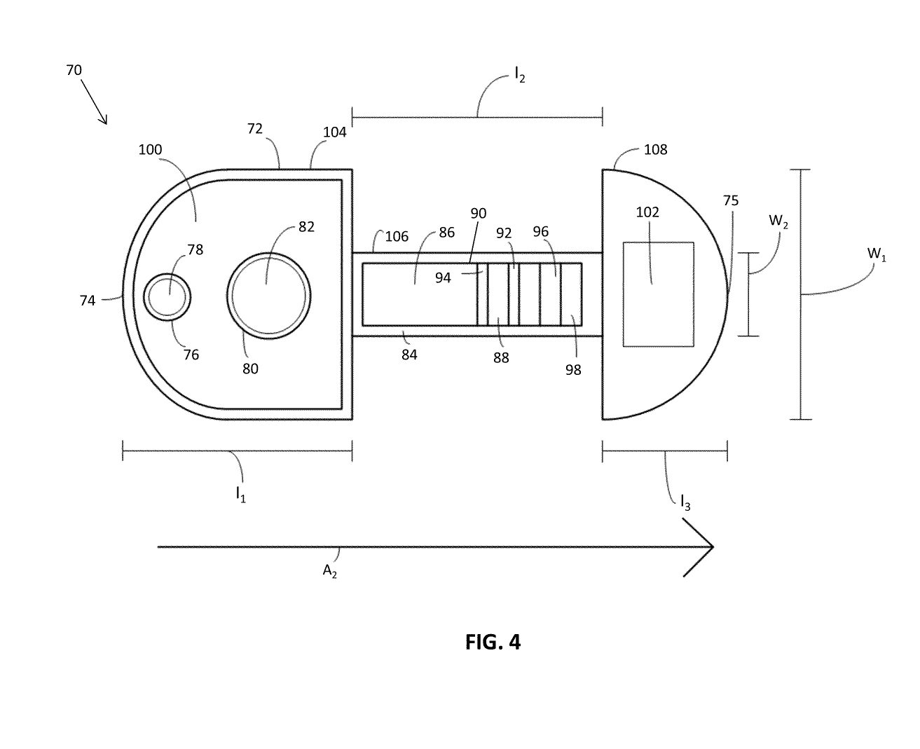

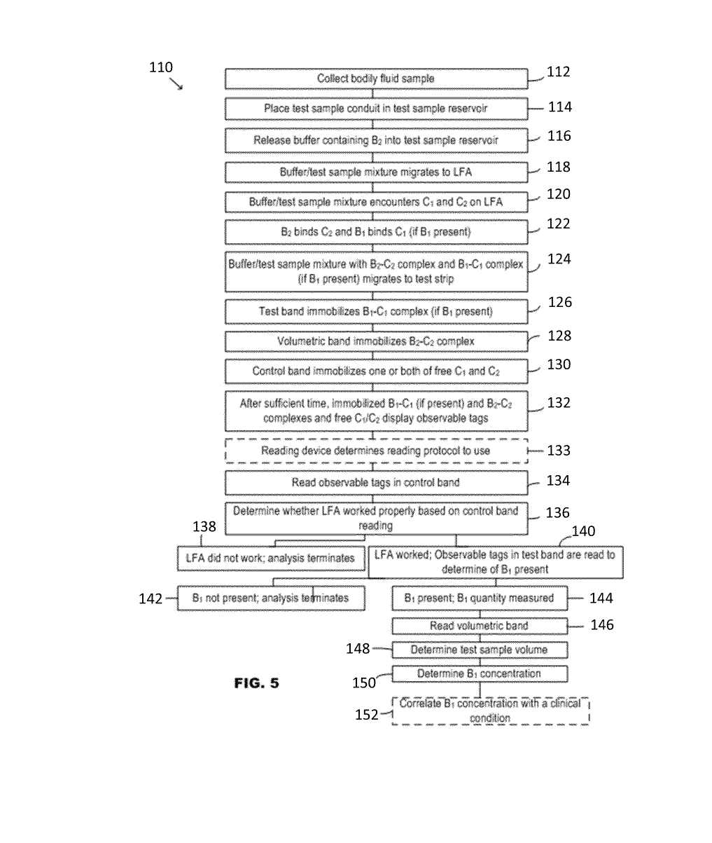

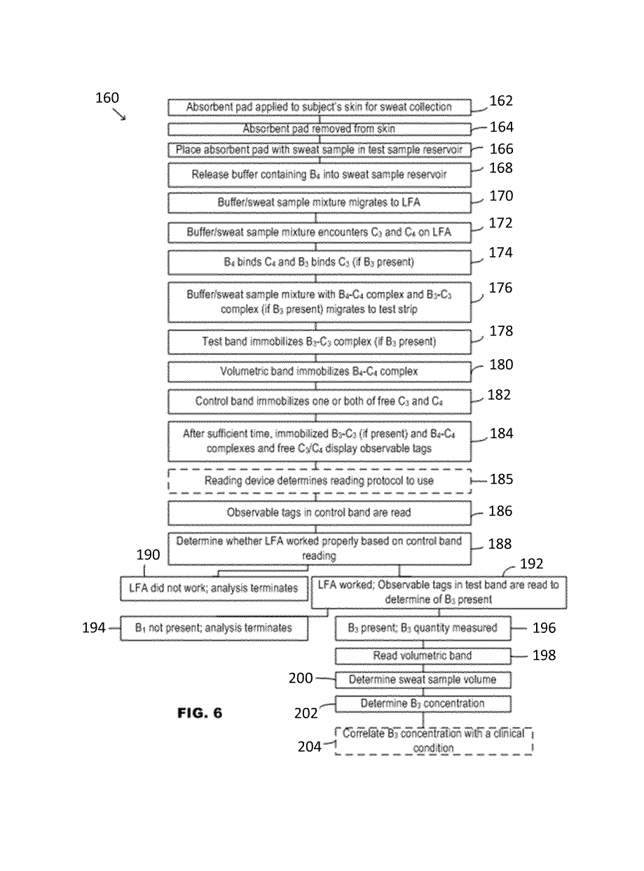

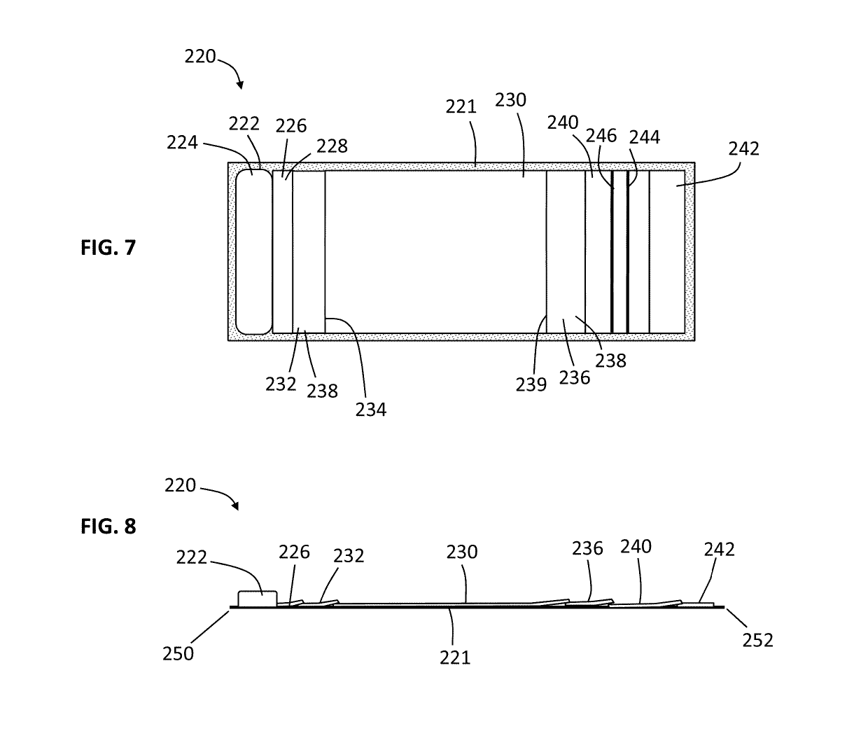

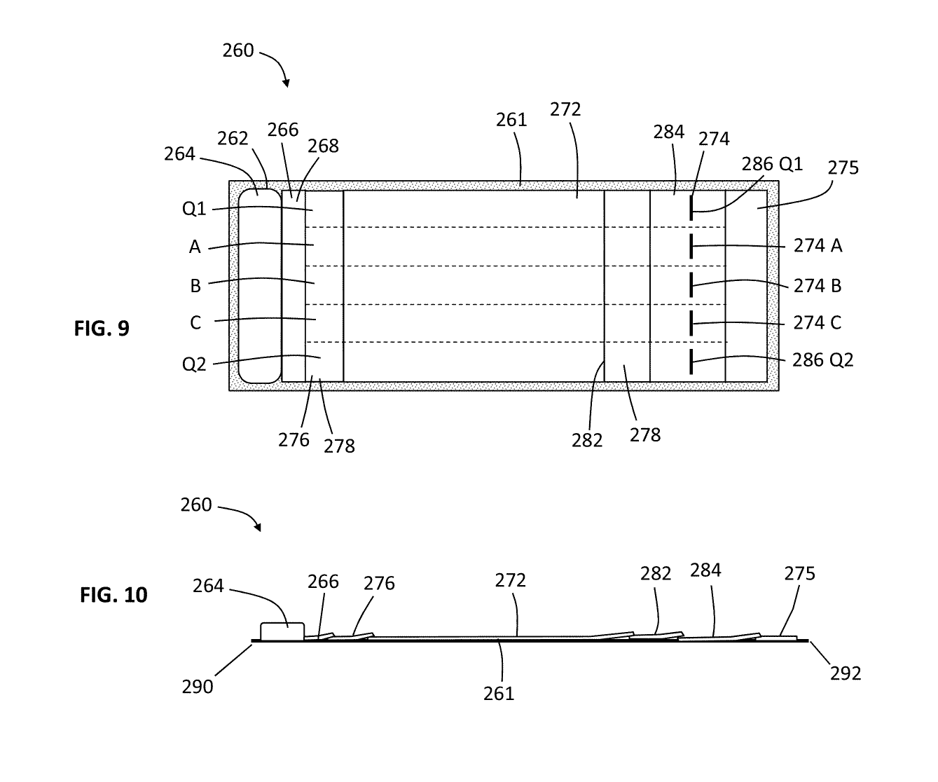

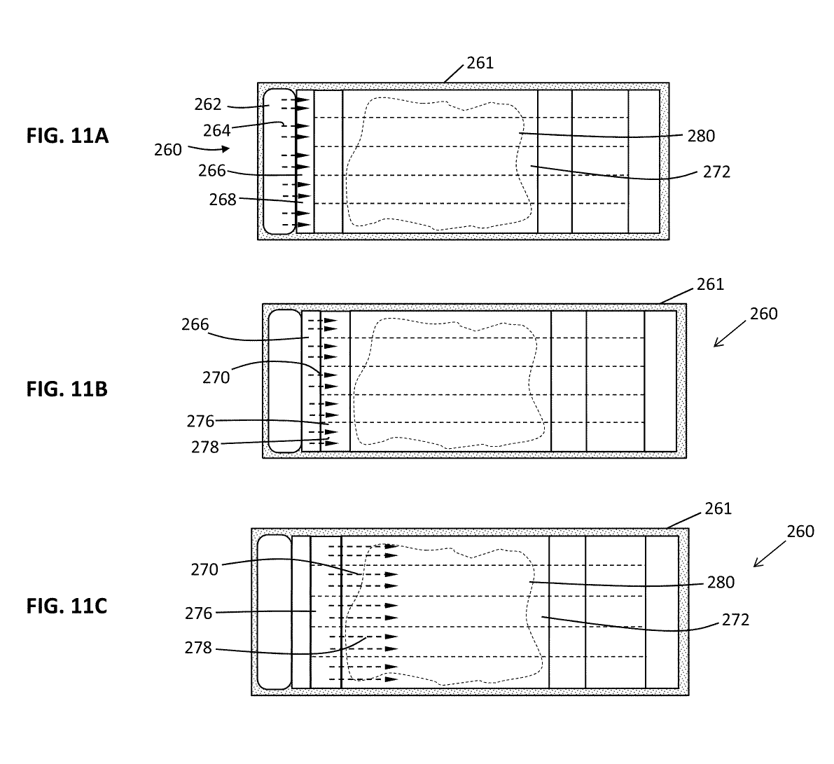

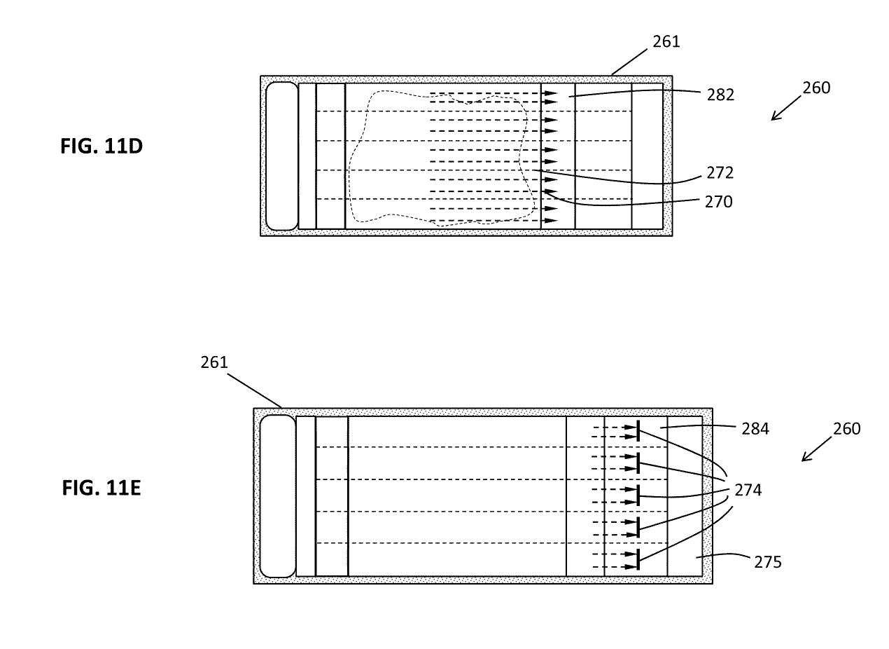

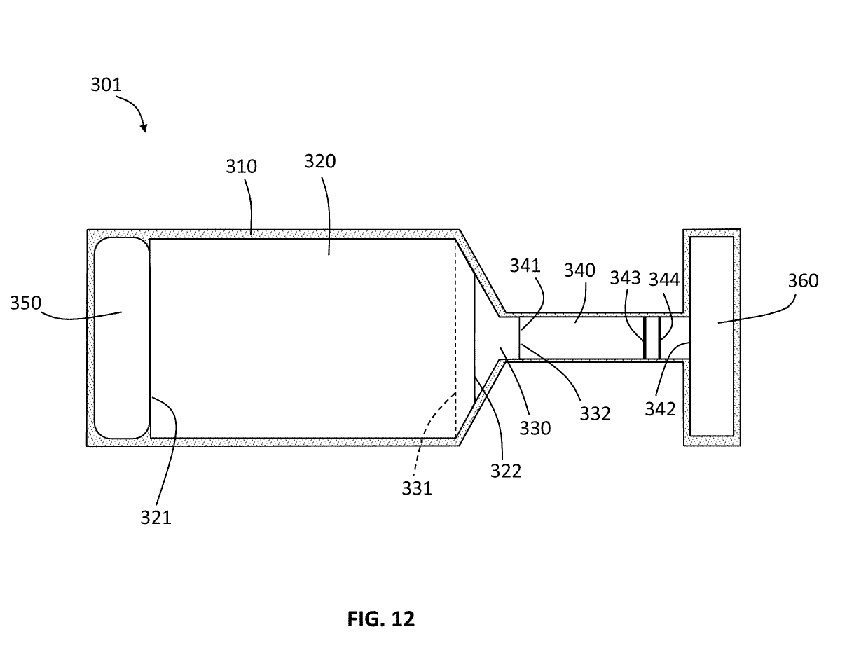

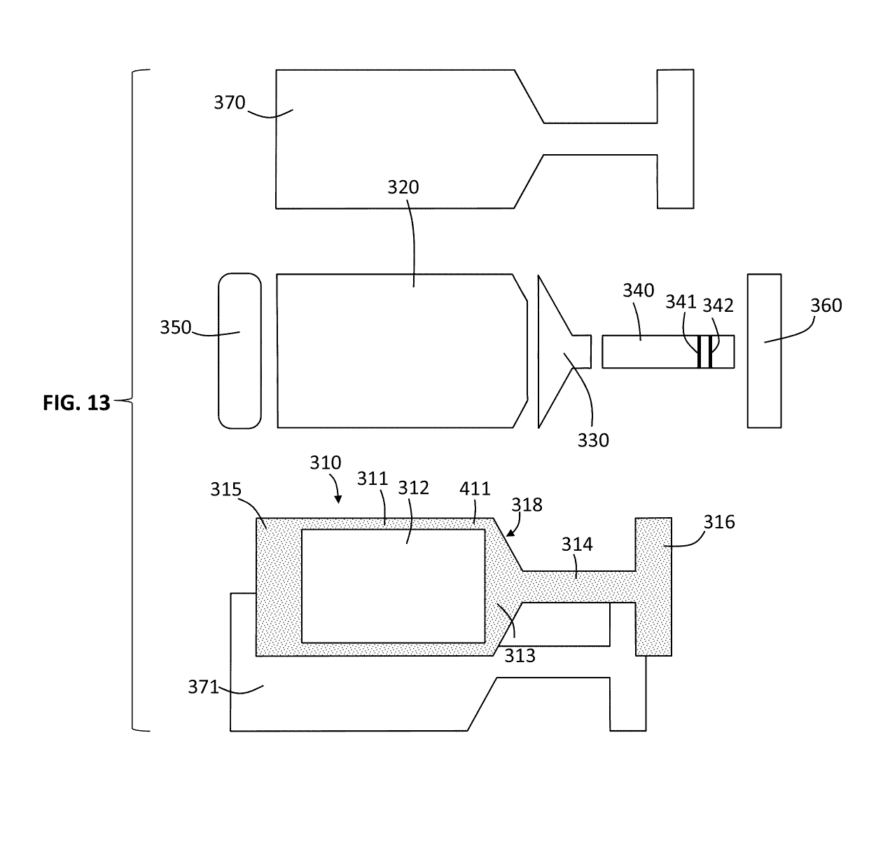

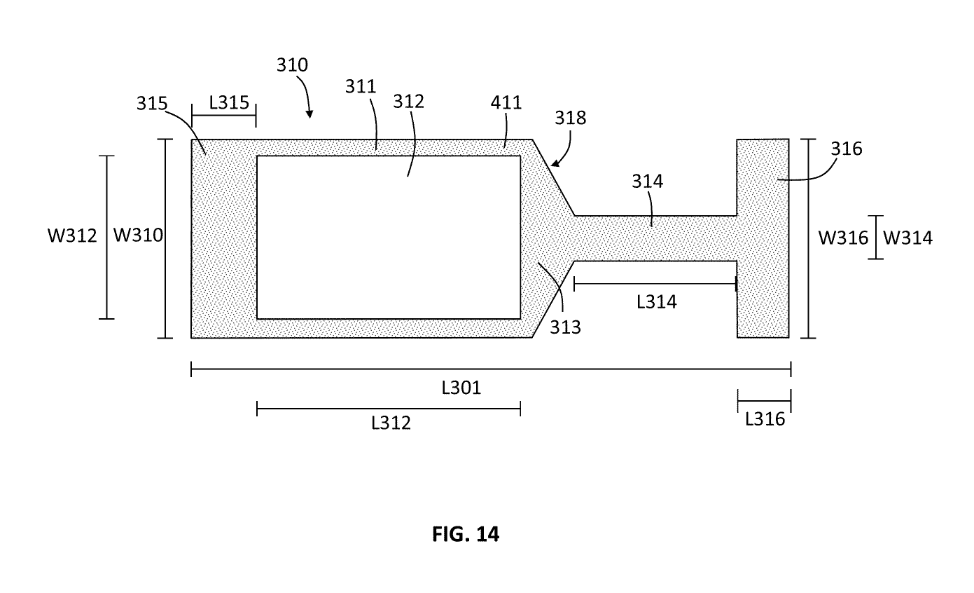

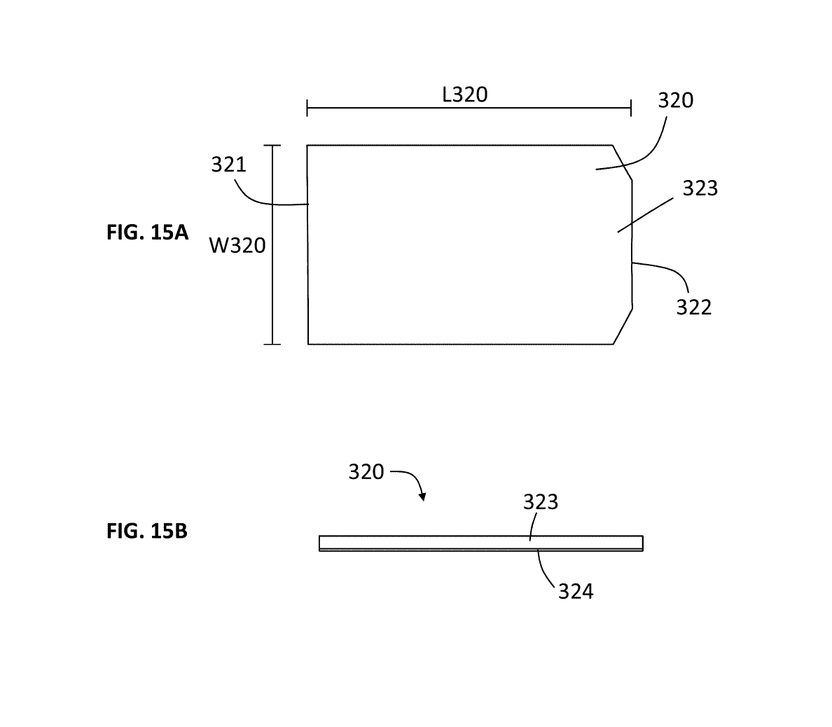

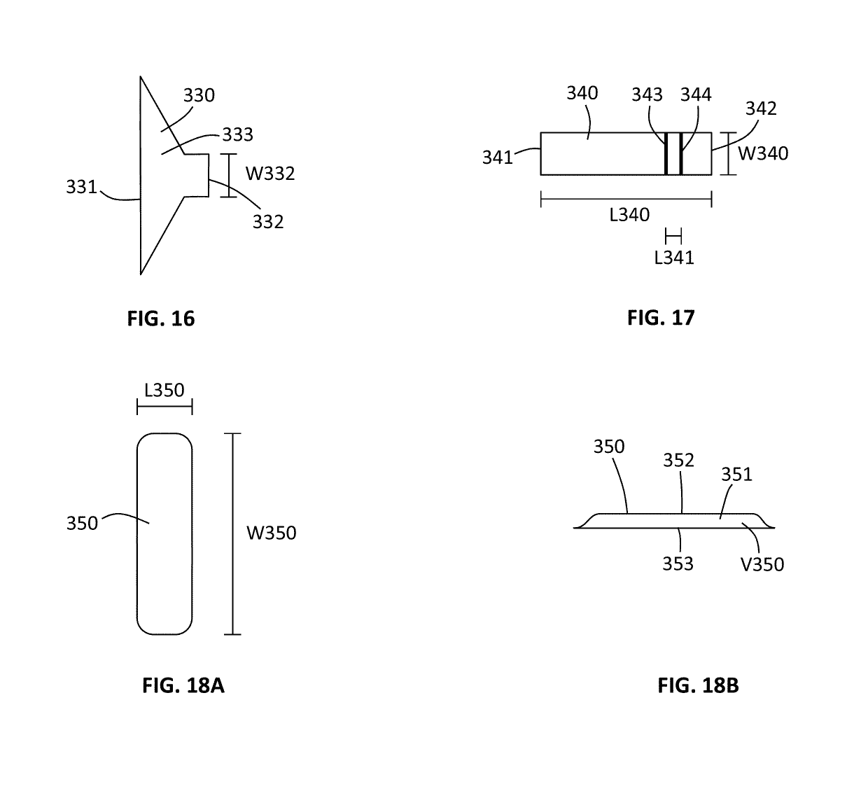

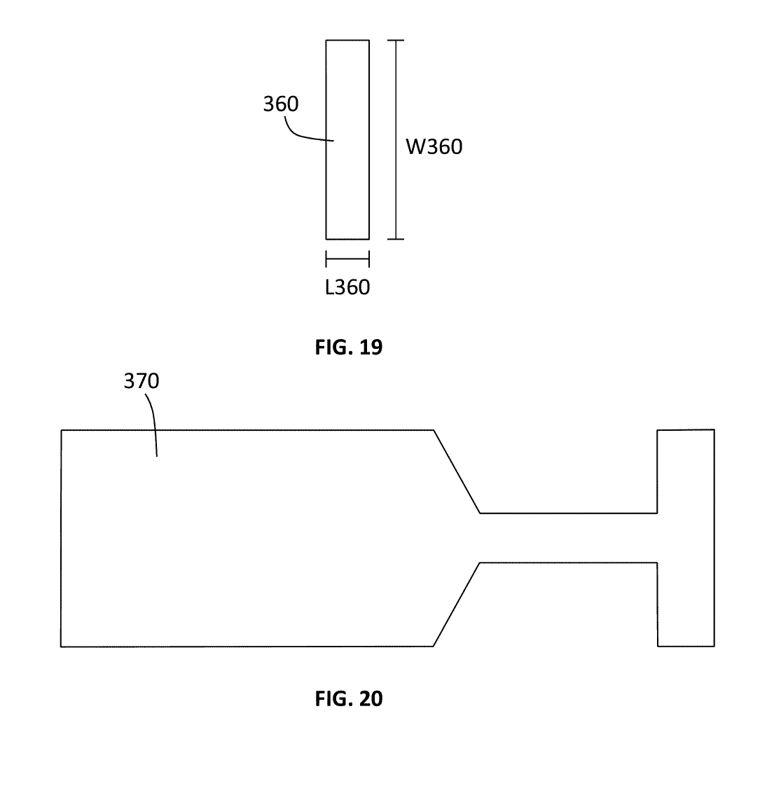

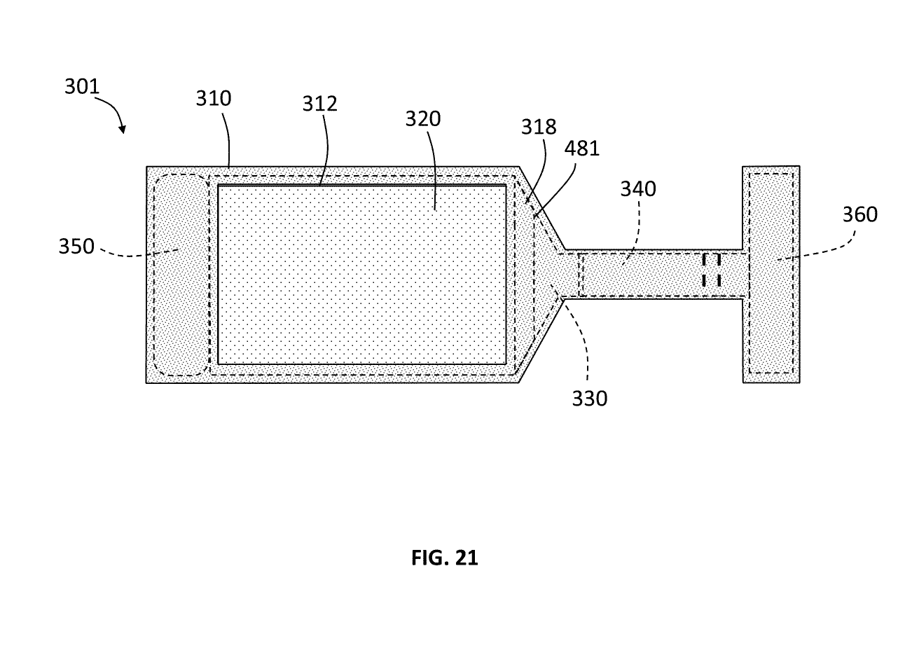

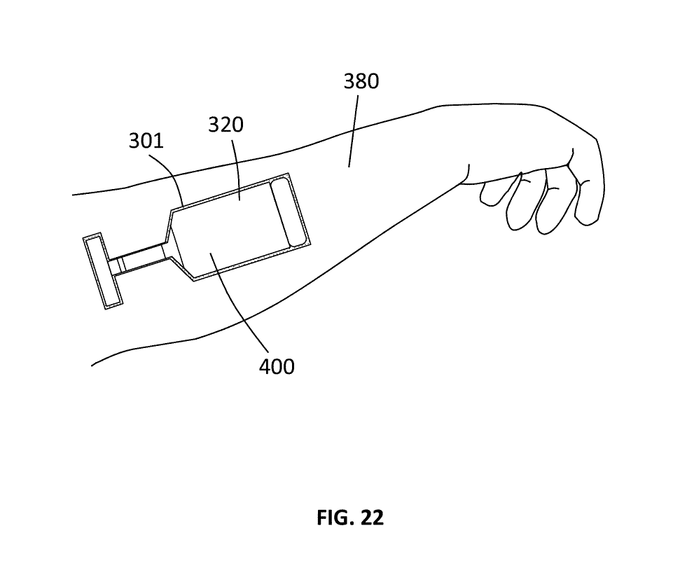

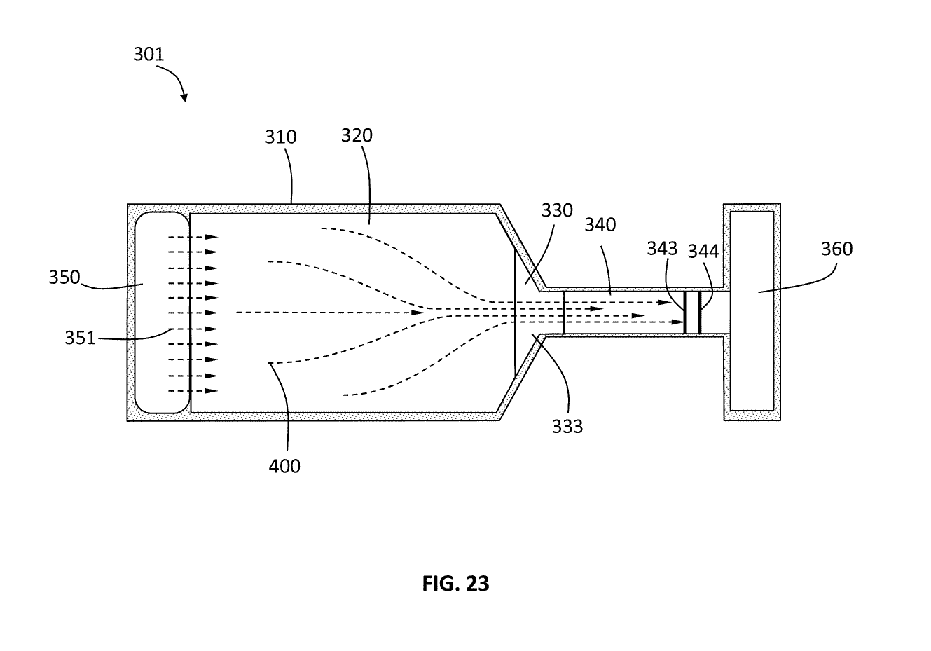

FIG. 2 is a bottom view of an embodiment of a test sample element 48. FIG. 3 is an expanded, schematic, side, cross-sectional view of the test sample element of FIG. 2. As shown in FIGS. 2 and 3, the test sample element includes a top 50 and a bottom 52. In this example, the test sample element comprises an absorption component 54. In some embodiments, the test sample element also includes one or more of an adhesive component 56, a release component 58, an identification component 60, and/or a contact membrane 62, each of which may be optionally included in the test sample element. Although each of these components are shown in FIG. 2, it should be appreciated that not all components need be present in some embodiments. For instance, in some cases the test sample element does not include one or more of the adhesive component, release component, identification component, and/or contact membrane. For example, in some embodiments in which a contact membrane is not present, the absorption component may be an outermost layer of the test sample element. For instance, the skin of a patient/user may be in direct contact with the absorption component during use. In other embodiments, an adhesive component may be an outermost layer of the test sample element. Other configurations are also possible.

In some embodiments, one or more of the components that make up the test sample element are hypoallergenic.

The absorption component may include an absorbent material that is configured to absorb a sample (e.g., a bodily fluid sample such as sweat, blood, urine, lavage, plasma, saliva, tears). The absorption component may have any suitable shape, e.g., a rectangle, a modified rectangle with tapered corners, or a circle. In some embodiments, the absorption component is sized and shaped to fit within a region (e.g., region 18 shown illustratively in FIGS. 1B and 1C) of an analysis device. In some embodiments, the absorption component has an absorbent, wicking, and/or capillary property that will not chemically react with a liquid (e.g., buffered solution, water) contained in the device or sample (e.g., bodily fluid sample) that the absorption component is exposed to.

In some embodiments, such as when the bodily fluid is a bodily fluid collected from a person's skin (e.g., sweat), the absorption component is a fluid collection pad that includes a low-binding, hydrophilic material that is pliable and capable of forming to the contours of the skin (e.g., on an arm) of the subject. The absorption component may be formed of any suitable material such as non-synthetic/natural polymers (e.g., cellulose, high density cellulose, regenerated cellulose, cellulose acetate, cotton, wood pulp, hemp), synthetic polymers, glass fibers, and other natural or artificial absorbent materials. Specific examples of such materials include, but are not limited to, woven or nonwoven materials of natural or synthetic fibers, such as rayon, polyester, polyurethane, polyolefin, cellulose, cellulose derivatives, cotton, orlon, nylon, hydrogel polymeric material; glass fibers, and combinations thereof. For example, in some embodiments, FUSION 5™ available from GE Healthcare in Fairfield, Conn. may be used. FUSION 5™ is a single layer matrix hydrophilic material, and is also described as a glass fiber-based membrane treated with a plastic binder having appropriate hydrophilic properties.

In some embodiments, the absorption component is textured to enhance its absorption characteristics and/or to reduce the possibility of leakage of the sample following absorption but prior to elution by a buffered solution. The absorption component may be applied directly to the subject's skin to collect fluid (e.g. sweat) therefrom. Alternatively, in some embodiments, the absorption component includes an additional contact membrane (e.g. contact membrane 62) that separates the absorption component from the subject's skin. The contact membrane 62 may reduce skin irritation and/or prevent loss of the collected fluid sample or analyte from the absorption component back onto the skin. The contact membrane may be permeable and may optionally be permeable in one direction only, allowing absorption of bodily fluid into the absorption component, but preventing flow from the absorption component back onto the skin. In one example, the contact membrane comprises a discontinuous (e.g., porous) polyolefin layer.

In examples in which the test sample element is intended to collect bodily fluid from a subject's skin, the bottom of the test sample element may be placed on the skin. In some of these examples, the adhesive component (e.g., adhesive component 56) is a film that at least partially circumscribes the absorption component (e.g., absorption component 54) and is configured to adhere the test sample element to the subject's skin for at least a minimum threshold length of time. In some embodiments, the adhesive component also covers the absorption component. For example, the adhesive component may be configured to hold to the skin for a minimum period of minutes, such as five minutes or more. Alternatively, the adhesive component may be configured to hold to the skin for minimum period of days, such as seven days or more.

In some embodiments, particularly when the bodily fluid being collected is sweat, the adhesive component and/or the absorption component may be treated or otherwise configured to promote sweat secretion from the subject's skin. To collect sweat, the test sample element may be placed on the subject's skin for a period of time sufficient to collect a threshold volume of sweat for a concentration analysis performed by the analysis device. In some embodiments, the amount of sweat collected is in a range from about 1 μL to about 500 μL, although more or less sweat than this may be collected to test for a target analyte using an analysis device described herein.

In other embodiments, a test sample element (e.g., test sample element 48) is adapted and arranged to collect other samples (e.g., bodily fluids samples such as blood, urine, lavage, plasma, saliva, tears).

In some embodiments, a test sample element may have a particular volume capacity for collecting a specified volume (or range of volume) of sample (e.g., bodily fluid sample). In some embodiments, the test sample element has a volume capacity of at least 1 μL, at least 10 μL, at least 20 μL, at least 50 μL, at least 75 μL, at least 100 μL, at least 125 μL, at least 150 μL, at least 175 μL, at least 200 μL, at least 300 μL, or at least 400 μL. In certain embodiments, the test sample element has a volume capacity of less than or equal to 500 μL, less than or equal to 400 μL, less than or equal to 300 μL, less than or equal to 200 μL, less than or equal to 100 μL, less than or equal to 75 μL, less than or equal to 50 μL, less than or equal to 25 μL, or less than or equal to 10 μL of sample can be used. Combinations of the above-referenced ranges are also possible (e.g., at least 10 μL and less than or equal to 125 μL). Other values and ranges are also possible. The volume capacity of test sample element may be greater than or equal to the volume of sample collected. In some cases, the volume capacity of the test sample element also accounts for the volume of any liquid (e.g., buffered solution, water) to be used in the analysis.

Accordingly, any suitable amount of sample can be collected and/or introduced into a device described herein (e.g., using a test sample element). In some embodiments, at least 1 μL, at least 10 μL, at least 20 μL, at least 50 μL, at least 75 μL, at least 100 μL, at least 125 μL, at least 150 μL, or at least 175 μL of sample can be used. In certain embodiments, less than or equal to 500 μL, less than or equal to 400 μL, less than or equal to 300 μL, less than or equal to 200 μL, less than or equal to 100 μL, less than or equal to 75 μL, less than or equal to 50 μL, less than or equal to 25 μL, or less than or equal to 10 μL of sample can be used. Combinations of the above-referenced ranges are also possible (e.g., at least 10 μL and less than or equal to 125 μL). Other values and ranges are also possible.

In some embodiments, one or more portions of the test sample element includes one or more tamper evident features. For example, an adhesive component may include one or more perforations that tear when removed from the subject's skin. Thus, if the person administering the test sample element to the skin finds, prior to removal of the test sample element from the skin, that a perforation has been torn, it may be assumed that the test sample element has been tampered with, and that appropriate measures (e.g., re-administration of the test sample element and/or punitive measures) be carried out.

In some embodiments in which an adhesive component (e.g., adhesive component 56) covers an absorption component (e.g., absorption component 54), a release component (e.g., release component 58) may be disposed between the absorption component and the adhesive component. The release component may be configured to facilitate detachment of the absorption component from the adhesive component. In some embodiments the release component is a non-adhesive film, membrane, or webbing that adheres to the adhesive component but does not substantially adhere to the absorption component. Upon detachment of the absorption component from the adhesive component, the absorption component may be placed in the appropriate region (e.g., region 18 of the analysis device shown in FIGS. 1B and 1C) and the lateral flow assay may then be initiated.

The test sample element may be adapted and arranged to be used once and then disposed, e.g., along with the analysis device, after the analysis has been completed.

In embodiments in which the test sample element includes an identification component (e.g., identification component 60), the identification component may include information (e.g., in the form of text, a QR code, and/or a RFID tag) about the test sample element. In some embodiments, the information provided by the identification component may include one or more of: which bodily fluid the test sample element is intended for; the date(s) of manufacture and/or packaging of the test sample element; a source identifying logo; and/or a random serial number. If the test sample element is configured to collect sweat from skin, the identification component may also contain information regarding how long the test sample element is designed to be placed on the skin for adequate sweat collection.

In some particular embodiments, and as shown illustratively in FIGS. 2 and 3, the test sample element, the absorption component 54, and the adhesive component 56 are round, the absorption component having a diameter d₁ and the adhesive component having a diameter d₂. In some embodiments, d₁ is in a range from about 0.5 cm to about 5 cm, and d₂ is larger than d₁ and is in a range from about 1 cm to about 20 cm. In a particular example, d₁ is about 2 cm and d₂ is about 5 cm. Dimensions outside of these values and ranges would also be suitable.

The test sample element and/or absorption component may have any suitable thickness. In some cases, the test sample element and/or absorption component may have a thickness of at least 0.1 mm, at least 1.0 mm, at least 2.0 mm, at least 5.0 mm, at least 10.0 mm, or at least 20.0 mm. In some instances, the test sample element and/or absorption component has a thickness of less than or equal to 30.0 mm, less than or equal to 20.0 mm, less than or equal to 10.0 mm, or less than or equal to 5.0 mm. Combinations of the above-referenced ranges are also possible (e.g., at least 1.0 mm and less than or equal to 10.0 mm). Other ranges are also possible. The thickness of absorption component 54 of test sample element 48 is shown as thickness t₁ in FIG. 3.

FIG. 4 is a top schematic view of an embodiment of an analysis device (e.g., a bodily fluid analysis device) 70 for analyzing a sweat sample. Although the following description will assume analysis of a sweat sample, it should be appreciated that the device 70 can alternatively be used to analyze analytes in other bodily fluids as described herein.

As shown illustratively in FIG. 4, analysis device 70 includes a device body 72, a first end 74, a second end 75, a liquid reservoir 76, a buffer 78 initially contained within the liquid reservoir, a region 80 for containing a test sample element and/or sample, a test sample element 82, a lateral flow assay 84 including a conjugate region 86, a test strip 88, a results window 90, a test band 92, a volumetric band 94, a control band 96, and an optional wicking element 98. The analysis device also includes an optional lid 100 and an optional device identifying component 102. In some cases, lid 100, if present, may be hingedly attached along one edge to the device body 72. The device body 72 houses the liquid reservoir 76, the buffer 78, an optional liquid releasing member (not shown), the region 80 for containing a test sample element and/or sample, the test sample element 82, the lateral flow assay 84, the device identifying component 102 and, when closed, the lid 100. As shown illustratively in this figure, the device body includes a first region 104, a second region 106, and a third region 108.

It should be appreciated that while FIG. 4 shows certain components of a device, not all components need be present in certain embodiments. For example, in some cases the device does not include a wicking element. It should also be understood that the configuration of positioning of components shown in the figure may vary. Additionally, other components not shown in the figure may be present in certain embodiments.

In one version of analysis device 70, the test sample element 82 may be applied to the subject's skin for a period of time to collect at least a threshold amount of sweat. In this example, the subject's sweat is being tested for the presence and concentration of a target analyte B₁. The test sample element 82 is then removed from the subject's skin and placed in the sweat sample reservoir 80. Buffer 78 may be contained in a liquid reservoir (e.g., a blister pack) prior to use of the device (e.g., prior to introduction of a sample into the device). As described herein, the liquid reservoir may be associated with an optional liquid releasing member (not shown), In other embodiments, the buffer may be contained in a liquid reservoir (e.g., blister pack) that can be removably positioned in the device (e.g., by a user). The buffer may contain a volumetric analyte B₂ of known concentration. The volumetric analyte B₂ is different from the target analyte B₁ and is known not to chemically react with sweat or with any portion or portions of the analysis device. Once the test sample element has been placed in region 80 (and/or the buffer has been placed in the device), the lid 100 is closed. In FIG. 4, for ease of reference to the features underneath the lid 100, the lid 100 is represented as being transparent and in the closed position.

As shown illustratively in FIG. 4, the first region 104 of the device body 72 houses the liquid reservoir 76, the buffer 78 (initially), the sweat sample reservoir 80, and the test sample element 82. The second region 106 of the device body 72 houses the lateral flow assay 84. The third region 108 of the device body 72 houses the device identifying component 102. It should be appreciated that the device need not include three separate regions, and that other configurations of the device are also possible (e.g., one region, two regions, four regions, etc.), with different combinations of components in each region.

In some embodiments, the components of the analysis device are positioned within the device body such that the liquid reservoir 76 is in fluid communication with (e.g., in adjacent contact with or overlaps with) the test sample element 82 (e.g., during use), the eluted sweat sample is in fluid communication with conjugate region 86, the conjugate region is in fluid communication with the test strip 88, and the test strip is in fluid communication with the optional wicking element 98.

In certain embodiments, closure of the lid 100 releases the buffer 78 and may also irreversibly lock the analysis device as described herein. For instance, closure of the lid may cause a liquid releasing member to puncture or break a portion of the liquid reservoir to allow release of the liquid contained therein. The released buffer 78 flows in the direction A₂ into region 80 where it mixes with the sweat absorbed in the test sample element 82, eluting the target analyte B₁ (to the extent it is present) in the direction A₂ and onto the lateral flow assay 84. Once contacting the lateral flow assay 84, the sweat/buffer mixture may be wicked in the direction A₂ via capillary action provided or enhanced by the wicking element 98, which is optionally disposed towards the second end 75 of the analysis device. The wicking element can be used to draw the sweat/buffer mixture along the lateral flow assay 84 towards the second end 75. As the sweat/buffer mixture migrates along the direction A₂, it contacts the conjugate region 86, where the volumetric analyte B₂ binds with a corresponding volumetric conjugate positioned (e.g., previously deposited) in the conjugate region 86, the volumetric conjugate having an observable tag. Also in the conjugate region 86, if the target analyte B₁ is present in the sweat sample, the target analyte B₁ binds with a corresponding target analyte conjugate having an observable tag, the target analyte conjugate also positioned (e.g., having been previously deposited) in the conjugate region 86. Though a single conjugate region (e.g., containing two different conjugates) is shown in the device, in other embodiments separate conjugates can be positioned in separate locations of the conjugate region, or in different conjugate regions.

The volumetric analyte-conjugate complex and, if present, the target analyte-conjugate complex, along with the remaining sweat buffer mixture then migrate to the test strip 88, where they encounter the volumetric band 94. The volumetric band may include particles or other entities that bind the volumetric analyte conjugate complex, the particles or other entities having been previously deposited and immobilized in the test strip. The volumetric band may be adapted and arranged to immobilize the volumetric analyte conjugate complex proportionally to the volumetric analyte's concentration in the sweat/buffer mixture. The visual tags on the immobilized volumetric molecules appear in the form of volumetric band 94 (e.g., through a color or any other observable change).

The remaining sweat/buffer mixture continues to migrate in the direction A₂ and encounters the test band 92. The test band may include particles or other entities that bind the target analyte conjugate complex, the particles or other entities having been previously deposited and immobilized in the test strip. If any target analyte conjugate complexes are present in the fluid mixture, the test band immobilizes such molecules, and the observable tags on the immobilized target analyte conjugate appear in the test band (e.g., through a color or any other observable change).

The remaining sweat/buffer mixture continues to migrate in the direction A₂ and encounters the control band 96. The control band may include particles or other entities that bind the volumetric analyte conjugate and/or the target analyte conjugate, the particles or other entities having been previously deposited and immobilized in the test strip. If the lateral flow assay worked properly, the control band may immobilize all or a portion of the remaining volumetric conjugate and/or the target analyte conjugate, and the visual tags on the immobilized conjugate(s) appear in the control band. Appearance of the control band 96 (e.g. through a color or any other observable change) beyond a threshold level of observable intensity confirms that sufficient migration of the sweat/buffer mixture through the lateral flow assay occurred. The test band 92 (if showing), the volumetric band 94, and the control band 96 may be observable and/or determined (e.g., quantitatively) through the results window 90.

A programmable reader (e.g., an optical reader) may be used to read the device identifying component 102 (e.g., a passive RFID, a barcode, a QR code, a bluetooth-enabled component, or a Wi-Fi-enabled component) and/or to run a script (e.g., a series of instructions) based on the information provided by the device identifying component. The optical reader may be programmed to perform readings of a plurality of different analyses. For example, the optical reader may be programmed to run different scripts depending on the target analyte being tested, the volumetric analyte being tested, and/or the bodily fluid being tested. The information provided to the optical reader by the device identifying component 102 determines which script (e.g., which analysis to run) when reading the results of the lateral flow assay. Once the appropriate script for the optical reader has been selected, the optical reader is aligned with the results window 90. In a particular example, the optical reader is the OPTRICON CUBE-READER™ (opTricon GmbH, Berlin, Germany).

The optical reader may be programmed to distinguish between the test band 92, the volumetric band 94, and the control band 96. The optical reader may collect data from each band's intensity, the intensity correlating with the quantity or number of immobilized conjugate molecules in each band. The optical reader processes the data collected from reading each of the bands. For example, the optical reader may processes information regarding the intensity of the control band to determine whether the assay performed properly; the optical reader may process information regarding the intensity of the test band to determine a quantity of the target analyte B₁ present in the tested sample (e.g., sweat sample); and the optical reader may process information regarding the intensity of the volumetric band to determine a volume of the tested sample.

The optical reader may be programmed, based on the data, to display one or more results. For example, the optical reader may display: a) whether the assay performed properly; b) if the assay performed properly, whether or not the target analyte B₁ was present in the sample; c) if the target analyte B₁ was present, its quantity in the sample; d) the volume of the sample; and/or e) the concentration of the target analyte B₁ in the sample based on a quotient of the target analyte quantity and the sample volume. One or more of these results may then be associated with a clinical condition, e.g., a level of impairment.

In the example analysis device 70, the device body 72 has a width w₁. The first region 104 of the device body 72 has a length l₁. The second region 106 of the device body 72 has a length l₂ and a width w₂. The third region 108 of the device body 72 has a length l₃. In some embodiments, l₁ is in a range from about 20 mm to about 60 mm; l₂ is in a range from about 20 mm to about 60 mm; l₃ is a range from about 5 mm to about 30 mm; w₁ is in a range from about from about 20 mm to about 60 mm; and w₂ is in a range from about 1 to about 20 mm. In a particular example, l₁ is about 40 mm, l₂ is about 41 mm, l₃ is about 20 mm, w₁ is about 40 mm and w₂ is about 21. It should be appreciated that other dimensions are also possible.

The first region 104 and the third region 108 of the device body 72 may have a thickness (into the page in FIG. 4) t₂. In some embodiments, t₂ is in a range from about 3 mm to about 20 mm. In a specific example, t₂ is about 10 mm. In some embodiments, the top of the second region 106 of the device body 72 is depressed (into the page in FIG. 4) relative to the tops of the first region 104 and the third region 108 of the device body 72. In these examples, the second region 106 of the device body 72 has a thickness t₃ (into the page in FIG. 4) that is less than t₂. In some embodiments, t₃ is in a range from about 1 mm to about 10 mm. In a particular example, t₃ is about 5 mm. It should be appreciated that other dimensions are also possible.

In certain embodiments, the lid 100 is shaped and sized to cover the liquid reservoir 76 and region 80 which may contain the test sample element. In this example, the lid 100 is shaped and sized to be housed by the device body 72 when the lid 100 is closed.

In some embodiments, one or more of the dimensions of the device (e.g., dimensions l₂, w₂, and t₃) may be selected, at least in part, to complement features and/or dimensions of a particular optical reader and thereby facilitate reading of the lateral flow assay by an optical reader. For example, the OPTRICON CUBE-READER™ is approximately cube shaped with a recessed strip on one of the cube's six faces. Optical reading of a test strip can be performed when a lateral flow assay is placed within the recessed strip. Thus, in at least one example, the dimension l₂, w₂, and t₃ are selected so that the lateral flow assay 84 and the second region 106 of the device body 72 that houses the lateral flow assay 84 can be inserted in the recessed strip of the OPTRICON CUBE-READER™. It should be appreciated, however, that other optical readers and detection systems can be used.

In some embodiments, at least the collection of the sweat sample, release of the buffer and completion of the lateral flow assay occurs while an analysis device described herein (e.g., analysis device 70) is positioned on the skin of the subject. In some of these examples, the analysis device is secured to the skin by any suitable means for a period of time at least long enough to: a) collect a sufficiently large sweat sample for analysis; and b) to perform a lateral flow assay with the sweat sample to completion. In some of these examples, the test sample element is at least initially exposed to the surface of the subject's skin in order to absorb sweat therefrom, while the results window faces away from the skin in order to allow reading thereof.

FIG. 5 is a flow chart illustrating an embodiment of a method analysis 110 for determining the concentration of a target analyte in a bodily fluid. It should be understood that each step need not be present in certain methods described herein. Additionally, in some embodiments the order of the steps may be different than that shown in FIG. 5.

In step 112, a sample of bodily fluid being tested for a target analyte B₁ is collected on a test sample element. In step 114, the test sample element containing the sample (e.g., bodily fluid sample) is placed in a region of an analysis device such as a test reservoir. It should be appreciated, however, that in some embodiments steps 112 and 114 may be combined (e.g., in embodiments in which the test sample element is integrally attached to a body portion of the device during sample collection). In step 116, a buffered solution containing a known concentration of a volumetric analyte B₂ is released into the region containing the sample. In step 118, a mixture of the buffered solution and test sample migrates to a lateral flow assay (“LFA”) contained within the analysis device.

In step 120, the mixture of buffered solution and test sample encounters a conjugate region on the lateral flow assay on which conjugates C₁ and C₂ have been disposed. Each of C₁ and C₂ may include a tag observable on a lateral flow assay test strip. C₁ is specific to target analyte B₁ and known to bind B₁. C₂ is specific to volumetric analyte B₂ and known to bind B₂. In step 122 B₂ binds C₂ and, if B₁ is present in the test sample, B₁ binds C₁. In step 124, the mixture of buffer and solution and test sample containing the B₂-C₂ complex and, if B₁ is present in the test sample also the B₁-C₁ complex, migrates to a test strip portion of the lateral flow assay. In step 126 a test band on the test strip immobilizes the B₁-C₁ complex present in the mixture. In step 128, a volumetric band on the test strip immobilizes the B₂-C₂ complex. In step 130, a control band immobilizes one or both of free C₁ and C₂ molecules. In step 132, sufficient time passes for immobilized B₁-C₁ molecules in the test band, immobilized B₂-C₂ molecules in the volumetric band, and immobilized free C₁ and/or C₂ molecules in the control band to display their observable tags.

In an optional step 133, a reading device (e.g., detector) determines which reading protocol to perform on the analysis device based on the reading device detecting the identity of the analysis device. In step 134, the observable tags in the control band are read. In step 136, based on the reading of the observable tags in the control band a determination is made as to whether the lateral flow assay operated properly. In step 138, if the lateral flow assay did not work properly, the analysis terminates. In step 140, if the lateral flow assay did work properly, the observable tags in the test band are read to determine whether the target analyte B₁ was present in the sample. In step 142, if B₁ was not present, the analysis terminates. In step 144, if B₁ was present, the quantity of B₁ is measured from the observable tags in the test band. In step 146, the volumetric band is read. In step 148, based on the reading of the observable tags in the volumetric band, a determination is made as to the volume of the sample (e.g., bodily fluid sample) that was tested. In step 150, a calculation of the concentration of B₁ in the sample (e.g., bodily fluid sample) is performed based on the measured quantity of B₁ and the measured volume of the sample. In an optional step 152, the concentration of B₁ in the sample is correlated with a clinical condition of the test subject from whom the sample was collected.

FIG. 6 is a flow chart illustrating an embodiment of a method analysis 160 for determining the concentration of a target analyte B₃ in sweat. In step 162, a test sample element such as an absorbent pad is applied to a subject's skin for a period of time sufficient to collect a threshold volume of sweat to perform the analysis. In step 164 the absorbent pad is removed from the skin. In step 166 the absorbent pad containing the sweat sample is introduced into an analysis device (e.g., placed in a sweat sample reservoir in an analysis device). Step 166 can occur at any appropriate time following completion of step 164. For example, step 166 may be started immediately or after a period of delay, depending on the intended use and/or the target analyte. In some embodiments, step 166 occurs within about five minutes, about ten minutes, about thirty minutes, about one hour, about four hours, about twenty-four hours, about one week, or about two weeks after step 164.

In step 168, a buffered solution known not to react chemically with sweat and containing a known concentration of a volumetric analyte B₄ is released into the sweat sample reservoir. In step 170, a mixture of the buffered solution and sweat sample migrates to a lateral flow assay contained within the analysis device. In some embodiments, migration of the buffered solution and/or sweat sample is facilitated by a wicking element, and the buffered solution and/or sweat sample migrates towards the wicking element. In step 172, the mixture of buffered solution and sweat sample encounters a conjugate region on the lateral flow assay on which conjugates C₃ and C₄ have been disposed. Each of C₃ and C₄ includes a tag observable on a lateral flow assay test strip. C₃ is specific to target analyte B₃ and known to bind B₃. C₄ is specific to volumetric analyte B₄ and known to bind B₄. In step 174 B₄ binds C₄ and, if B₃ is present in the sweat sample, B₃ binds C₃. In step 176, the mixture of buffer and solution and sweat sample containing the B₄-C₄ complex and, if B₃ is present in the sweat sample also the B₃-C₃ complex, migrates to a test strip portion of the lateral flow assay. In step 178 a test band on the test strip immobilizes the B₃-C₃ complex present in the mixture. In step 180, a volumetric band on the test strip immobilizes the B₄-C₄ complex. In step 182, a control band immobilizes one or both of free C₃ and C₄ molecules. In step 184, sufficient time passes for immobilized B₃-C₃ molecules in the test band, immobilized B₄-C₄ molecules in the volumetric band, and immobilized free C₃ and/or C₄ molecules in the control band to display their observable tags.

In an optional step 185, a reading device (e.g., detector) determines which reading protocol to perform on the analysis device based on the reading device detecting the identity of the analysis device. In step 186, the observable tags in the control band are read. In step 188, based on the reading of the observable tags in the control band a determination is made as to whether the lateral flow assay operated properly. In step 190, if the lateral flow assay did not work properly, the analysis terminates. In step 192, if the lateral flow assay did work properly, the observable tags in the test band are read to determine whether the target analyte B₃ was present in the sweat sample. In step 194, if B₃ was not present, the analysis terminates. In step 196, if B₃ was present, the quantity of B₃ is measured from the observable tags in the test band. In step 198, the volumetric band is read. In step 200, based on the reading of the observable tags in the volumetric band, a determination is made as to the volume of the sweat sample that was tested. In step 202, a calculation of the concentration of B₃ in the sample is performed based on the measured quantity of B₃ and the measured volume of the tested sweat sample. In an optional step 204, the concentration of B₃ in the sweat sample is correlated with a clinical condition of the test subject from whom the sweat sample was collected.

FIG. 7 is a top schematic view of a further embodiment of an analysis device (e.g., a bodily fluid analysis device) 220. FIG. 8 is a side schematic cross-sectional view of the analysis device 220 of FIG. 7. As shown illustratively in FIGS. 7 and 8, the analysis device has a device body 221 comprising a two-part liquid reservoir, including a liquid reservoir 222 that holds a liquid 224 (e.g., water or other suitable liquid such as a buffer) and a buffer pad 226 with buffer components 228 (e.g., one or more salts, sugars, proteins, surfactants, polymers, etc.) deposited onto it. In some embodiments, the buffer components include a non-target analyte such as a volumetric analyte (e.g., biotin) of known quantity as described above. The liquid reservoir 222 may have a breakable seal (e.g., similar to a blister pack) as described herein, so that the liquid may be released from it, e.g., after the sample (e.g., bodily fluid sample) has been collected onto the test sample element 230 and is ready for analysis.

The buffer pad may be constructed of any suitable material, such as glass fiber, cellulose, rayon, other fibrous materials, or combinations thereof. In some embodiments, the buffer pad has the same characteristics of a conjugate region (e.g., conjugate pad) described herein. When the liquid is released from the liquid reservoir and migrates over the buffer pad, the liquid may hydrate the buffer pad, dissolving the buffer components and forming a buffer. In some embodiments, the liquid dissolves or suspends a non-target analyte (e.g., volumetric analyte) of known concentration in the liquid. In alternative examples, the liquid contains a non-target analyte (e.g., volumetric analyte) of known concentration prior to the liquid's release from the liquid reservoir. In still further examples, no non-target analyte (e.g., volumetric analyte) is introduced to the liquid.

In the example shown in FIGS. 7 and 8, the analysis device 220 has a front end 250, a back end 252, a dual conjugate pad comprising a first conjugate pad 232 positioned at the first end 234 of the test sample element 230 and a second conjugate pad 236 positioned at the second end 239 of the test sample element. As the buffer moves across the first conjugate pad 232, a first conjugate 238 (e.g., a target analyte conjugate) elutes and moves onto the test sample element where the conjugate binds to the target analyte (if present) in the sample. In some embodiments, a second conjugate (e.g., a non-target analyte conjugate) may bind a non-target analyte (e.g., volumetric analyte). The buffer further migrates across the test sample element 230, eluting the target analyte (and, in some embodiments, the non-target/volumetric analyte) with it. If a portion of the non-target analyte (or volumetric analyte) does not bind to the conjugates (e.g., the second conjugate) in the first conjugate pad 232, it may bind to the conjugates (e.g., the second conjugate) in the second conjugate pad 236 as the buffer elutes the target analyte (and, in some embodiments, the non-target/volumetric analyte) across the second conjugate pad 236 toward the test strip 240. The test strip includes a plurality of bands, e.g. a control band 244, a test band 246, and/or in some embodiments, a non-target analyte band (e.g., volumetric band) (not shown). In embodiments in which a wicking element 242 is present, the wicking element can expedite the flow of the buffer and eluted target analyte (and, in some embodiments, the non-target (e.g., volumetric) analyte). The wicking element may also prevent backflow toward the liquid reservoir 222.

As shown illustratively in FIG. 8, although not necessarily drawn to scale, in some cases the back edge (i.e., closer to back end 250, or downstream edge) of certain components of the analysis device 220 may slightly overlap the front edge (i.e. closer to front end 252, or upstream edge) of the next adjacent component within the device body. This configuration may allow fluid communication between the components of the device.

FIG. 9 is a top schematic view of a further embodiment of an analysis device (e.g., a bodily fluid analysis device) 260. FIG. 10 is a side schematic cross-sectional view of the analysis device 260 of FIG. 9. FIGS. 11A-11E show chronologically successive schematic top views of the analysis device 260 of FIG. 9 in use.

As shown illustratively in FIGS. 9-11E, the bodily fluid analysis device has a front end 290, a back end 292, and a device body 261. As shown illustratively in the figures, the device body may be constructed to analyze multiple target analytes. In this embodiment, the analysis device comprises a plurality of test lines (A, B, C) bordered by control lanes (Q1, Q2), although different configurations of lines/lanes may be present in other embodiments. Each test line is configured for a particular target analyte (e.g., target analyte A, target analyte B, target analyte C). The antibody (and/or antigen) in each test line is chosen to selectively react with or bind the target analyte (target analyte A, target analyte B, target analyte C).

Additionally, in this particular embodiment, the analysis device includes a two-part liquid reservoir including a liquid reservoir 262 that holds a liquid 264 (e.g., water or other suitable liquid) and a buffer pad 266 with buffer components 268 (e.g., one or more salts, sugars, proteins, surfactants, polymers, etc.) deposited onto it. The liquid reservoir may have a breakable seal (e.g., as on a blister pack) as described herein. The buffer pad may be constructed of any suitable material as described herein. When the liquid is released from the liquid reservoir and migrates over the buffer pad, the liquid may hydrate the buffer pad, dissolving or suspending the buffer components and forming a buffered solution 270.

As the buffered solution elutes the analytes from the test sample element 272 and into the lateral flow assay 274, each test band (274A, 274B, 274C) selectively determines or detects the corresponding target analyte (target analyte A, target analyte B, target analyte C). An optional wicking element 275 may be used to expedite flow by drawing the buffered solution and eluted target analytes across the analysis device, and preventing backflow.

As shown in FIGS. 11A-11E (and with reference to FIGS. 9 and 10), the liquid 264 flows from the liquid reservoir 262 into the buffer pad 266 where the liquid dissolves or suspends the buffer components (and/or any other components), forming the buffered solution 270. The buffered solution then migrates into the first conjugate pad 276, eluting the conjugate 278. The buffer 270 and conjugate 278 migrate onto the test sample element 272 that comprises the sample 280 (e.g., sweat sample, or any other suitable bodily fluid sample) and target analyte(s) (if present). The conjugate 278 (alternatively, including conjugates 278A, 278B, 278C, respectively for different target analytes) binds to the target analyte(s) in the sample. Any analyte(s) that remain unbound after passing across the first conjugate pad may bind with conjugate 278 in the second conjugate pad 282 and then flow to the test strip 284. The test band 274 (alternatively, test bands 274A, 274B, 274C) may capture any analyte-conjugate complexes, which may induce an observable change in the test band (e.g., change in color, intensity, fluorescence, etc.). Since target analyte conjugates are not present in control lanes Q1 and Q2, if the lateral flow assay works properly to completion, free conjugates from the first conjugate pad 276 and/or the second conjugate pad 282 may reveal control bands 286 Q1 and 286 Q2 in the Q1 and Q2 regions of the test strip 284, respectively. That is, an observable change in one or more of the control bands can indicate that the assay works properly to completion.

As shown illustratively in FIG. 10, although not necessarily drawn to scale, in some cases the back edge (i.e., closer to back end 292, or downstream edge) of certain components of the analysis device may slightly overlap the front edge (i.e. closer to front end 290, or upstream edge) of the next adjacent component within the device body. This configuration may allow fluid communication between the components of the device.

In some embodiments of an analysis device, such as the devices shown in FIGS. 9-11E, the liquid in the liquid reservoir contains a volumetric analyte, and/or a volumetric analyte is introduced to the liquid following its release from the liquid reservoir. For instance, in certain embodiments the volumetric analyte may be present along with one or more buffer components present in or on a buffer pad or any other suitable component of the device. For example, the volumetric analyte and/or one or more buffer components may be adjacent one or more lines/lanes (e.g., Q1, A, B, C, Q2) shown illustratively in FIG. 9-11E. In some embodiments, the volumetric analyte binds a conjugate specific to the volumetric analyte, the volumetric analyte present (e.g., having been previously deposited) in one more lines/lanes (e.g., Q1, A, B, C, Q2). The one or more volumetric bands may appear on the test strip (e.g., test strip 284) as an observable change as described herein. From this observable change, the volume of the sample (e.g., bodily fluid sample) and/or the volume of the sample in one or more particular lines or lanes (e.g., Q1, A, B, C, Q2) can be determined. For example, each volumetric band may indicate the volume of the sample that was introduced to one of the lines (A, B, C) or lanes (Q1, Q2) in which the volumetric band appears.

It should be understood that while certain features of an analysis device are shown with respect to particular analysis devices shown in the figures, such features can be applied to, or may be present in, other analysis devices described herein. For example, while two separate conjugate regions are described with respect to the analysis device is shown in FIGS. 7-11E, such features may be present in the other analysis devices described herein (e.g., the devices described and/or shown in FIGS. 1A-1C, 4-6, and 12-23). Other configurations are also possible.

In some particular embodiments, one or more of the devices shown in the figures, or described with respect to the figures (e.g., any one of FIGS. 1A-23), may be designed to be worn by the user. For instance, the device may include an adhesive or any other suitable component for attaching the device to the user. In other embodiments, one or more devices are designed to not be worn by the user. In some such embodiments, a sample may be applied directly to the test sample element. In other embodiments, the test sample element may be removably attached to the device. For instance, the test sample element may be used to collect a sample from the user, and may be positioned in a region or reservoir of the device during use. Other configurations are also possible.

FIG. 12 is a top view showing an embodiment of a sweat analysis device 301 of the present disclosure. The sweat analysis device 301 includes a base/device body 310 and various components positioned on the base/device body 310. The components may include a sweat pad 320 (e.g., a test sample element), a conjugate pad 330 (e.g., as part of a conjugate region), a test strip 340, a liquid reservoir 350 (e.g., a buffer reservoir), and an optional wicking element 360. The sweat analysis device may further include a cover layer 370, which may be transparent (FIG. 20). It should be appreciated that while the sweat analysis device is described specifically for obtaining a sweat sample, in other embodiments the device can be used to analyze and/or collect other body fluid samples (e.g., blood, urine, lavage, plasma, saliva, tears).

The sweat analysis device 301 may be assembled by positioning the components in their respective regions relative to each other and optionally covering the components with a cover layer 370. As shown illustratively in FIG. 13, the base/device body 310 has an opening 312, which may be rectangular as shown or may be of any other suitable shape, such as oval or circular. The sweat pad 320 (e.g., a test sample element) may be positioned over the opening 312 so that the sides of the sweat pad 320 overlap with the base/device body, as seen in the bottom (“skin side”) view of FIG. 21. In certain embodiments, such as in some instances in which the device is designed to be worn by the user, one purpose of the opening 312 may be to allow the sweat pad 320 to contact the skin 380 when the sweat analysis device 301 is in use (see FIG. 22).

The base/device body may further include a conjugate region 313 (FIG. 13) where the conjugate pad 330 is positioned, a test region 314 where the test strip 340 is positioned, a liquid reservoir region 315 where the liquid reservoir 350 is positioned, and a wicking region 316 where the wicking element 360 is positioned. The various regions of the sweat analysis device 301 may be sized to fit the corresponding component. For example, the test region 314 may have a length L314 and width W314 that are greater than or equal to the length L340 and width W340 of the test strip 340 (FIG. 17), respectively. Similarly, the length L315 and width W315 of the liquid reservoir region may be greater than or equal to the length L350 and width W350 of the liquid reservoir (FIG. 18A), respectively, and the length L316 and width W316 of the wicking region 316 may be greater than or equal to the length L360 and width W360 of the wicking element (FIG. 19), respectively. In the exemplary embodiment shown, the shape and size of the cover layer 370 (FIG. 20) corresponds to the shape and size of the base/device body so that one or more of the base/device body and its components are covered by the cover layer 370. According to at least one embodiment, the cover layer 370 is shaped and sized to cover only some (but not all) of the components, such as the sweat pad 320 (e.g., a test sample element). In other embodiments, the cover may be adapted and arranged to cover all components of the device.

The components of the sweat analysis device 301 are positioned on the base/device body 310 such that the liquid reservoir 350 is in fluid communication/fluid contact with (e.g., in adjacent contact with or overlaps with) the first end 321 of the sweat pad 320 (e.g., a test sample element). The second end 322 of the sweat pad 320 may be in fluid communication/fluid contact with the first end 331 of the conjugate pad 330, the second end 332 of the conjugate pad 330 may be in fluid communication/fluid contact with the first end 341 of the test strip 340, and the second end 342 of the test strip 340 may be in fluid communication/fluid contact with the wicking element 360.

The base/device body 310 may comprise any suitable material as described herein. In some cases material may be a polymeric material or a combination of polymeric materials, such as polyester, polystyrene, polyvinyl chloride (PVC), polyethylene, polypropylene, and combinations thereof. The thickness of the base/device body (e.g., polymeric material) may vary, as described herein. In some particular embodiments, the thickness may be about 0.05-1.5 mm, about 0.075-1.0 mm, or about 0.1-0.5 mm. Selection of the thickness may depend on the polymeric material selected. For example, a base/device body made of polyester may be thinner (e.g., 0.125-0.25 mm) than a base/device body made of polystyrene or PVC (e.g., 0.25-0.5 mm), although other configurations are also possible. In some cases, the base/device body may have a multi-layered structure, in which one or more layers provides the base/device body with structural integrity, and/or one or more layers provides the base/device body with a particular surface charge. In at least one embodiment, the base/device body comprises a material that is non-permeable to water and aqueous liquids.

In some embodiments, (e.g., in embodiments in which the device is adapted and arranged to be worn by a user), the base/device body may further include a top adhesive layer 411 deposited on the top surface 311 and a bottom adhesive layer 481 deposited on the bottom surface 318. The bottom adhesive layer 481 may adhere the sweat analysis device to the skin 380 during sweat collection (FIG. 22). According to at least one embodiment, other components of the sweat analysis device may be positioned on the top surface 311 of the base/device body 310, and/or are adhered to the base/device body 310 by the top adhesive layer 411. The top adhesive layer 411 may comprise any suitable adhesive that does not interfere with the analysis. For example, the top adhesive layer 411 may comprise a medium-tack, pressure-sensitive adhesive, such as GL187 available from G&L Adhesives Research, Glen Rock, Pa. The sweat analysis device 301 may be provided with a protective layer 371 that covers the bottom adhesive layer 481. The protective layer 371 may be peeled off prior to application of the sweat analysis device 301 to the skin 380 of a subject.

FIGS. 15A and 15B show an exemplary embodiment of the sweat pad 320 (e.g., a test sample element). The sweat pad 320 may be any suitable shape, for example a rectangle or a modified rectangle with tapered corners, as shown. In at least one embodiment, the sweat pad 320 includes an absorbent layer 323 having an absorbent, wicking, and/or capillary property. In some cases, the sweat pad 320 comprises a low-binding, hydrophilic material that is pliable and capable of forming to the contours of the skin 380 (e.g., on an arm) of the subject. Examples of such materials are woven or nonwoven materials of natural or synthetic fibers, such as rayon, polyester, polyurethane, polyolefin, cellulose, cellulose derivatives, cotton, orlon, nylon, hydrogel polymeric material, and combinations thereof, as well as other materials described herein.

In some embodiments, the sweat pad 320 may further comprise a contact membrane 324 that separates the absorbent layer 323 from the skin 380 of the subject. In at least one embodiment, the contact membrane 324 may reduce skin irritation and/or prevent loss of sample or analyte from the sweat pad 320 back onto the skin. The contact membrane 324 may be permeable and may optionally be permeable in one direction only, allowing absorption of sweat into the sweat pad 320 but preventing flow from the sweat pad 320 back onto the skin 380. In an exemplary embodiment, the contact membrane 324 comprises a discontinuous (e.g., porous) polyolefin layer. In other embodiments, the sweat pad does not include a contact membrane.

FIG. 16 shows an exemplary embodiment of a conjugate pad 330. The conjugate pad 330 may be any suitable shape, for example a modified triangular shape as shown in the figures. The conjugate pad 330 comprises a conjugate 333 (e.g., a target analyte conjugate and/or a non-target analyte conjugate) selected at least in part based on the target analyte and/or the analysis method used (e.g., an immunological method or a chemical assay). For example, the conjugate 333 may comprise an antibody bound to a label (i.e., labeled). The conjugate 333 may be labeled with any suitable type of label, such as an enzyme, a fluorescent label (e.g., a fluorescent nanoparticle), a gold particle, or other entity as described herein. In at least one embodiment, the conjugate 333 comprises nanoparticles, (e.g., colloidal gold nanoparticles that are bound to an antibody).

To prepare the conjugate pad 330, the conjugate 333 may be deposited on a pre-treated base layer made of any suitable material, such as glass fiber or a polymeric material (e.g., polyester), or rayon. Other materials can also be used as described herein. The base layer can be pre-treated before application of the conjugate 333 by applying a solution onto the base layer and drying. The solution may comprise, for example, an aqueous solution of one or more salts, sugars, proteins, surfactants, and polymers. In an exemplary embodiment, the solution comprises about 10-200 mM phosphate, 0.2-3% bovine serum albumin (BSA), about 0.2-3% Tween 20, and about 0.1-2% polyvinyl alcohol (PVA). For example, the solution may comprise about 50 mM phosphate, about 1% BSA, about 1% Tween 20, and about 0.5% PVA. Such a method for preparing the conjugate pad may be applied to conjugate pads of other analysis devices described herein.

In an alternative embodiment, the sweat analysis device 301 is configured as a chemical assay, wherein the conjugate pad 330 and conjugate 333 are replaced by a reagent pad and suitable reagents deposited on the reagent pad, respectively.

FIG. 17 shows an exemplary embodiment of a test strip 340 used in the sweat analysis device 301. Such a test strip may be present in other analysis devices described herein, including those designed to analyze other bodily fluid samples besides sweat. According to at least one embodiment, the test strip 340 comprises an immunological assay that may be selected for a particular target analyte, such as a particular drug or other component. In this exemplary embodiment, the test strip 340 comprises a test band 343 and a control band 344. The test strip 340 may also comprise additional bands, (e.g., additional test bands, non-target analyte bands (e.g., a volumetric band)), or may comprise only a test band 343. In some embodiments, the test strip 340 may be configured to provide a positive/negative (true/false) result. In other embodiments, the test may be configured to provide a quantitative result, such as a measure of quantity and/or concentration of a target analyte.

The test strip 340 may comprise any suitable material, as described herein, and in some cases may be porous, having a suitable average pore size as described herein.

FIGS. 18A and 18B show a top view and a cross sectional view, respectively, of an exemplary embodiment of a liquid reservoir 350. Such a liquid reservoir may be present in any analysis device described herein in certain embodiments, including those designed to analyze other bodily fluid samples besides sweat. The liquid reservoir 350 may comprise, for example, a cover layer 352 and a bottom 353 with an inside volume of V350, wherein the liquid reservoir is capable of holding a liquid 351 (e.g., buffer) and then releasing the liquid (e.g., buffer) when the liquid reservoir is broken, pierced, or punctured. In some cases, the liquid reservoir is in the form of a blister pack, though other configurations are also possible as described herein. The cover layer 352 and optionally the bottom 353 may be constructed of a pliable plastic, such as polyethylene or polypropylene, or a foil. The liquid 351 (e.g., buffer) may be any suitable liquid or buffer selected for the target analyte, as described herein.

FIG. 19 shows an exemplary embodiment of the wicking element 360. The wicking element may be of any suitable shape, such as the rectangular shape shown. The wicking element 360 may comprise a wicking or absorbent material that draws in the target analyte and the liquid/buffer (e.g., from an upstream position to a downstream position of the device). Suitable wicking materials may include, for example, cellulose (e.g., high density cellulose), cotton, and other natural and artificial absorbent materials. The wicking element may have any suitable thickness, such as about 0.2-3 mm or about 0.4-2 mm.

In at least one embodiment, the sweat analysis device 301 includes a barrier (not shown) between the sweat pad 320 (e.g., a test sample element) and the conjugate pad 330 that minimizes unintended flow before the buffer is released—e.g., when an excessive amount of sweat is absorbed into the sweat pad 320. The barrier may comprise, for example, glass fiber, polyester, or rayon. In some cases, the barrier may be a semi-permeable membrane. Such a barrier may be present in any analysis device described herein in certain embodiments, including those designed to analyze other bodily fluid samples besides sweat.

FIG. 20 shows an exemplary embodiment of the cover layer 370. Such a cover layer may optionally be present in any analysis device described herein, including those designed to analyze other bodily fluid samples besides sweat. The cover layer 370 may be transparent, or may comprise transparent and non-transparent areas, or may be non-transparent. In at least one embodiment, the cover layer 370 covers one or more of the components of the sweat analysis device 301. For example, the cover layer 370 can be constructed to cover the sweat pad 320 (e.g., a test sample element), the conjugate pad 330, the test strip 340, the wicking element 360, and combinations of some or all of such components. The cover layer may be constructed of any suitable material, such as a polymeric material. An example of a suitable material for the cover layer 370 is a non-woven metallocene-polyethylene, although other materials can be used. The cover layer may protect at least the test strip and does not interfere with the analysis.

FIG. 21 illustratively shows a bottom or “skin side” view of the sweat analysis device 301. From the bottom, only the base/device body 310 and a center portion of the sweat pad 320 (e.g., a test sample element) are visible. According to at least one embodiment, the bottom side 318 of the base/device body 310 comprises a bottom adhesive layer 481. The bottom adhesive layer 481 is compatible with application of the device to the skin 380 (FIG. 22). The bottom adhesive layer 481 can adhere to the skin, but can be peeled away from the skin when the sweat analysis device is removed from the subject. Depending on the intended use of the sweat analysis device, the bottom adhesive layer may comprise an adhesive that adheres to the skin only once, (i.e., the device cannot be removed and re-attached). Suitable adhesive materials include, for example, acrylic pressure-sensitive adhesives, elastomers, and combinations thereof. The bottom adhesive layer may also be covered by a protective cover layer prior to use.

FIG. 22 shows an exemplary embodiment of the sweat analysis device 301 in use on a subject. To obtain a sample of sweat from the subject, the sweat analysis device 301 can be attached to the skin by the bottom adhesive layer 481. Attachment can be achieved at any suitable location on the subject, such as an arm (e.g., upper arm, lower arm, or wrist), hand, back, chest, forehead, leg, foot, etc. In some embodiments, the chosen attachment location is convenient and provides adequate sweat production. For example, in one embodiment, the sweat analysis device 301 is attached to the lower side of the wrist of a subject. According to at least one embodiment, a sweat sample 400 is obtained by absorption, wicking, or capillary action so that sweat from the surface of the skin 380 is absorbed into the sweat pad 320. The sweat analysis device 301 may remain on the skin for as long as needed to collect an adequate sample. For example, the sweat analysis device 301 may remain on the skin for at least 1 minute, at least 3 minutes, at least 5 minutes, at least 10 minutes, at least 15 minutes, at least 30 minutes, at least 60 minutes, at least 1 day, at least 1 week, or at least 1 month. In at least one embodiment, the sweat analysis device 301 remains on the skin for up to about 2 hours, up to about 6 hours, up to about 12 hours, up to about 18 hours, up to about 24 hours, up to 1 week, or up to 1 month. The duration may depend on the subject and the intended use (e.g., immediate testing, monitoring over time, etc.).

After the sweat sample 400 has been collected, the analysis procedure can be started. For example, the analysis procedure can be started immediately or after a period of delay, depending on the intended use, the target analyte, and/or the chosen analysis method. In at least one embodiment, the analysis procedure starts within about 5 minutes, about 10 minutes, about 30 minutes, about 1 hour, about 4 hours, about 24 hours, about 1 week, or about 2 weeks after collecting the sample. To help prevent loss of the sweat sample 400 or the analyte after sample collection and prior to analysis, the sweat analysis device 301 may be temporarily placed on a surface (e.g., a level surface). In some embodiments, the surface is non-permeable and non-absorbent. In at least one embodiment, the sweat analysis device 301 is placed on a level surface regardless of the permeability, absorbance, or cleanliness of the surface. In an exemplary embodiment, a protective layer covers a side (e.g., the bottom side, or side exposing the sweat pad/test sample element) of the sweat analysis device during this period prior to analysis.

FIG. 23 shows a schematic diagram of an exemplary embodiment of an analysis procedure. Once the sweat sample 400 has been obtained, and in some cases after the sweat analysis device 301 has been removed from the skin of the subject, the buffer 351 is released from the liquid reservoir 350. In the exemplary embodiment shown, the liquid reservoir 350 comprises a blister pack, and the buffer 351 is released by breaking the blister (e.g., a portion of a cover layer). Because the liquid reservoir 350 is in adjacent contact with the sweat pad 320 (e.g., a test sample element), the buffer 351 will flow toward and into the sweat pad 320 and in the direction of the second end 322 of the sweat pad (e.g., a downstream end). The wicking action of the wicking element 360 may further draw the buffer 351 toward the wicking element 360.

As the buffer 351 flows across the sweat pad 320, the buffer 351 elutes the sweat sample 400 (including the target analyte and any non-target analytes), causing the sweat sample 400 to also flow toward the wicking element 360. After the sweat sample 400 reaches the second end 322 of the sweat pad 320, it flows onto the conjugate pad 330, where the analyte (if present) binds to the conjugate agent 333, forming an analyte-conjugate complex. If any non-target analytes as well as their conjugates are present, formation of non-target analyte-conjugate complexes may take place. The sweat sample 400, along with the analyte-conjugate complex (and/or any non-target analyte-conjugate complexes), further flows onto and across the test strip 340 where the analyte-conjugate complex reacts with the test band 343. According to at least one embodiment, the reaction or binding of the analyte-conjugate complex with the test band 343 renders the test band 343 visually detectible, indicating that the sweat sample 400 contains a threshold concentration of the target analyte. The presence of free conjugate from the conjugate pad 330 may activate the control band 344, indicating proper functioning of the test. If any non-target analyte-conjugate complexes are present, they may be captured at a non-target analyte band (e.g., a volumetric band).

In some embodiments, an analysis device described herein may be a part of a kit used for determining one or more target analytes. The kit may include, for example, a device comprising a liquid reservoir containing a liquid, wherein the liquid reservoir is sealed prior to use and is adapted and arranged to release the liquid during use. The device may include a region adapted and arranged for housing a test sample element, wherein the region is positioned downstream of the liquid reservoir. The device may include a conjugate region comprising at least a first conjugate adapted and arranged to bind to a target analyte (e.g., a first target analyte), wherein the conjugate region is positioned downstream of the liquid reservoir. Optionally, the device may include a second conjugate adapted and arranged to bind to a second target analyte. Additionally or alternatively, the device may include a conjugate adapted and arranged to bind to a non-target analyte as described herein. The device may include a lateral flow assay, the lateral flow assay comprising a test strip comprising a first band adapted and arranged to capture the target analyte (and optionally, other bands for capturing a second target analyte, a non-target analyte, and/or a control band).

The kit may also include a test sample element. In some embodiments, the kit is packaged such that the test sample element is separate from the device (e.g., the test sample element is not in fluid communication with another component of the device). For instance, the test sample element may be independently packaged and sealed. Upon use, a user may unseal the package and use the test sample element to collect a sample. The test sample element containing the sample can then be introduced into the device as described herein. As described herein, in some cases the test sample element is an absorbent material for absorbing a fluid sample.

In other embodiments, the test sample element may be connected to the device such that the device and test sample element are packaged and/or sealed together. In some such embodiments, the test sample element may be in fluid communication with another component of the device while packaged (e.g., an absorption element, a conjugate region). In some cases (e.g., in certain embodiments in which the device is configured to be worn by a user), a film or cover covering all or portions of the device (e.g., the test sample element) may be removed by the user upon use. Removal of the film or cover may, for example, expose the test sample element so that it can be used to collect a sample as described herein, and/or expose an adhesive so that the device can be applied to the skin of a user. Other configurations are also possible.

Any suitable materials may be used to package and/or seal the device or components of a device. In some embodiments, a package, seal or cover is chosen such that it is substantially impermeable to water vapor. For instance, a material known to provide a high vapor barrier, such as metal foil, certain polymers, certain ceramics and combinations thereof may be used.

In some embodiments, a device described herein (e.g., an analysis device and/or detector/reader) is a part of a system for determining one or more analytes in a sample (e.g., bodily fluid sample). The system may optionally include a control system. The control system may be operatively associated with a one or more components of the device(s) according to one set of embodiments. Control systems described herein can be implemented in numerous ways, such as with dedicated hardware or firmware, using a processor that is programmed using microcode or software to perform the functions recited above or any suitable combination of the foregoing. A control system may control one or more operations of a single analysis, or of multiple (separate or interconnected) analyses. In one example, the control system may be configured to communicate with one or more of the identification component of the analysis device, the detector/reader, and/or other components.

In one embodiment, the control system includes a processor, such as a real time processor that controls and/or monitors one or more components and/or devices (e.g., a detector). If more than one processor is present, communication between these processors may occur, for example, through a serial communication bus.

In some embodiments, the analysis device and/or detector is capable of interfacing with external devices and may, for example, include ports for connection with one or more external communication units. External communication may be accomplished, for example, via USB communication. Additionally, the data stream (e.g., produced by a real time processor) may be outputted to a computer or any other suitable device. Further, other types of communication options are available as the present invention is not limited in this respect. For example, Ethernet, Bluetooth and/or WI-FI communication may be established through the processor.

The determination methods, steps, calculation methods, systems, and operation of components described herein may be implemented using a computer implemented control system, such as the various embodiments of computer implemented systems described below. The methods, steps, systems, and systems described herein are not limited in their implementation to any specific computer system described herein, as many other different machines may be used.

The computer implemented control system can be part of or coupled in operative association with an analysis device and/or detector, and, in some embodiments, configured and/or programmed to control and adjust operational parameters of the analysis device and/or detector, as well as analyze and calculate values, as described herein. In some embodiments, the computer implemented control system can send and receive reference signals to set and/or control operating parameters of the analysis device and/or detector and, optionally, other system apparatus. In other embodiments, the computer implemented system can be separate from and/or remotely located with respect to the analysis device and/or detector and may be configured to receive data from one or more remote analysis devices and/or detectors via indirect and/or portable means, such as via portable electronic data storage devices, such as magnetic disks, or via communication over a computer network, such as the Internet or a local intranet.

The computer implemented control system may include several known components and circuitry, including a processing unit (i.e., processor), a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as described herein. Further, the computer system may be a multi-processor computer system or may include multiple computers connected over a computer network.

The computer implemented control system may include a processor, for example, a commercially available processor such as one of the series x86, Celeron and Pentium processors, available from Intel, similar devices from AMD and Cyrix, the 680X0 series microprocessors available from Motorola, and the PowerPC microprocessor from IBM. Many other processors are available, and the computer system is not limited to a particular processor.

A processor typically executes a program called an operating system, of which WindowsNT, Windows95 or 98, UNIX, Linux, DOS, VMS, MacOS and OS8 are examples, which controls the execution of other computer programs and provides scheduling, debugging, input/output control, accounting, compilation, storage assignment, data management and memory management, communication control and related services. The processor and operating system together define a computer platform for which application programs in high-level programming languages are written. The computer implemented control system is not limited to a particular computer platform.

The computer implemented control system may include a memory system, which typically includes a computer readable and writeable non-volatile recording medium, of which a magnetic disk, optical disk, flash memory, and tape are examples. Such a recording medium may be removable, for example, a floppy disk, read/write CD or memory stick, or may be permanent, for example, a hard drive.

Such a recording medium stores signals, typically in binary form (i.e., a form interpreted as a sequence of ones and zeros). A disk (e.g., magnetic or optical) has a number of tracks, on which such signals may be stored, typically in binary form, (i.e., a form interpreted as a sequence of ones and zeros). Such signals may define a software program, (e.g., an application program), to be executed by the microprocessor, or information to be processed by the application program.

The memory system of the computer implemented control system also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer implemented control system that implements the methods, steps, systems and devices described herein is not limited thereto. The computer implemented control system is not limited to a particular memory system.

At least part of such a memory system described above may be used to store one or more data structures (e.g., look-up tables) or equations described above. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer implemented control system may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on a hard disk for use at a later time.

The computer implemented control system may include one or more output devices. Example output devices include a cathode ray tube (CRT) display, liquid crystal displays (LCD) and other video output devices, printers, communication devices such as a modem or network interface, storage devices such as a disk or tape, and audio output devices such as a speaker.

The computer implemented control system also may include one or more input devices. Example input devices include a keyboard, keypad, track ball, mouse, pen and tablet, communication devices such as those described above, and data input devices such as audio and video capture devices and sensors. The computer implemented control system is not limited to the particular input or output devices described herein.

It should be appreciated that one or more of any type of computer implemented control system may be used to implement various embodiments described herein. Aspects of the invention may be implemented in software, hardware or firmware, or any combination thereof. The computer implemented control system may include specially programmed, special purpose hardware, for example, an application-specific integrated circuit (ASIC). Such special-purpose hardware may be configured to implement one or more of the methods, steps, systems, and system elements/components described above as part of the computer implemented control system described above or as an independent component.

The computer implemented control system and components thereof may be programmable using any of a variety of one or more suitable computer programming languages. Such languages may include procedural programming languages, for example, C, Pascal, Fortran and BASIC, object-oriented languages, for example, C++, Java and Eiffel and other languages, such as a scripting language or even assembly language.

The methods, steps, devices, systems, and system elements/components may be implemented using any of a variety of suitable programming languages, including procedural programming languages, object-oriented programming languages, other languages and combinations thereof, which may be executed by such a computer system. Such methods, steps, systems, and system elements/components can be implemented as separate modules of a computer program, or can be implemented individually as separate computer programs. Such modules and programs can be executed on separate computers.

Such methods, steps, systems, and system elements/components, either individually or in combination, may be implemented as a computer program product tangibly embodied as computer-readable signals on a computer-readable medium, for example, a non-volatile recording medium, an integrated circuit memory element, or a combination thereof. For each such method, step, simulation, algorithm, system, or system element, such a computer program product may comprise computer-readable signals tangibly embodied on the computer-readable medium that define instructions, for example, as part of one or more programs, that, as a result of being executed by a computer, instruct the computer to perform the method, step, system, or system element/component.

The following examples are intended to illustrate certain embodiments of the present invention, but do not exemplify the full scope of the invention.

### EXAMPLES

The devices and methods of the present disclosure are next described by means of the following examples. The use of these and other examples anywhere in the specification is illustrative only, and in no way limits the scope and meaning of the invention or of any exemplified form. Likewise, the invention is not limited to any particular preferred embodiments described herein.

### Example 1

### Prophetic

An analysis device (e.g., a bodily fluid analysis device) 11 as shown in FIG. 1C for detecting benzoylecgonine (the primary metabolic product of cocaine) in sweat is configured as follows:

| | |
|---|---|
| Device body (11): | 10 mm thick, made of PVC |
| Test sample element | Absorbent, non-woven, cellulose-based sweat collection pad with a |
| (20): | protective polyolefin layer on the skin side |
| Buffer (16): | 50 mM PBS, 1% BSA, 1% Tween 20, and 0.5% PVA; buffer pH |
| | adjusted to 7.2 and contains a defined concentration of added |
| | volumetric protein analyte |
| Conjugate region (22): | Conjugate pad made of glass fiber, deposited with colloidal gold bound |
| | to an antibody specific to benzoylecgonine and a conjugate capable of |
| | binding the volumetric protein. |
| Test strip (23): | Nitrocellulose strip including a test band (36), volumetric band (40) and |
| | control band (38) |
| Wicking element (35): | High-density cellulose pad |

### Example 2

### Prophetic

An analysis device (e.g., a bodily fluid analysis device) 11 as shown in FIG. 1C for detecting to morphine in sweat is configured as follows:

| | |
|---|---|
| Device body (11): | 10 mm thick, made of PVC |
| Test sample element | Synthetic non-woven polyester absorbent sweat collection pad with a |
| (20): | protective polyolefin layer on the skin side |
| Buffer (16): | 100 mM PBS, 1.5% BSA, 1.5% Tween 20, and 0.7% PVA; buffer pH |
| | adjusted to 7.5 and contains a defined concentration of added |
| | volumetric protein analyte |
| Conjugate region (22): | Conjugate pad made of glass fiber, deposited with colloidal gold bound |
| | to an antibody specific to morphine and a conjugate capable of binding |
| | the volumetric protein analyte |
| Test strip (23): | Nitrocellulose strip including a test band (36), volumetric band (40) and |
| | control band (38) |
| Wicking element (35): | High-density cellulose pad |

### Example 3

### Prophetic

An analysis device (e.g., a bodily fluid analysis device) 11 as shown in FIG. 1C for detecting THC (the active ingredient in cannabis) in sweat is configured as follows:

| | |
|---|---|
| Device body (11): | 10 mm thick, made of PVC |
| Test sample element | Absorbent, non-woven, cellulose-based sweat collection pad with a |
| (20): | protective polyolefin layer on the skin side treated to have neutrally |
| | charged surface |
| Buffer (16): | 50 mM TRIS, 3M urea and 0.1% tween 20; buffer pH adjusted to 7.8 |
| | and contains a defined concentration of added volumetric protein |
| | analyte |
| Conjugate region (22): | Conjugate pad made of FUSION 5 ™, deposited with 2.3 μm beads of |
| | colloidal gold embedded into the FUSION 5 ™ pores and bound to an |
| | antibody specific to THC and a conjugate capable of binding the |
| | volumetric protein analyte |
| Test strip (23): | FUSION 5 ™ strip including a test band (36), volumetric band (40) and |
| | control band (38) |
| Wicking element (35): | High-density cellulose pad |

### Example 4

### Prophetic

An analysis device (e.g., a bodily fluid analysis device) 11 as shown in FIG. 1C for detecting 3,4-methylenedioxymethamphetamine (MDMA or “ecstasy”) in sweat is configured as follows:

| | |
|---|---|
| Device body (11): | 10 mm thick, made of PVC |
| Test sample element | Absorbent, non-woven, cellulose-based sweat collection pad with a |
| (20): | protective polyolefin layer on the skin side |
| Buffer (16): | 75 mM PBS, 1% BSA, 1% Tween 20, and 0.5% PVA; buffer pH |
| | adjusted to 7.4-7.6 and contains a defined concentration of added |
| | volumetric protein analyte |
| Conjugate region (22): | Conjugate pad made of glass fiber, deposited with colloidal gold |
| | bound to an antibody specific to MDMA and a conjugate capable of |
| | binding the volumetric protein analyte |
| Test strip (23): | Nitrocellulose strip including a test band (36), volumetric band (40) |
| | and control band (38) |
| Wicking element (35): | High-density cellulose pad |

### Example 5

### Prophetic

An analysis device (e.g., a bodily fluid analysis device) 220 as shown in FIGS. 7-8 for detecting erythropoietin (EPO) in sweat is configured as follows:

| Device body (221): | 0.4 mm thick PVC membrane with GL187 adhesive on the top side and |
|---|---|
| | acrylic adhesive on the skin side |
| Test sample | Cellulose-based non-woven absorbent sweat pad with a protective |
| element (230): | polyolefin layer on the skin side |
| Buffer components | 50 mM PBS, 1% BSA, 1% Tween 20, and 100 mM EDTA 0.5% PVA; |
| (228): | buffer pH adjusted to 7.6-7.8 and contains a defined concentration of added |
| | volumetric protein analyte |
| Conjugate pads | Conjugate pad made of glass fiber, deposited with colloidal gold bound to |
| (232 and 236) and | an antibody specific to erythropoietin and a conjugate capable of binding |
| conjugates (238): | the volumetric protein analyte |
| Test strip (240): | Nitrocellulose strip with test band, control band, and volumetric band, the |
| | test band will have an antibody specific to EPO |
| Wicking element | High-density cellulose pad |
| (242): | |

### Example 6

### Prophetic

An analysis device (e.g., a bodily fluid analysis device) 260 as shown in FIGS. 9-11 for detecting multiple analytes in sweat is configured as follows:

| Device body (261): | 0.4 mm thick PVC membrane with GL187 adhesive on the top side and |
|---|---|
| | acrylic adhesive on the skin side |
| Test sample | Cellulose absorbent sweat pad with a protective polyolefin layer on the |
| element (272): | skin side treated to have neutrally charged surface |
| Liquid 264 and | Water containing 100 mM phosphate, 1% BSA, 1% Tween 20, and 0.5% |
| buffer components | PVA; buffer pH adjusted to 7.4-7.6. 5 and contains a defined concentration |
| (268): | of added volumetric protein analyte |
| Conjugate pads | Conjugate pad made of glass fiber, deposited with colloidal gold bound to |
| (276) and | antibodies specific to the target analytes, each conjugate deposited in the |
| conjugates (278): | specific lane for the corresponding target analyte and a conjugate capable |
| | of binding the volumetric protein analyte |
| Test strip (284): | Nitrocellulose strip with multiple test bands, at least one control band, and |
| | at least one volumetric band, the test band having lane-specific antibodies |
| Wicking element | High-density cellulose pad |
| (275): | |

### Example 7

### Prophetic

An analysis device (e.g., a bodily fluid analysis device) 11 as shown in FIG. 1C for detecting ethyl glucuronide (a metabolite of ethyl alcohol) in sweat is configured as follows:

| | |
|---|---|
| Device body (11): | 10 mm thick, made of PVC |
| Test sample element | Cellulose-based non-woven sweat absorbent pad with a protective |
| (20): | polyolefin layer on the skin side |
| Buffer (16): | 50 mM PBS, 1% BSA, 1% Tween 20, and 0.5% PVA; buffer pH |
| | adjusted to 7.2 and contains a defined concentration of added |
| | volumetric protein analyte |
| Conjugate region (22): | Conjugate pad made of glass fiber, blocked with 50 mM PBS, 1% |
| | BSA, 1% Tween 20, and 0.5% PVA; buffer pH adjusted to 7.2; |
| | deposited with colloidal gold bound to an antibody specific to |
| | ethylglucuronide and a conjugate capable of binding the volumetric |
| | protein analyte |
| Test strip (23): | Nitrocellulose strip including a test band (36), volumetric band (40) and |
| | control band (38) |
| Wicking element (35): | High-density cellulose pad |

### Example 8

The concentration of delta-9-tetrahydrocannabinot (THC) in sweat was compared to cannabis-induced psychomotor impairment by neuroimaging and evaluating performance of psychomotor tasks relevant to driving a car. The test protocol was designed to empirically test changes in brain activity during cannabis intake compared with baseline (no intake), and to identify correlation with psychomotor performance. The correlation of THC and metabolites concentrations between different biological matrices was studied with special reference to sweat and THC-induced psychomotor impairment. The protocol was as follows:

Subjects: recreational cannabis users of both genders older than 18 years; n=20 subjects

Treatment: subjects are administered a cannabis preparation

Procedure: Subjects were assessed (i) in the absence of drug intake and (ii) after cannabis intake. Half of the study sample started with the absence-of-drug-intake session and half with the cannabis-intake session. Sessions were performed on different days. In each experimental session, fMRI of brain activity were acquired during the execution of selected tasks. Psychomotor measurements of task performance were recorded online during fMRI, and real time psychophysical measurements including heart rate and ventilation (respiratory) oscillations, were recorded as well. Neuroimaging, psychomotor evaluation and biological matrices sampling were performed at time intervals of 0 min, 45 min (peak effects) and 150 min (blood concentrations close to administrative cut-off of THC concentrations of 5 ng/mL).

### Experimental fMRI Protocol

fMRI was acquired using a superconductive magnet 1.5 Tesla Signa Excite system (General Electric, Milwaukee, Wis.). Brain activity was assessed using three different tasks or situations selected on the basis of their potential sensitivity to the effect of cannabis compounds:

1. Resting-state: Continuous acquisition of fMRI in a relaxed rest with closed eyes to evaluate brain activity during cannabis consumption state with respect to baseline using whole-brain measurements of functional connectivity.

2. Critical Tracking Task: an adaptation of the classical Critical Tracking Task (CTT, Systems technology Inc.) was used. In this interactive task, participants were presented with a schematic representation of a running vehicle on the screen and were required to keep the vehicle in the middle of the roadway using adapted hand buttons. The number of errors and the time outside the road were recorded.

3. Movement Estimation Task: this task assessed the subject's ability to estimate the speed of movement, or time to contact of a moving object to a fixed point. A visible schematic vehicle was running at constant speed during a fixed period, after which the vehicle was occluded by a represented tunnel. It traveled underneath and was no longer visible. The subject had to estimate the time the vehicle reached a marked point in the tunnel and indicate the moment by pressing a button. Absolute time to contact error, defined as the absolute mean difference between estimated and actual time, was registered.

Analysis: each fMRI task was analyzed with specific software to generate whole-brain maps of brain activity. Functional brain maps during baseline and cannabis session were statistically compared. In addition, specific maps of the correlation between brain activity and performance measurements were generated.

Biological Matrices: THC concentration was measured was sweat and blood

Collection of Samples and Data:

Baseline data was collected prior to administering cannabis to the subjects. A first sweat pad was placed on a subject's skin and was worn by the subject for fifteen minutes to collect a baseline sample. The first sweat pad including the collected sweat sample was then removed and THC concentration in the collected sweat was analyzed. Blood was also collected for testing. The subject underwent psychomotor testing and was administered a fMRI scan.

At time 0 minutes, cannabis was self-administered to the subject. Thirty minutes after cannabis was administered to the subject, a second sweat pad was placed on the subject's skin and is worn by the subject for 15 minutes. The second sweat pad was removed and THC concentration was analyzed. Blood was also collected and the psychomotor testing and fMRI were repeated.

A third sweat pad was placed on the subject's skin after administering cannabis was is worn by the subject for 15 minutes. The third sweat pad was removed at time 150 min and THC concentration was analyzed. Blood was also collected and the psychomotor testing and fMRI were repeated.

Drug analysis: THC amounts in sweat and blood was determined by HPLC coupled to tandem mass spectrometry (LC/MS/MS).

From the results of this test, it was shown that it is possible to measure THC excreted through sweat in relatively short periods of time (e.g., ˜15 mins) after collecting a sweat sample from a subject. This example also shows that concentrations of THC in sweat correlated with concentrations of THC in plasma. This data suggests it may be possible to determine a certain minimum of THC concentration from a sweat sample (e.g., using an analysis device described herein), that could indicate a particular level of impairment in the subject.

### Example 9

This example shows the use of an analysis device to determine the concentration of a target analyte, benzoylecgonine (a metabolite of cocaine), in a sweat sample. The device had a configuration similar to the device configuration shown in FIG. 1C.

The device included a liquid reservoir (blister pack) containing a phosphate buffered solution having a total volume of 250 μL. The buffer solution contained a known concentration (2.2 mg/mL) of D-biotin, which was used as a volumetric analyte (non-target analyte) in the assay. The device also included a test sample element (absorbent pad), a conjugate region including a first conjugate (a test analyte conjugate, which was anti-benzoylecgonine/cocaine labeled with a colloidal gold) and a second conjugate (a volumetric analyte conjugate, which was anti-D-biotin labeled with a colloidal gold). The device also included a lateral flow assay including a test strip. The test strip included a control band, a test band, and a volume band. A wicking pad was positioned at a downstream end of the device.

The test band was configured to detect benzoylecqonine (a target analyte) using a competitive assay. In the competitive assay, the target analyte in the sample and the molecules immobilized at the test band (cocaine-BSA) compete for the test analyte conjugate (labelled anti-benzoylecgonine/cocaine, initially positioned in the conjugate region). Thus, the greater amount of target analyte in the sample, the greater amount of binding between the target analyte and anti-benzoylecgonine/cocaine test analyte conjugate, leaving less of the test analyte conjugate available for binding/capture at the test band. Accordingly, a lower intensity of the test band indicates a higher amount/concentration of target analyte in the sample (i.e., the intensity of the test band is inversely correlated with the amount or concentration of target analyte in the sample).

The volume band was used as part of a volumetric test to determine the volume of the sample collected using the absorbent pad. In this test, the known volumetric analyte in the buffer solution and the immobilized molecules at the volume band (biotin-BSA) compete for the same volumetric analyte conjugate (initially positioned in the conjugate region). Thus, the greater the sample volume, the more dilute the sample/buffer mixture. There will be relatively less of the volumetric analyte present for binding with its conjugate, thereby decreasing the amount of volumetric analyte-conjugate available for binding/capture at the volume band (compared to if a smaller sample volume was present). Accordingly, a lower intensity of the volume band indicates a higher volume of sample (i.e., the intensity of the volume band is inversely correlated with the sample volume).

To start the test, a sweat sample was collected using an absorbent pad for 1 minute. The pad was then placed in a holding region of the device (e.g., a sampling area). The lid was closed and the buffer solution was released from the liquid reservoir by pressing a button on the lid, which punctured the liquid reservoir. The buffer solution mixed with the fluid sample, and the mixture flowed across the conjugate region to allow binding between the target analyte in the sample and the test analyte conjugate to form target analyte/test analyte conjugate complexes, and binding between the volumetric analyte and the volumetric analyte conjugate to form volumetric analyte/conjugate complexes. The mixture containing the bound complexes and the unbound conjugates then flowed to the test strip. Any unbound test analyte conjugates bound to the immobilized molecules at the test band, and any unbound volume conjugates bound to the immobilized molecules at the volume band.

A reader was used to determine the intensities at the control band, volume band, and test band 10 minutes after the sample/buffer mixture was introduced to the test strip. The intensity of the control band indicated that the assay had worked properly. The intensity of the volume band was compared to a calibration curve correlating intensity and volume. This calibration curve was used to determine the sample volume (microliters). The intensity of the test band was compared to a calibration curve correlating intensity and amount of target analyte. This calibration curve was used to determine the sample amount (nanograms). The concentration of the target analyte in the sample (nanograms/microliter) was calculated by dividing the normalized sample volume by the normalized sample amount. In this particular example, the concentration of benzoylecgonine was determined to be 0.1 ng/μL of sweat sample.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of determining a concentration of a target analyte in a bodily fluid sample, comprising:
introducing the bodily fluid sample comprising the target analyte into a bodily fluid analysis device, wherein the bodily fluid analysis device comprises a lateral flow assay;
passing the bodily fluid sample across a test strip comprising a first band and a second band, wherein the first band is adapted and arranged to determine a quantity of the target analyte in the bodily fluid sample, and wherein the second band is adapted and arranged to determine a volume of the bodily fluid sample; and
determining the concentration of the target analyte in the bodily fluid sample based on the steps of determining the quantity of the target analyte and the volume of the bodily fluid sample.

2. A method of determining a concentration of a target analyte in a bodily fluid sample, comprising:
introducing the bodily fluid sample comprising the target analyte into a bodily fluid analysis device, wherein the bodily fluid analysis device comprises a lateral flow assay;
passing the bodily fluid sample across a test strip comprising a first band and a second band, wherein the first band is adapted and arranged to capture the target analyte in the bodily fluid sample, and wherein the second band is adapted and arranged to capture a non-target analyte; and
determining the concentration of the target analyte in the bodily fluid sample based on the steps of capturing the target analyte and non-target analyte.

3. (canceled)

4. The method as in claim 1, wherein the bodily fluid analysis device comprises a sealed liquid reservoir containing a liquid.

5. (canceled)

6. The method as in claim 4, wherein the sealed liquid reservoir comprises a puncturable seal.

7. (canceled)

8. The method as in claim 4, wherein the bodily fluid analysis device comprises a liquid releasing member adapted and arranged to release a liquid from the liquid reservoir.

9. (canceled)

10. The method as in claim 1, wherein the bodily fluid analysis device comprises a device body comprising a region adapted and arranged for housing a test sample element containing a sample, and wherein the region comprises a lid.

11. (canceled)

12. The method as in claim 10, wherein the lid or the device body comprises a liquid releasing member.

13. The method as in claim 10, wherein the lid comprises a compression element for applying a pressure to the test sample element upon closure of the lid.

14. The method as in claim 10, wherein the lid comprises a locking mechanism that prevents opening of the lid after the lid has been closed.

15-16. (canceled)

17. The method as in claim 1, wherein the bodily fluid analysis device comprises a conjugate region comprising a first conjugate adapted and arranged to bind the target analyte and a second conjugate adapted and arranged to bind a non-target analyte.

18-21. (canceled)

22. The method as in claim 17, wherein the first and/or second conjugate comprises a detectable label.

23-25. (canceled)

26. The method as in claim 1, wherein the bodily fluid analysis device comprises a first band adapted and arranged to determine a quantity of the target analyte in the bodily fluid sample and a second band adapted and arranged to determine a volume of the bodily fluid sample.

27-29. (canceled)

30. The method as in claim 26, wherein the test strip displays the first band and the second band, the first band comprising a complex of the target analyte and the first conjugate, the second band comprising a complex of the non-target analyte and the second conjugate,
wherein an intensity of the first band correlates with a quantity of the target analyte in the bodily fluid sample, and
wherein an intensity of the second band correlates with a volume of the bodily fluid sample.

31-35. (canceled)

36. The method as in claim 1, wherein the bodily fluid device comprises a device identifying component that comprises a script for running a particular analysis; and, wherein the device identifying component comprises an RFID, a barcode, a QR code, a Bluetooth-enabled component, or a Wi-Fi-enabled component.

37-39. (canceled)

40. The method as in claim 1, wherein the bodily fluid analysis device comprises a device body comprising a non-permeable layer defining a top and a bottom and an opening.

41-42. (canceled)

43. The method as in claim 1, wherein introducing the bodily fluid sample comprising the target analyte into the bodily fluid analysis device comprises placing a test sample element comprising the bodily fluid sample into a region adapted and arranged for housing the test sample element and sample.

44-50. (canceled)

51. The method as in claim 26, the method comprising determining the difference between a known amount or concentration of the non-target analyte present in the bodily fluid analysis device prior to use, and the amount or concentration of the non-target analyte present at the second band.

52. The method as in claim 51, the method comprising determining the concentration of the target analyte in the bodily fluid sample based on the steps of capturing the target analyte and non-target analyte.

53-54. (canceled)

55. The method as in claim 1, the method comprising determining an indication of drug impairment in a subject.

56. The method as in claim 55, wherein the indication of drug impairment is due to intake by the subject of a composition comprising one or more of benzoylecgonine, morphine, THC, methylenedioxymethamphetamine, EPO, ethyl glucuronide and/or a derivative thereof.

57. The method as in claim 1, wherein the bodily fluid sample comprises sweat, blood, saliva, or urine.

58-73. (canceled)

74. A bodily fluid analysis device for determining a concentration of a target analyte in a bodily fluid sample, comprising:
a lateral flow assay, the lateral flow assay comprising a test strip, wherein the test strip comprises a first band and a second band, the first band adapted and arranged to determine a quantity of the target analyte in the bodily fluid sample, and the second band adapted and arranged to determine a volume of the bodily fluid sample.

75-133. (canceled)
